(19) United States
(12) Patent Application Publication
(10) Pub. No.: US 2024/0209129 A1
(43) Pub. Date: Jun. 27, 2024

(54) OPHTHALMIC LENS MATERIALS AND DEVICES MADE THEREOF

(71) Applicant: Johnson & Johnson Surgical Vision, Inc., Irvine, CA (US)

(72) Inventors: Azaam Alli, Jacksonville, FL (US); Scott Joslin, Ponte Vedra Beach, FL (US); Michael Lopez, Jacksonville, FL (US)

(21) Appl. No.: 18/526,589

(22) Filed: Dec. 1, 2023

Related U.S. Application Data
(60) Provisional application No. 63/429,512, filed on Dec. 1, 2022.

(51) Int. Cl. C08F 20/36; C08K 5/101

(52) U.S. Cl. CPC C08F 20/36; C08K 5/101; C08F 2438/00

(57) ABSTRACT

Disclosed are compositions which are produced from reactive monomer mixtures and which have both high refractive indexes and high Abbe numbers. These materials are well suited for use as implantable ophthalmic devices and have a refractive index which may be edited through the application of energy. When used for an intraocular lens, the high refractive index allows for a thin lens which compresses easily through a small incision. Some compositions are suitable for use as intraocular lenses, phakic intraocular lenses, contact lenses, orthokeratology lenses, rigid gas permeable lenses, corneal inlays, corneal outlays, or corneal inserts.

### CROSS-REFERENCE TO RELATED APPLICATIONS

This application claims priority to U.S. Provisional Patent Application No. 63/429,512 filed on Dec. 1, 2022, the entire contents of which are hereby incorporated by reference.

### FIELD OF THE INVENTION

This invention is directed to compositions produced from reactive monomer mixtures which when polymerized form polymeric networks having high refractive indexes and high Abbe numbers. These materials, which may have an editable refractive index, are designed for use in ophthalmic devices, such as intraocular implants, intraocular lenses, phakic intraocular lenses, contact lenses, orthokeratology lenses, rigid gas permeable lenses, corneal inlays, corneal outlays, or corneal inserts.

### BACKGROUND OF THE INVENTION

Cataract surgery is commonly performed to replace the natural eye lens that has become opaque. Materials that are used to replace the natural crystalline lens must be soft and have excellent flexibility so that, once formed into a lens, they may be folded and passed through an incision which is typically about 2 millimeters. Furthermore, the material must have excellent transparency and little to no glistening. Having a high refractive index allows for a thinner lens to be used. A material with a high Abbe number exhibits less dispersion which in turn allows for improved optical results and less light scattering. Combining a high refractive index with a high Abbe number provides preferable optical characteristics for an ophthalmic material.

One of the first patents in this area, U.S. Pat. No. 4,573,998, to Mazzocco, discloses a deformable intraocular lens that can be rolled to fit through a relatively small incision. The deformable lens is inserted into the eye while it is held in its rolled configuration, then released inside the chamber of the eye. The elastic properties of the lens cause it to resume its molded shape after insertion into the eye. Mazzocco discloses polyurethane elastomers, silicone elastomers, hydrogel polymer compounds, organic or synthetic gel compounds and combinations thereof as suitable materials for the deformable lens.

Intraocular lenses can be damaged during implantation, for instance, by the frictional forces from the delivery device. To overcome this issue, some delivery devices are coated to provide extra lubricity. For example, U.S. Pat. No. 8,323,799, to Hu, discloses a soft, flexible highly lubricious coatings for polymeric insertion cartridges that allow intraocular lenses to be easily inserted through small bore cartridges suitable for use with small (less than 3 mm) incisions. While such coatings are helpful, there is a need for a material that exhibits a balance of physical and mechanical properties not only to enable insertion through a small diameter incision but also to recover its original shape and functionality after placement in the eye.

Accordingly, there is a need for a material with relatively high refractive index and Abbe number which can be formed into a flexible intraocular lens, which can be folded into a compact configuration for insertion through a small incision and then unfolded into its original form at the site of implantation.

### SUMMARY OF THE INVENTION

This invention relates to compositions that are suitable for use in ophthalmic devices, such as intraocular lenses, phakic intraocular lenses, intraocular implants, orthokeratology lenses, contact lenses, corneal inlays, corneal outlays, or corneal inserts, made by free radical polymerization of a reactive monomer mixture comprising:
a) a compatibilizing monomer selected from the group consisting of a pendant carbamate monomer, a pendant amide monomer, and combinations thereof;
b) a cross-linking agent; and
c) an ethylene glycol dicyclopentenyl ether (meth)acrylate;
   wherein the concentration of the ethylene glycol dicyclopentenyl ether (meth)acrylate in the reactive monomer mixture excluding any diluent is greater than or equal to 20 weight percent; and wherein the composition exhibits a refractive index of at least 1.45 and an Abbe number of at least 39.

In one aspect, the pendant carbamate monomer has a chemical structure described by Formula I, P_{g}-L-OCONR¹R². In another aspect, the pendant amide monomer has a chemical structure described by Formula II, P_{g}-L-CONR¹R². In Formula I and Formula II, P_{g} is a polymerizable group, L is a linking group, and R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, and heteroaryl groups.

In some specific aspects of the invention, the compatibilizing monomer has the chemical structures depicted in Formula III and Formula IV

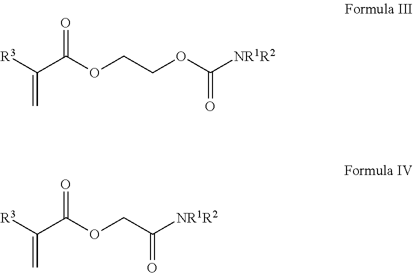

Wherein R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups, and, R³ is H or methyl.

The invention also relates to new compounds that are suitable for the manufacturing of ophthalmic devices by free radical polymerization having the chemical structure shown in Formula IV. For example, homopolymers, copolymers, and crosslinked networks derived from Formula IV monomers can be used to make a variety of ophthalmic devices, such as intraocular lenses, phakic intraocular lenses, intraocular implants, contact lenses, corneal inlays, corneal outlays, or corneal inserts, as well as formulation components such as plasticizing agents in intraocular lenses, wetting agents in contact lenses or eye drops, packaging solution additives for contact lenses, and the like. The copolymers can be block or graft copolymers, including but not limited to diblock and triblock copolymers as well as segmented block copolymers. The crosslinked networks can be water swellable or not water swellable. The crosslinked networks can be hydrogels or silicone hydrogels depending on their composition when the equilibrium water content is sufficiently high. The crosslinked networks can also be used to make orthokeratology lenses and rigid gas permeable lenses. The invention further relates to a method of synthesizing a compound of Formula IV comprising the steps of (a) reacting an amine with methyl glycolate to form a N-alkyl-2-hydroxyacetamide and (b) reacting the N-alkyl-2-hydroxyacetamide with (meth)acryloyl chloride.

In other aspects, the present invention provides a method for making an ophthalmic device, the method comprising the steps of (a) providing a composition comprised of a compatibilizing monomer, a cross-linking agent and an ethylene glycol dicyclopentenyl ether (meth)acrylate and (b) forming an ophthalmic device; alternatively, (a) molding the device from a composition comprised of a compatibilizing monomer, a cross-linking agent and an ethylene glycol dicyclopentenyl ether (meth)acrylate; alternatively, (a) providing a composition comprised of a compatibilizing monomer, a cross-linking agent and an ethylene glycol dicyclopentenyl ether (meth)acrylate in a mold assembly, (b) forming an ophthalmic device, and (c) demolding the ophthalmic device from the mold assembly; and alternatively, (a) providing a composition comprised of a compatibilizing monomer, a cross-linking agent and an ethylene glycol dicyclopentenyl ether (meth)acrylate in a mold assembly, (b) forming an ophthalmic device by a photopolymerization reaction, and (c) demolding the ophthalmic device from the mold assembly.

In certain aspects of any of the above methods of making an ophthalmic device, the method further comprises a step of extracting the ophthalmic device with a solvent; further comprises a step of hydrating the extracted ophthalmic device with at least one aqueous solution; further comprises a step of sterilizing the ophthalmic device; and further comprises an irradiation step using a laser either before or after sterilization, including after the ophthalmic device has been implanted in a human.

### BRIEF DESCRIPTION OF THE FIGURES

FIG. 1 shows the UV-VIS transmission spectra of HEVB & HEVC in 0.2 mM methanol.

FIG. 2 shows the UV-VIS transmission spectra of Examples 8, 10 and 11 Disks.

FIG. 3 shows the UV-VIS transmission spectra of Ex. 12 Disks and Ex. 13 Disks.

FIG. 4 shows the UV-VIS transmission spectra of Examples 126-130 Disks.

### DETAILED DESCRIPTION OF THE INVENTION

### A. Definitions

It is to be understood that the disclosure is not limited to the details of construction or process steps set forth in the following description. The disclosure is capable of other embodiments and of being practiced or being carried out in various ways using the teaching herein.

With respect to the terms used in this disclosure, the following definitions are provided. Unless defined otherwise, all technical and scientific terms used herein have the same meaning as commonly understood by one of ordinary skill in the art to which the disclosure belongs. The polymer definitions are consistent with those disclosed in the Compendium of Polymer Terminology and Nomenclature, IUPAC Recommendations 2008, edited by: Richard G. Jones, Jaroslav Kahovec, Robert Stepto, Edward S. Wilks, Michael Hess, Tatsuki Kitayama, and W. Val Metanomski. All publications, patent applications, patents, and other references mentioned herein are incorporated by reference.

“Target macromolecule” means the macromolecule being synthesized from the reactive monomer mixture comprising monomers, macromers, prepolymers, cross-linkers, initiators, additives, diluents, and the like.

A “macromolecule” is an organic compound having a number average molecular weight of greater than 1500 Daltons and may be reactive or non-reactive. The number average molecular weight, weight average molecular weight, and the polydispersity of a macromolecular sample are typically measured by gel permeation or size exclusion chromatography using refractive index, UV, and/or light scattering detectors. Reference standards may be used to calibrate the chromatograph.

The term “polymerizable compound” means a compound containing one or more polymerizable groups. The term encompasses, for instance, monomers, macromers, oligomers, prepolymers, cross-linkers, and the like.

“Polymerizable groups” are groups that can undergo chain growth polymerization, such as free radical and/or ionic polymerization (e.g., cationic polymerization), for example a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyllactam, N-vinylamide, and styryl functional groups, and mixtures of any of the foregoing. More preferably, the free radical polymerizable groups comprise (meth)acrylates, (meth)acrylamides, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

Any type of free radical polymerization may be used including but not limited to bulk, solution, suspension, and emulsion as well as any of the controlled radical polymerization methods such as stable free radical polymerization, nitroxide-mediated living polymerization, atom transfer radical polymerization, reversible addition fragmentation chain transfer polymerization, organotellurium mediated living radical polymerization, and the like.

A “monomer” is a mono-functional molecule which can undergo chain growth polymerization, and in particular, undergo free radical polymerization, thereby creating a repeating unit in the chemical structure of the target macromolecule. A “repeating unit” is the smallest group of atoms in a polymer that corresponds to the polymerization of a specific monomer or macromer. Some monomers have di-functional impurities that can act as cross-linking agents. A “hydrophilic monomer” is a monomer which yields a clear single-phase solution when mixed with deionized water at 25° C. at a concentration of 5 weight percent, for instance, N, N-dimethyl acrylamide (DMA), N-vinylpyrrolidone (NVP), 2-hydroxyethyl methacrylate (HEMA), N-vinyl methacetamide (VMA), and N-vinyl N-methyl acetamide (NVA). A “hydrophilic component” is a monomer, macromer, prepolymer, initiator, cross-linker, additive, or polymer which yields a clear single-phase solution when mixed with deionized water at 25° C. at a concentration of 5 weight percent. A “hydrophobic component” is a monomer, macromer, prepolymer, initiator, cross-linker, additive, or polymer which is slightly soluble or insoluble in deionized water at 25° C.

A “macromonomer” or “macromer” is a macromolecule that has one group that can undergo chain growth polymerization, and in particular, undergo free radical polymerization, thereby creating a repeating unit in the chemical structure of the target macromolecule. Typically, the chemical structure of the macromer is different than the chemical structure of the target macromolecule, that is, the repeating unit of the macromer's pendent group is different than the repeating unit of the target macromolecule or its mainchain. The difference between a monomer and a macromer is merely one of chemical structure, molecular weight, and molecular weight distribution of the pendent group. As a result, and as used herein, the patent literature occasionally defines monomers as polymerizable compounds having relatively low molecular weights of about 1,500 Daltons or less, which inherently includes some macromers. In particular, monomethacryloxypropyl terminated mono-n-butyl terminated polydimethylsiloxane (number average molecular weight=500-1500 g/mol) (mPDMS) and mono-(2-hydroxy-3-methacryloxypropyl)-propyl ether terminated mono-n-butyl terminated polydimethylsiloxane (number average molecular weight=500-1500 g/mol) (OH-mPDMS) may be referred to as monomers or macromers. Furthermore, the patent literature occasionally defines macromers as having one or more polymerizable groups, essentially broadening the common definition of macromer to include prepolymers. As a result and as used herein, di-functional and multi-functional macromers, prepolymers, and cross-linkers may be used interchangeably.

A “polymer” is a target macromolecule composed of the repeating units of the monomers used during polymerization. A “homopolymer” is a polymer made from one monomer; a “copolymer” is a polymer made from two or more monomers; a “terpolymer” is a polymer made from three monomers. A “block copolymer” is composed of compositionally different blocks or segments. Diblock copolymers have two blocks. Triblock copolymers have three blocks. “Comb or graft copolymers” are made from at least one macromer.

An “initiator” or “free radical polymerization initiator” is a molecule that can decompose into radicals which can subsequently react with a monomer to initiate a free radical polymerization reaction. A “thermal initiator” decomposes at a certain rate depending on the temperature; typical examples are azo compounds such as 1,1′-azobisisobutyronitrile and 4,4′-azobis(4-cyanovaleric acid), peroxides such as benzoyl peroxide, tert-butyl peroxide, tert-butyl hydroperoxide, tert-butyl peroxybenzoate, dicumyl peroxide, and lauroyl peroxide, peracids such as peracetic acid and potassium persulfate as well as various redox systems. A “photo-initiator” decomposes by a photochemical process; typical examples are derivatives of benzil, benzoin, acetophenone, benzophenone, camphorquinone, and mixtures thereof as well as various monoacyl and bisacyl phosphine oxides and combinations thereof.

A “cross-linking agent” is a di-functional or multi-functional monomer or macromer which can undergo free radical polymerization at two or more locations on the molecule, thereby creating branch points and a polymeric network. Common examples are ethylene glycol dimethacrylate, tetraethylene glycol dimethacrylate, trimethylolpropane trimethacrylate, methylene bisacrylamide, triallyl cyanurate, and the like.

A “prepolymer” is a reaction product of monomers which contains remaining polymerizable groups capable of undergoing further reaction to form a polymer.

The term “multi-functional” refers to a component having two or more polymerizable groups. The term “mono-functional” refers to a component having one polymerizable group.

As used herein, the term “(meth)” designates optional methyl substitution. Thus, a term such as “(meth)acrylates” denotes both methacrylates and acrylates.

Wherever chemical structures are given, it should be appreciated that alternatives disclosed for the substituents on the structure may be combined in any combination. Thus, if a structure contained substituents R* and R**, each of which contained lists of three potential groups, then 9 combinations are disclosed. The same applies for combinations of properties.

When a subscript, such as “n” in the generic formula [***]n, is used to depict the number of repeating units in a polymer's chemical formula, the formula should be interpreted to represent the number average molecular weight of the macromolecule.

Unless otherwise indicated, ratios, percentages, parts, and the like are by weight.

Unless otherwise indicated, numeric ranges, for instance as in “from 2 to 10,” are inclusive of the numbers defining the range (e.g., 2 and 10).

The terms “reactive mixture” and “reactive monomer mixture” refer to the mixture of components (both retained and non-retained) which are mixed together and, when subjected to polymerization conditions, result in formation of a polymeric network as well as biomedical devices, ophthalmic devices, intraocular implants, contact lenses, and intraocular lenses made therefrom. The reactive mixture may comprise retained components such as monomers, macromers, prepolymers, cross-linkers, and initiators, additives such as wetting agents, polymers, dyes, light absorbing compounds such as UV/HEV absorbers, pigments, photochromic compounds, pharmaceutical compounds, and/or nutraceutical compounds, any of which may be reactive or non-reactive but are capable of being retained within the resulting biomedical device. The reactive mixture may also contain non-retained components which are intended to be removed from the device prior to its use, such as diluents. It will be appreciated that a wide range of additives may be added based upon the biomedical device which is made and its intended use. Concentrations of components of the reactive mixture are expressed as weight percentages of all retained components in the reactive mixture, therefore excluding non-retained components such as diluent. When diluents are used, their concentrations are expressed as weight percentages based upon the amount of all components in the reactive mixture (including the diluent).

“Reactive components” are the components in the reactive monomer mixture which become part of the structure of the polymeric network of the resulting composition. Diluents and processing aids which do not become part of the structure of the polymer are not reactive components.

“Retained components” are the polymerizable compounds (such as monomers, macromers, oligomers, prepolymers, and cross-linkers) in the reactive mixture, as well as any other components in the reactive mixture which are intended to substantially remain in the polymeric network after polymerization and all work-up steps (such as extraction steps) and packaging steps have been completed. Retained components may be retained in the polymeric network by covalent bonding, hydrogen bonding, electrostatic interactions, the formation of interpenetrating polymeric networks, or any other means. Components that are intended to release from the biomedical device once it is in use are still considered “retained components.” For example, pharmaceutical or nutraceutical components in a contact lens which are intended to be released during wear are considered “retained components.” Components that are intended to be removed from the polymeric network during the manufacturing process (e.g., by extraction), such as diluents, are “non-retained components.”

“Alkyl” or “aliphatic” are used interchangeably herein and refer to an optionally substituted linear or branched alkyl group containing the indicated number of carbon atoms. If no number is indicated, then alkyl (including any optional substituents on alkyl) may contain any of 1 to 24 carbon atoms, including 1, 2, 3, 4, 5, 6, 7, 8, 9, 10, 11, 12, 13, 14, 15, 16, 17, 18, 19, 20, 21, 22, 23, and 24 carbon atoms. Preferably, the alkyl group contains 1 to 18 carbon atoms, including 1, 2, 3, 4, 5, 6, 7, 8, 9, 10, 11, 12, 13, 14, 15, 16, 17, and 18 carbon atoms. Examples of alkyl include methyl, ethyl, propyl, isopropyl, butyl, iso-, sec- and tert-butyl, pentyl, hexyl, heptyl, 3-ethylbutyl, and the like. Examples of substituents on alkyl include 1, 2, or 3 groups independently selected from hydroxy, amino, amido, oxa, carboxy, alkyl carboxy, carbonyl, alkoxy, thioalkyl, carbamate, carbonate, halogen, phenyl, benzyl, and combinations thereof “Alkylene” means a divalent alkyl group, such as —CH₂—, —CH₂CH₂—, —CH₂CH₂CH₂—, —CH₂CH(CH₃)CH₂—, and —CH₂CH₂CH₂CH₂—.

“Amide” or “amido” refers to a moiety with formula —C(═O)NRR′ or —NRC(═O)R′, where R and R′ are each independently selected from the group consisting of hydrogen and alkyl. When the amido moiety is —C(═O)NRR′, R and R′ may optionally be taken together with the nitrogen to which they are attached to form a 4-, 5-, 6-, or 7-membered ring.

“Amidoalkyl” refers to an alkyl group as defined above substituted with one or more amido groups. Preferred amidoalkyl groups contain 1-6 carbons, 1-4 carbons, or 1-2 carbons.

The terms “halogen” or “halo” indicate fluorine, chlorine, bromine, and iodine. A preferred halogen is F.

“Haloalkyl” refers to an alkyl group as defined above substituted with one or more halogen atoms, where each halogen is independently F, Cl, Br or I. A preferred halogen is F. Preferred haloalkyl groups contain 1-6 carbons, 1-4 carbons, or 1-2 carbons. “Haloalkyl” includes perhaloalkyl groups, in which each hydrogen atom of the alkyl group is replaced with a halogen atom, such as —CF₃ or —CF₂CF₃. “Haloalkylene” means a divalent haloalkyl group, such as —CH₂CF₂— and —CF₂CF₂—.

“Hydroxy” refers to an —OH group.

“Hydroxyalkyl” refers to an alkyl group, as defined herein, substituted with at least one hydroxy group. Representative examples of hydroxyalkyl include, but are not limited to, hydroxymethyl, 2-hydroxyethyl, 3-hydroxypropyl, 2,3-dihydroxypropyl, 2,3-dihydroxypentyl, 4-hydroxybutyl, 2-ethyl-4-hydroxyheptyl, 3,4-dihydroxybutyl, and 5-hydroxypentyl.

“Cycloalkyl” or “cycloaliphatic” are used interchangeably herein and refer to an optionally substituted cyclic hydrocarbon containing the indicated number of ring carbon atoms. If no number is indicated, then cycloalkyl may contain 3 to 20 ring carbon atoms (e.g., 3 to 12 ring carbon atoms). Cycloaliphatic groups can be monocyclic, bicyclic, tricyclic, bridged, fused, and/or spirocyclic. Cycloaliphatic groups can also have one or more double bonds, provided that the group is not fully aromatic. Preferred monocyclic cycloaliphatic groups are C₃-C₈ cycloalkyl groups, C₃-C₇ cycloalkyl, more preferably C₄-C₇ cycloalkyl, and still more preferably C₅-C₆ cycloalkyl. Examples of cycloalkyl include cyclopropyl, cyclobutyl, cyclopentyl, cyclohexyl, cycloheptyl and cyclooctyl. Examples of substituents on cycloalkyl include 1, 2, or 3 groups independently selected from alkyl, hydroxy, amino, amido, oxa, carbonyl, alkoxy, thioalkyl, amido, carbamate, carbonate, halo, phenyl, benzyl, and combinations thereof “Cycloalkylene” means a divalent cycloalkyl group, such as 1,2-cyclohexylene, 1,3-cyclohexylene, or 1,4-cyclohexylene. “Cycloalkyl(alkyl)” groups mean alkyl groups as previously defined with at least one cycloalkyl substituent, such as cyclohexylmethyl, cyclohexylethyl, and cyclohexylpropyl.

“Heterocycloalkyl” refers to a cycloalkyl ring or ring system as defined above in which at least one ring carbon has been replaced with a heteroatom selected from nitrogen, oxygen, and sulfur. The heterocycloalkyl ring is optionally fused to or otherwise attached to other heterocycloalkyl rings and/or non-aromatic hydrocarbon rings and/or phenyl rings. Preferred heterocycloalkyl groups have from 5 to 7 members. More preferred heterocycloalkyl groups have 5 or 6 members. “Heterocycloalkylene” means a divalent heterocycloalkyl group.

“Aryl” refers to an optionally substituted aromatic hydrocarbon ring system containing at least one aromatic ring. The aryl group contains the indicated number of ring carbon atoms. If no number is indicated, then aryl may contain 6 to 14 ring carbon atoms. The aromatic ring may optionally be fused or otherwise attached to other aromatic hydrocarbon rings or non-aromatic hydrocarbon rings. Examples of aryl groups include phenyl, naphthyl, and biphenyl. Preferred examples of aryl groups include phenyl. Examples of substituents on aryl include 1, 2, or 3 groups independently selected from alkyl, hydroxy, amino, amido, oxa, carboxy, alkyl carboxy, carbonyl, alkoxy, thioalkyl, carbamate, carbonate, halo, phenyl, benzyl, and combinations thereof. “Arylene” means a divalent aryl group, for example 1,2-phenylene, 1,3-phenylene, or 1,4-phenylene.

“Arylalkyl” refers to an aryl group, as defined herein, appended to the parent molecular moiety through an alkyl group, as defined herein. Representative examples of arylalkyl include phenylmethyl (i.e., benzyl), phenylethyl, and phenylpropyl.

“Heteroaryl” refers to an aryl ring or ring system, as defined above, in which at least one ring carbon atom has been replaced with a heteroatom selected from nitrogen, oxygen, and sulfur. The heteroaryl ring may be fused or otherwise attached to one or more heteroaryl rings, aromatic or nonaromatic hydrocarbon rings or heterocycloalkyl rings. Examples of heteroaryl groups include pyridyl, furyl, and thienyl. “Heteroarylene” means a divalent heteroaryl group.

“Heteroarylalkyl” refers to a heteroaryl group, as defined herein, appended to the parent molecular moiety through an alkyl group, as defined herein. Representative examples of arylalkyl include thiophen-2-ylmethyl, furan-2-ylmethyl, and pyridylmethyl.

“Alkoxy” refers to an alkyl group attached to the parent molecular moiety through an oxygen bridge. Examples of alkoxy groups include, for instance, methoxy, ethoxy, propoxy and isopropoxy. “Thioalkyl” means an alkyl group attached to the parent molecule through a sulfur bridge. Examples of thioalkyl groups include, for instance, methylthio, ethylthio, n-propylthio and iso-propylthio. “Aryloxy” refers to an aryl group attached to a parent molecular moiety through an oxygen bridge. Examples include phenoxy. “Arylthio” refers to an aryl group attached to a parent molecular moiety through a sulfur bridge. Examples include phenylthiol. “Cyclic alkoxy” means a cycloalkyl group attached to the parent moiety through an oxygen bridge.

“Alkoxyalkyl” refers to an alkyl group, as defined herein, substituted with at least one alkoxy group, as defined herein. Representative examples of alkoxyalkyl include, but are not limited to, methoxymethyl, 2-methoxyethyl, and 3-methoxypropyl.

Alkylamine” refers to an alkyl group attached to the parent molecular moiety through an —NH bridge. Alkyleneamine means a divalent alkylamine group, such as —CH₂CH₂NH—.

“Alkyleneoxy” refers to groups of the general formula (alkylene-O)ₚ— or —(O-alkylene)ₚ-, wherein alkylene is as defined above, and p is from 1 to 200, or from 1 to 100, or from 1 to 50, or from 1 to 25, or from 1 to 20, or from 1 to 10, wherein each alkylene is independently optionally substituted with one or more groups independently selected from hydroxyl, halo (e.g., fluoro), amino, amido, ether, carbonyl, carboxyl, and combinations thereof. If p is greater than 1, then each alkylene may be the same or different and the alkyleneoxy may be in block or random configuration. When alkyleneoxy forms a terminal group in a molecule, the terminal end of the alkyleneoxy may, for instance, be a hydroxy or alkoxy (e.g., HO—[CH₂CH₂O]ₚ— or CH₃O—[CH₂CH₂O]ₚ—). Examples of alkyleneoxy include polyethyleneoxy, polypropyleneoxy, polybutyleneoxy, and poly(ethyleneoxy-co-propyleneoxy).

“Oxaalkylene” refers to an alkylene group as defined above where one or more non-adjacent CH₂ groups have been substituted with an oxygen atom, such as —CH₂CH₂OCH(CH₃)CH₂—. “Thiaalkylene” refers to an alkylene group as defined above where one or more non-adjacent CH₂ groups have been substituted with a sulfur atom, such as —CH₂CH₂SCH(CH₃)CH₂—.

The term “linking group” refers to a moiety that links a polymerizable group to the parent molecule. The linking group may be any moiety that is compatible with the compound of which it is a part, and that does not undesirably interfere with the polymerization of the compound, is stable under the polymerization conditions as well as the conditions for the processing and storage of the final product. For instance, the linking group may be a bond, or it may comprise one or more alkylene, haloalkylene, amide, amine, alkyleneamine, carbamate, ester (—CO₂—), arylene, heteroarylene, cycloalkylene, heterocycloalkylene, alkyleneoxy, oxaalkylene, thiaalkylene, haloalkyleneoxy (alkyleneoxy substituted with one or more halo groups, e.g., —OCF₂—, —OCF₂CF₂—, —OCF₂CH₂—), siloxanyl, alkylenesiloxanyl, or combinations thereof. The linking group may optionally be substituted with 1 or more substituent groups. Suitable substituent groups may include those independently selected from alkyl, halo (e.g., fluoro), hydroxyl, HO-alkyleneoxy, MeO-alkyleneoxy, siloxanyl, siloxy, siloxy-alkyleneoxy-, siloxy-alkylene-alkyleneoxy- (where more than one alkyleneoxy groups may be present and wherein each methylene in alkylene and alkyleneoxy is independently optionally substituted with hydroxyl), ether, amine, carbonyl, carbamate, and combinations thereof. The linking group may also be substituted with a polymerizable group, such as (meth)acrylate (in addition to the polymerizable group to which the linking group is linked).

Preferred linking groups include C₁-C₈ alkylene (preferably C₂-C₆ alkylene) and C₁-C₈ oxaalkylene (preferably C₂-C₆ oxaalkylene), each of which is optionally substituted with 1 or 2 groups independently selected from hydroxyl and siloxy. Preferred linking groups also include ester, amide, C₁-C₈ alkylene-ester-C₁-C₈ alkylene, or C₁-C₈ alkylene-amide-C₁-C₈ alkylene.

The term “electron withdrawing group” (EWG) refers to a chemical group which withdraws electron density from the atom or group of atoms to which the electron withdrawing group is attached. Examples of EWGs include, but are not limited to, cyano, amide, ester, keto, or aldehyde.

When the compounds described herein contain olefinic double bonds or other centers of geometric asymmetry, and unless otherwise specified, it is intended that the compounds include the cis, trans, Z- and E-configurations. Likewise, all tautomeric and salt forms are also intended to be included.

The terms “light absorbing compound” refers to a chemical material that absorbs light within the visible spectrum (e.g., in the 380 nanometer to 780 nanometer range). A “high energy radiation absorber,” “UV/HEV absorber,” “UV/HEV absorbing compound,” or “high energy light absorbing compound” is a chemical material that absorbs various wavelengths of ultraviolet light, high energy visible light, or both. A material's ability to absorb certain wavelengths of light can be determined by measuring its UV/VIS transmission spectrum. Compounds that exhibit no absorption at a particular wavelength will exhibit substantially 100 percent transmission at that wavelength. Conversely, compounds that completely absorb at a particular wavelength will exhibit substantially 0% transmission at that wavelength.

“Silyl” refers to a structure of formula R₃Si— and “siloxy” refers to a structure of formula R₃Si—O—, where each R in silyl or siloxy is independently selected from trimethylsiloxy, C₁-C₈ alkyl (preferably C₁-C₃ alkyl, more preferably methyl or ethyl), and C₃-C₈ cycloalkyl.

“Siloxanyl” refers to a structure having at least one Si—O—Si bond. Thus, for example, siloxanyl group means a group having at least one Si—O—Si group (i.e. a siloxane group), and siloxanyl compound means a compound having at least one Si—O—Si group. “Siloxanyl” encompasses monomeric (e.g., Si—O—Si) as well as oligomeric/polymeric structures (e.g., [Si—O]ₙ, where n is 2 or more). Each silicon atom in the siloxanyl group is substituted with independently selected R^{A} groups (where R^{A} is as defined in Formula A options (b)-(i)) to complete their valence.

A “silicone-containing component” may comprise one or more polymerizable compounds of Formula A:

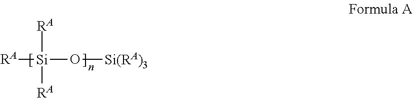

wherein at least one R^{A} is a group of Formula P_{g}-L-, wherein P_{g} is a polymerizable group and L is a linking group, and the remaining R^{A} are each independently
a) P_{g}-L-
b) C₁-C₁₆ alkyl optionally substituted with one or more hydroxy, amino, amido, oxa, carboxy, alkyl carboxy, carbonyl, alkoxy, amido, carbamate, carbonate, halo, phenyl, benzyl, or combinations thereof
c) C₃-C₁₂ cycloalkyl optionally substituted with one or more alkyl, hydroxy, amino, amido, oxa, carbonyl, alkoxy, amido, carbamate, carbonate, halo, phenyl, benzyl, or combinations thereof
d) a C₆-C₁₄ aryl optionally substituted with one or more alkyl, hydroxy, amino, amido, oxa, carboxy, alkyl carboxy, carbonyl, alkoxy, amido, carbamate, carbonate, halo, phenyl, benzyl, or combinations thereof
e) halo
f) alkoxy, cyclic alkoxy, or aryloxy
g) siloxy
h) alkyleneoxy-alkyl or alkoxy-alkyleneoxy-alkyl, such as polyethyleneoxyalkyl, polypropyleneoxyalkyl, or poly(ethyleneoxy-co-propyleneoxyalkyl), or
i) a monovalent siloxane chain comprising from 1 to 100 siloxane repeating units optionally substituted with alkyl, alkoxy, hydroxy, amino, oxa, carboxy, alkyl carboxy, alkoxy, amido, carbamate, halo or combinations thereof
   wherein n is from 0 to 500 or from 0 to 200, or from 0 to 100, or from 0 to 20, where it is understood that when n is other than 0, n is a distribution having a mode equal to a stated value. When n is 2 or more, the SiO units may carry the same or different R^{A} substituents and if different R^{A} substituents are present, the n groups may be in random or block configuration.

In Formula A, three R^{A} may each comprise a polymerizable group, alternatively two R^{A} may each comprise a polymerizable group, or alternatively one R^{A} may comprise a polymerizable group.

When the linking group is comprised of combinations of moieties as described above (e.g., alkylene and cycloalkylene), the moieties may be present in any order. For instance, if in Formula A above, L is indicated as being -alkylene-cycloalkylene-, then P_{g}-L may be either P_{g}-alkylene-cycloalkylene-, or P_{g}-cycloalkylene-alkylene-. Notwithstanding this, the listing order represents the preferred order in which the moieties appear in the compound starting from the terminal polymerizable group (P_{g}) to which the linking group is attached. For example, if in Formula A, L is indicated as being alkylene-cycloalkylene, then P_{g}-L is preferably P_{g}-alkylene-cycloalkylene-. Some preferred silicone-containing components are mono-n-butyl terminated monomethacryloxypropyl terminated polydimethylsiloxane (mPDMS), mono-n-butyl terminated mono-(2-hydroxy-3-methacryloxypropyloxy)-propyl terminated polydimethylsiloxane (OH-mPDMS), 3-(3-(1,1,1,3,5,5,5-heptamethyltrisiloxan-3-yl)propoxy)-2-hydroxypropyl methacrylate (SiMAA), and 3-(3-(1,5-di-tert-butyl-1,1,3,5,5-pentamethyltrisiloxan-3-yl)propoxy)-2-hydroxypropyl methacrylate (tBu-SiMAA).

The term “optional substituent” means that a hydrogen atom in the underlying moiety is optionally replaced by a substituent. Any substituent may be used that is sterically practical at the substitution site and is synthetically feasible. Identification of a suitable optional substituent is well within the capabilities of an ordinarily skilled artisan. Examples of an “optional substituent” include, without limitation, C₁-C₆ alkyl, C₁-C₆ alkoxy, C₁-C₆ thioalkyl, C₃-C₇ cycloalkyl, aryl, halo, hydroxy, amino, NR′R″, benzyl, SO₃H, or SO₃Na, wherein R′ and R″ are independently H or C₁-C₆ alkyl. The foregoing substituents may be optionally substituted by an optional substituent (which, unless otherwise indicated, is preferably not further substituted). For instance, alkyl may be substituted by halo (resulting, for instance, in CF₃).

In some embodiments, the reactive monomer mixture includes at least one polyamide. As used herein, the term “polyamide” refers to polymers and copolymers comprising repeating units containing amide groups. The polyamide may comprise cyclic amide groups, acyclic amide groups and combinations thereof, and may be any polyamide known to those of skill in the art. Acyclic polyamides comprise pendant acyclic amide groups and are capable of association with hydroxyl groups. Cyclic polyamides comprise cyclic amide groups and are capable of association with hydroxyl groups. Polyamides suitable for use with the presently disclosed compositions and methods are disclosed in U.S. Patent Application Publication No. 20180009922 for SILICONE HYDROGELS COMPRISING HIGH LEVELS OF POLYAMIDES to Alli et al., published Jan. 11, 2018, and U.S. Patent Application Publication No. 20180011222 for SILICONE HYDROGELS COMPRISING POLYAMIDES to Alli et al., published Jan. 11, 2018, each of which are incorporated herein by reference in their entirety. Some preferred polyamides are polyvinylpyrrolidone (PVP), polyvinylmethyacetamide (PVMA), polydimethylacrylamide (PDMA), polyvinylacetamide (PNVA), and combinations thereof.

The term “individual” includes humans and non-human vertebrates.

The term “biomedical device” refers to any article that is designed to be used while either in or on mammalian tissues or fluids, and preferably in or on human tissue or fluids. Examples of these devices include but are not limited to wound dressings, sealants, tissue fillers, drug delivery systems, coatings, adhesion prevention barriers, catheters, implants, stents, and ophthalmic devices such as intraocular implants, intraocular lenses, and contact lenses. The biomedical devices may be ophthalmic devices, particularly ophthalmic implants or ophthalmic lenses made from the reactive monomer compositions described herein.

The term “ocular surface” includes the surface and glandular epithelia of the cornea, conjunctiva, lacrimal gland, accessory lacrimal glands, nasolacrimal duct and meibomian gland, and their apical and basal matrices, puncta and adjacent or related structures, including eyelids linked as a functional system by both continuity of epithelia, by innervation, and the endocrine and immune systems.

The term “ophthalmic device” refers to any device which resides in or on the eye or any part of the eye, including the ocular surface. These devices can provide optical correction, cosmetic enhancement, vision enhancement, therapeutic benefit (for example as bandages) or delivery of active components such as pharmaceutical and nutraceutical components, or a combination of any of the foregoing. Examples of ophthalmic devices include but are not limited to lenses, optical and ocular inserts, including but not limited to punctal plugs, and the like. “Lenses” include soft contact lenses, hard contact lenses, hybrid contact lenses, intraocular lenses, and overlay lenses. The ophthalmic device may comprise an intraocular implant, intraocular lens, or contact lens.

The term “contact lens” refers to an ophthalmic device that can be placed on the cornea of an individual's eye. The contact lens may provide corrective, cosmetic, or therapeutic benefit, including wound healing, the delivery of drugs or nutraceuticals, diagnostic evaluation or monitoring, ultraviolet (UV) light absorbing, visible (VIS) light or glare reduction, or any combination thereof. A contact lens can be of any appropriate material known in the art and can be a soft lens, a hard lens, or a hybrid lens containing at least two distinct portions with different physical, mechanical, or optical properties, such as modulus, water content, light transmission, or combinations thereof.

“Intraocular lens” refers to a lens implanted in an eye. In some embodiments, the intraocular lens is implanted in the eye to replace an existing crystalline lens (such as, for example, because the existing lens has been clouded over by a cataract, or as a form of refractive surgery to change the eye's optical power).

“Abbe number,” also known as the V-number or constringence of a transparent material, is a measure of the material's dispersion, i.e., variation of refractive index versus wavelength, with high values of V indicating low dispersion. The Abbe number of a material is defined by the formula: Abbe number V=(n_{D}−1)/(n_{F}−n_{C}), wherein n_{D}, n_{F} and n_{C} are the refractive indices of the material at the wavelengths of the Fraunhofer D, F and C spectral lines (589.3 nanometers, 486.1 nanometers and 656.3 nanometers, respectively).

“Refractive index” for a medium is defined by the formula: refractive index n=c/v, wherein c is the speed of light in a vacuum, and v is the phase velocity of light in the medium.

### B. Compositions

In some aspects of the invention, the presently disclosed subject matter provides a composition made by free radical polymerization of a reactive monomer mixture comprising:
(a) a compatibilizing monomer selected from the group consisting of a pendant carbamate monomer having a chemical structure of Formula I, a pendant amide monomer having a chemical structure of Formula II, and combinations thereof, having the chemical structures of Formula I, P_{g}-L-OCONR¹R², and Formula II, P_{g}-L-CONR′R², wherein P_{g} is a polymerizable group, L is a linking group, and R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, and heteroalkyl groups;
(b) a cross-linking agent; and
(c) an ethylene glycol dicyclopentenyl ether (meth)acrylate;
   wherein the concentration of the ethylene glycol dicyclopentenyl ether (meth)acrylate in the reactive monomer mixture excluding any diluent is greater than or equal to 20 weight percent; and wherein the composition exhibits a refractive index of at least 1.45 and an Abbe number of at least 39 [“Composition (A)” ].

Non-limiting examples of the polymerizable group of the compatibilizing monomer in Composition (A) include (meth)acrylates, (meth)acrylamides, N-vinyllactams, N-vinylamides, styrenes, vinyl ethers, O-vinylcarbamates, O-vinylcarbonates, and other vinyl groups. Preferably, the polymerizable groups comprise (meth)acrylate, (meth)acrylamide, N-vinyllactam, N-vinylamide groups, and mixtures thereof. More preferably, the polymerizable groups comprise (meth)acrylates, (meth)acrylamides, and combinations thereof. Most preferably, the polymerizable groups comprise (meth)acrylates.

Non-limiting examples of the linking group of the compatibilizing monomer in Composition (A) include alkylene, haloalkylene, amide, amine, alkyleneamine, carbamate, ester, arylene, heteroarylene, cycloalkylene, heterocycloalkylene, alkyleneoxy, oxaalkylene, thiaalkylene, and haloalkyleneoxy. Preferably, the linking groups comprise ester, amide, C₁-C₈ alkylene, C₁-C₈ oxaalkylene, C₁-C₈ alkylene-ester-C₁-C₈ alkylene, C₁-C₈ alkylene-amide-C₁-C₈ alkylene. More preferably, the linking groups comprise C₁-C₈ alkylene and C₁-C₈ oxaalkylene. Most preferably, the linking groups comprise C₁-C₈ alkylene. Especially preferred linking groups are unsubstituted C₁-C₄ alkylene.

In a specific aspect of the invention, the compatibilizing monomer in Composition (A) is a pendant carbamate monomer having a chemical structure shown in Formula III:

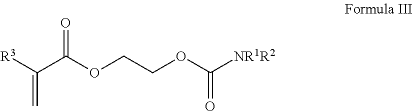

wherein R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups, and R³ is H or methyl.

In another specific aspect of the invention, the compatibilizing monomer in Composition (A) is a pendant amide monomer having a chemical structure shown in Formula IV:

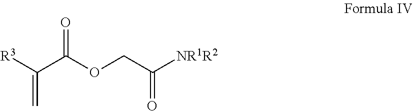

wherein R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups, and R³ is H or methyl.

In yet another specific aspect of the invention, the compatibilizing monomer in Composition (A) is a mixture of pendant carbamate monomers and pendant amide monomers.

Non-limiting examples of R¹ and R² independently in Formulae III and IV are hydrogen, C₁-C₂₄ linear alkyl groups, C₁-C₂₄ branched alkyl groups, C₃-C₂₀ cycloalkyl, cycloalkyl(alkyl) groups in which the cycloalkyl is C₃-C₂₀ cycloalkyl and the alkyl is C₁-C₂₄ linear alkyl groups or C₁-C₂₄ branched alkyl groups, and combinations thereof, optionally substituted with hydroxy, alkoxy, or halogen. Preferably, R¹ and R² are independently selected from hydrogen, C₁-C₁₅ linear alkyl groups, C₁-C₁₅ branched alkyl groups, or mixtures thereof, optionally substituted with hydroxy, alkoxy, or halogen. More preferably, R¹ and R² are independently selected from hydrogen, unsubstituted C₁-C₁₅ linear alkyl groups, unsubstituted C₁-C₁₅ branched alkyl groups, or mixtures thereof. Also preferably, R¹ and R² are independently selected from unsubstituted C₁-C₆ alkyl, or are independently selected from unsubstituted C₁-C₃ alkyl. Further preferably, R¹ and R² are both methyl. Most preferably, R¹ is a hydrogen and R² is an unsubstituted C₁-C₁₅ linear alkyl group.

In one aspect of the invention, the compatibilizing monomer of Composition (A) is a pendant carbamate monomer selected from the group consisting of 2-((methylcarbamoyl)oxy)ethyl (meth)acrylate, 2-((ethylcarbamoyl)oxy)ethyl (meth)acrylate, 2-((propylcarbamoyl)oxy)ethyl (meth)acrylate, 2-((butylcarbamoyl)oxy)ethyl (meth)acrylate, 2-((pentylcarbamoyl)oxy)ethyl (meth)acrylate, 2-((hexylcarbamoyl)oxy)ethyl (meth)acrylate, 2-((heptylcarbamoyl)oxy)ethyl (meth)acrylate, 2-((octylcarbamoyl)oxy)ethyl (meth)acrylate, 2-((nonylcarbamoyl)oxy)ethyl (meth)acrylate, 2-((decylcarbamoyl)oxy)ethyl (meth)acrylate, 2-((undecylcarbamoyl)oxy)ethyl (meth)acrylate, 2-((dodecylcarbamoyl)oxy)ethyl (meth)acrylate, 2-((tridecylcarbamoyl)oxy)ethyl (meth)acrylate, 2-((tetradecylcarbamoyl)oxy)ethyl (meth)acrylate, 2-((pentadecylcarbamoyl)oxy)ethyl (meth)acrylate, 2-((hexadecylcarbamoyl)oxy)ethyl (meth)acrylate, 2-((heptadecylcarbamoyl)oxy)ethyl (meth)acrylate, and combinations thereof. Preferably, the pendant carbamate monomer is selected from 2-((methylcarbamoyl)oxy)ethyl acrylate, 2-((ethylcarbamoyl)oxy)ethyl acrylate, 2-((propylcarbamoyl)oxy)ethyl acrylate, 2-((butylcarbamoyl)oxy)ethyl acrylate, 2-((pentylcarbamoyl)oxy)ethyl acrylate, 2-((hexylcarbamoyl)oxy)ethyl acrylate, 2-((heptylcarbamoyl)oxy)ethyl acrylate, 2-((octylcarbamoyl)oxy)ethyl acrylate, 2-((nonylcarbamoyl)oxy)ethyl acrylate, 2-((decylcarbamoyl)oxy)ethyl acrylate, 2-((undecylcarbamoyl)oxy)ethyl acrylate, 2-((dodecylcarbamoyl)oxy)ethyl acrylate, 2-((tridecylcarbamoyl)oxy)ethyl acrylate, 2-((tetradecylcarbamoyl)oxy)ethyl acrylate, 2-((pentadecylcarbamoyl)oxy)ethyl acrylate, 2-((hexadecylcarbamoyl)oxy)ethyl acrylate, 2-((heptadecylcarbamoyl)oxy)ethyl acrylate, and combinations thereof. More preferably, the pendant carbamate monomer is selected from 2-((methylcarbamoyl)oxy)ethyl acrylate, 2-((ethylcarbamoyl)oxy)ethyl acrylate, 2-((propylcarbamoyl)oxy)ethyl acrylate, 2-((butylcarbamoyl)oxy)ethyl acrylate, 2-((pentylcarbamoyl)oxy)ethyl acrylate, 2-((hexylcarbamoyl)oxy)ethyl acrylate, 2-((heptylcarbamoyl)oxy)ethyl acrylate, 2-((octylcarbamoyl)oxy)ethyl acrylate, 2-((nonylcarbamoyl)oxy)ethyl acrylate, 2-((decylcarbamoyl)oxy)ethyl acrylate, and combinations thereof. Most preferably, the pendant carbamate monomer is selected from 2-((butylcarbamoyl)oxy)ethyl acrylate, 2-((pentylcarbamoyl)oxy)ethyl acrylate, 2-((hexylcarbamoyl)oxy)ethyl acrylate, 2-((heptylcarbamoyl)oxy)ethyl acrylate, 2-((octylcarbamoyl)oxy)ethyl acrylate, 2-((nonylcarbamoyl)oxy)ethyl acrylate, 2-((decylcarbamoyl)oxy)ethyl acrylate, and combinations thereof. An especially preferred pendant carbamate monomer is 2-((butylcarbamoyl)oxy)ethyl acrylate.

In another aspect of the invention, the compatibilizing monomer of Composition (A) is a pendant amide monomer selected from the group consisting of 2-oxo-2-(methylamino)ethyl (meth)acrylate, 2-oxo-2-(ethylamino)ethyl (meth)acrylate, 2-oxo-2-(propylamino)ethyl (meth)acrylate, 2-oxo-2-(butylamino)ethyl (meth)acrylate, 2-oxo-2-(pentylamino)ethyl (meth)acrylate, 2-oxo-2-(hexylamino)ethyl (meth)acrylate, 2-oxo-2-(heptylamino)ethyl (meth)acrylate, 2-oxo-2-(octylamino)ethyl (meth)acrylate, 2-oxo-2-(nonylamino)ethyl (meth)acrylate, 2-oxo-2-(decylamino)ethyl (meth)acrylate, 2-oxo-2-(undecylamino)ethyl (meth)acrylate, 2-oxo-2-(dodecylamino)ethyl (meth)acrylate, 2-oxo-2-(tridecylamino)ethyl (meth)acrylate, 2-oxo-2-(tetradecylamino)ethyl (meth)acrylate, 2-oxo-2-((3-methoxypropyl)amino)ethyl (meth)acrylate, 2-oxo-2-((3-ethoxypropyl)amino)ethyl (meth)acrylate, 2-oxo-2-((cyclohexylmethyl)amino)ethyl (meth)acrylate, 2-oxo-2-(benzylamino)ethyl (meth)acrylate, 2-oxo-2-(phenethylamino)ethyl (meth)acrylate, 2-oxo-2-((thiophen-2-ylmethyl)amino)ethyl (meth)acrylate, 2-oxo-2-((2,3-dihydroxypropyl)amino)ethyl (meth)acrylate, and combinations thereof. Preferably, the pendant amide monomer is selected from 2-oxo-2-(methylamino)ethyl acrylate, 2-oxo-2-(ethylamino)ethyl acrylate, 2-oxo-2-(propylamino)ethyl acrylate, 2-oxo-2-(butylamino)ethyl acrylate, 2-oxo-2-(pentylamino)ethyl acrylate, 2-oxo-2-(hexylamino)ethyl acrylate, 2-oxo-2-(heptylamino)ethyl acrylate, 2-oxo-2-(octylamino)ethyl acrylate, 2-oxo-2-(nonylamino)ethyl acrylate, 2-oxo-2-(decylamino)ethyl acrylate, 2-oxo-2-(undecylamino)ethyl acrylate, 2-oxo-2-(dodecylamino)ethyl acrylate, 2-oxo-2-(tridecylamino)ethyl acrylate, 2-oxo-2-(tetradecylamino)ethyl acrylate, 2-oxo-2-((3-methoxypropyl)amino)ethyl acrylate, 2-oxo-2-((3-ethoxypropyl)amino)ethyl acrylate, 2-oxo-2-((cyclohexylmethyl)amino)ethyl acrylate, 2-oxo-2-(benzylamino)ethyl acrylate, 2-oxo-2-(phenethylamino)ethyl acrylate, 2-oxo-2-((thiophen-2-ylmethyl)amino)ethyl acrylate, 2-oxo-2-((2,3-dihydroxypropyl)amino)ethyl acrylate, 2-(dimethylamino)-2-oxoethyl methacrylate, and combinations thereof. More preferably, the pendant amide monomer is selected from 2-oxo-2-(methylamino)ethyl acrylate, 2-oxo-2-(ethylamino)ethyl acrylate, 2-oxo-2-(propylamino)ethyl acrylate, 2-oxo-2-(butylamino)ethyl acrylate, 2-oxo-2-(pentylamino)ethyl acrylate, 2-oxo-2-(hexylamino)ethyl acrylate, 2-oxo-2-(heptylamino)ethyl acrylate, 2-oxo-2-(octylamino)ethyl acrylate, 2-oxo-2-(nonylamino)ethyl acrylate, 2-oxo-2-(decylamino)ethyl acrylate, and combinations thereof. Most preferably, the pendant amide monomer is 2-oxo-2-(pentylamino)ethyl acrylate, 2-oxo-2-(octylamino)ethyl acrylate, 2-oxo-2-(decylamino)ethyl acrylate, and combinations thereof.

When the compatibilizing monomer in Composition (A) is a mixture of pendant carbamate monomers and pendant amide monomers, a preferred mixture comprises any combination of 2-((butylcarbamoyl)oxy)ethyl (meth)acrylate, 2-oxo-2-(pentylamino)ethyl (meth)acrylate, 2-oxo-2-(octylamino)ethyl (meth)acrylate, and 2-oxo-2-(decylamino)ethyl (meth)acrylate. A more preferred mixture comprises a combination of 2-((butylcarbamoyl)oxy)ethyl acrylate and 2-oxo-2-(pentylamino)ethyl acrylate.

The reactive monomer mixture of Composition (A) may comprise a compatibilizing monomer in an amount between about 0.01 weight percent and about 55 weight percent, between about 1 weight percent and about 40 weight percent, between about 5 weight percent and about 35 weight percent, between about 10 weight percent and about 30 weight percent, or between about 20 weight percent and about 30 weight percent.

Non-limiting examples of the cross-linking agent in Composition (A) are tricyclo[5.2.1.0^{2,6}]decanedimethanol di(meth)acrylate, ethylene glycol di(meth)acrylate, diethylene glycol di(meth)acrylate, triethylene glycol di(meth)acrylate, tetraethylene glycol di(meth)acrylate, trimethylolpropane tri(meth)acrylate, 1,4-butanediol di(meth)acrylate, 1,5-pentanediol di(meth)acrylate, 1,6-hexanediol di(meth)acrylate, 1,7-heptanediol di(meth)acrylate, 1,8-octanediol di(meth)acrylate, 1,9-nonanediol di(meth)acrylate, 1,10-decanediol di(meth)acrylate, 1,11-undecanediol di(meth)acrylate, 1,12-dodecanediol di(meth)acrylate, 1,13-tridecanediol di(meth)acrylate, 1,14-tetradecanediol di(meth)acrylate, 1,15-pentadecanediol di(meth)acrylate, 1,16-hexadecanediol di(meth)acrylate, 1,17-heptadecanediol di(meth)acrylate, 1,18-octadecanediol di(meth)acrylate, glycerol tri(meth)acrylate, triallyl cyanurate, methylene bis(meth)acrylamide, poly(ethylene glycol) di(meth)acrylate, and any combination thereof. Preferably, the cross-linking agent is selected from tricyclo[5.2.1.0^{2,6}]decanedimethanol diacrylate, ethylene glycol dimethacrylate, tetraethylene dimethacrylate, and combinations thereof. More preferably, the cross-linking agent is tricyclo[5.2.1.0^{2,6}]decanedimethanol di(meth)acrylate. Most preferably, the cross-linking agent is tricyclo[5.2.1.0^{2,6}]decanedimethanol diacrylate.

The reactive monomer mixture of Composition (A) may comprise a cross-linking agent in an amount between about 0.1 weight percent and about 10 weight percent; between about 0.1 weight percent and about 5 weight percent; between about 0.5 weight percent and about 3 weight percent; or between about 1 weight percent and 3 weight percent.

The reactive monomer mixture of Composition (A) may comprise ethylene glycol dicyclopentenyl ether (meth)acrylate in an amount between about 25 weight percent and about 95 weight percent, between about 30 weight percent and about 75 weight percent, between about 40 weight percent and about 65 weight percent, or between about 45 weight percent and about 60 weight percent.

A preferred ethylene glycol dicyclopentenyl ether (meth)acrylate in Composition (A) is ethylene glycol dicyclopentenyl ether acrylate.

In some aspects of the invention, the reactive monomer mixture of Composition (A) further comprises an alkyl (meth)acrylate monomer, wherein the alkyl group contains between one and twenty carbon atoms. The alkyl group may be a linear alkyl group or a branched alkyl group. Preferably, the alkyl (meth)acrylate is selected from methyl (meth)acrylate, ethyl (meth)acrylate, n-propyl (meth)acrylate, 2-propyl (meth)acrylate, n-butyl (meth)acrylate, 2-butyl (meth)acrylate, tert-butyl (meth)acrylate, n-pentyl (meth)acrylate, 2-pentyl (meth)acrylate, 3-pentyl (meth)acrylate, n-hexyl (meth)acrylate, 2-hexyl (meth)acrylate, 3-hexyl (meth)acrylate, n-heptyl (meth)acrylate, n-octyl (meth)acrylate, 2-ethylhexyl (meth)acrylate, n-nonyl (meth)acrylate, n-decyl (meth)acrylate, n-undecyl (meth)acrylate, n-dodecyl (meth)acrylate, n-tridecyl (meth)acrylate, n-tetradecyl (meth)acrylate, n-pentadecyl (meth)acrylate, n-hexadecyl (meth)acrylate, n-heptadecyl (meth)acrylate, n-octadecyl methyl (meth)acrylate, and combinations thereof. More preferably, the alkyl (meth)acrylate is selected from n-propyl acrylate, n-butyl acrylate, n-pentyl acrylate, n-hexyl acrylate, n-heptyl acrylate, n-octyl acrylate, n-nonyl acrylate, n-decyl acrylate, n-undecyl acrylate, n-dodecyl acrylate, and combinations thereof. Most preferably, the alkyl (meth)acrylate is selected from n-pentyl acrylate, n-hexyl acrylate, n-heptyl acrylate, n-octyl acrylate, and combinations thereof. An especially preferred alkyl (meth)acrylate is n-hexyl acrylate.

The reactive monomer mixture of Composition (A) may comprise alkyl (meth)acrylate in an amount between about 0.01 and about 20 weight percent, between about 1 weight percent and 20 weight percent, between about 1 weight percent and about 15 weight percent, or between about 1 weight percent and about 10 weight percent.

In some aspects of the invention, the reactive monomer mixture of Composition (A) further comprises a hydroxyalkyl (meth)acrylate monomer, wherein the hydroxyalkyl group contains between one and twenty carbon atoms. The hydroxyalkyl group may be a linear hydroxyalkyl group or a branched hydroxyalkyl group. Preferably, the hydroxyalkyl (meth)acrylate is selected from 2-hydroxyethyl (meth)acrylate, 2-hydroxypropyl (meth)acrylate, 3-hydroxypropyl (meth)acrylate, 2,3-dihydroxypropyl (meth)acrylate, 1,1-dimethyl-2-hydroxyethyl (meth)acrylate, 2-hydroxybutyl (meth)acrylate, 3-hydroxybutyl (meth)acrylate 4-hydroxybutyl (meth)acrylate, 5-hydroxypentyl (meth)acrylate, 6-hydroxyhexyl (meth)acrylate, 7-hydroxyheptyl (meth)acrylate, 8-hydroxyoctyl (meth)acrylate, 2-ethyl-6-hydroxyhexyl (meth)acrylate, 9-hydroxynonyl (meth)acrylate, and 10-hydroxydecyl (meth)acrylate, and combinations thereof. More preferably, the hydroxyalkyl (meth)acrylate is selected from 2-hydroxyethyl acrylate, 2-hydroxypropyl acrylate, 3-hydroxypropyl acrylate, 2,3-dihydroxypropyl acrylate, 1,1-dimethyl-2-hydroxyethyl acrylate, 2-hydroxybutyl acrylate, 3-hydroxybutyl acrylate 4-hydroxybutyl acrylate, 5-hydroxypentyl acrylate, 6-hydroxyhexyl acrylate, 7-hydroxyheptyl acrylate, 8-hydroxyoctyl acrylate, 2-ethyl-6-hydroxyhexyl acrylate, 9-hydroxynonyl acrylate, and 10-hydroxydecyl acrylate, and combinations thereof. Most preferably, the hydroxyalkyl (meth)acrylate is selected from 2-hydroxyethyl acrylate, 2-hydroxypropyl acrylate, 3-hydroxypropyl acrylate, 2,3-dihydroxypropyl acrylate, 1,1-dimethyl-2-hydroxyethyl acrylate, 2-hydroxybutyl acrylate, 3-hydroxybutyl acrylate 4-hydroxybutyl acrylate, and combinations thereof. An especially preferred hydroxyalkyl (meth)acrylate is 4-hydroxybutyl acrylate.

The reactive monomer mixture of Composition (A) may comprise the hydroxyalkyl (meth)acrylate in an amount between about 0.01 and about 25 weight percent, between about 1 weight percent and 20 weight percent, between about 5 weight percent and about 20 weight percent, or between about 5 weight percent and about 15 weight percent.

In some aspects of the invention, the reactive monomer mixture of Composition (A) further comprises a free radical polymerization initiator. Some initiators thermally decompose into radicals, such as peroxides, peracids, and azo compounds. The rate of initiation depends on the chemical structure of these “thermal initiators” as well as the polymerization temperature. Other initiators generate radicals photochemically, such as aromatic alpha-hydroxy ketones, alkoxyoxybenzoins, acetophenones, monoacylphosphine oxides, bisacylphosphine oxides, and the like. The rate of initiation depends on the chemical structure of these “photo-initiators” as well as the irradiation intensity, irradiation wavelength, the concentration of any inhibitors, and the level of oxygen gas in the system. Commercially available (from IGM Resins B.V., The Netherlands) ultraviolet and/or visible light initiator systems include Omnirad 403, Omnirad 819, Omnirad 1173, Omnirad 1700, and Omnirad 1870. These systems and other photoinitiators which may be used are disclosed in Volume III, Photoinitiators for Free Radical Cationic & Anionic Photopolymerization, 2nd Edition by J. V. Crivello & K. Dietliker; edited by G. Bradley; John Wiley and Sons; New York; 1998.

The reactive monomer mixture of Composition (A) may comprise a thermal initiator, a photo-initiator, or a combination thereof. Preferably, reactive monomer mixture of Composition (A) includes only a thermal initiator or only a photo-initiator. A preferred thermal initiator is azobisisobutyronitrile. Preferred photo-initiators are monoacylphosphine oxides, bisacylphosphine oxides, and combinations thereof. Most preferably, the reactive monomer mixture of Composition (A) includes a bisacylphosphine oxide photo-initiator. An especially preferred photo-initiator is bis(2,4,6-trimethylbenzoyl)-phenylphosphine oxide.

The reactive monomer mixture of Composition (A) may comprise a free radical polymerization initiator in an amount between about 0.01 weight percent and about 5 weight percent, between about 0.1 weight percent and about 3 weight percent, between about 0.1 weight percent and about 2 weight percent, between about 0.1 weight percent and about 1 weight percent, or between about 0.2 weight percent and about 0.6 weight percent. When there is more than one free radical polymerization initiator in the reactive monomer mixture, including thermal initiators, photo-initiators, or combinations thereof, the above concentration ranges apply to the total amount of free radical polymerization initiator regardless of chemical structure or decomposition means.

In some aspects of the invention, the reactive monomer mixture of Composition (A) further comprises at least one UV absorbing compound. Typical UV absorbing compounds are benzotriazoles such as 2-(2′-hydroxy-5-methacrylyloxyethylphenyl)-2H-benzotriazole.

In some aspects of the invention, the reactive monomer mixture of Composition (A) further comprises at least one UV/HEV absorbing compound. Preferably, the UV/HEV absorbing compound is 3-(3-(tert-butyl)-5-(5-chloro-2H-benzo[d][1,2,3]triazol-2-yl)-4-hydroxyphenyl)-propyl methacrylate. More preferably, the UV/HEV absorbing compounds have the chemical structures shown in Formula V and Formula VI:

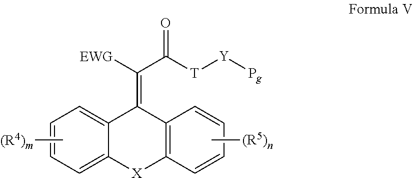

wherein:
m and n are independently 0, 1, 2, 3, or 4;
T is a bond, O, or NR⁶;
X is O, S, NR, SO, or SO₂;
Y is a linking group;
P_{g} is a polymerizable group;
R⁶ at each occurrence is independently H, C₁-C₆ alkyl, cycloalkyl, heterocycloalkyl, aryl, heteroaryl, or Y—P_{g};
R⁴ and R⁵, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR⁷R⁸, or benzyl, wherein R⁷ and R⁸ are independently H or C₁-C₆ alkyl, or two adjacent R⁴ or R⁵ groups, together with the carbon atoms to which they are attached, combine to form a cycloalkyl or aryl ring; and EWG is an electron withdrawing group, preferably cyano;

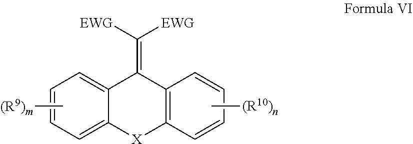

wherein:
m and n are independently 0, 1, 2, 3, or 4;
X is O, S, NR¹¹, SO, or SO₂;
R¹¹ at each occurrence is independently H, C₁-C₆ alkyl, cycloalkyl, heterocycloalkyl, aryl, heteroaryl, or Y—P_{g}, wherein Y is a linking group and P_{g} is a polymerizable group;
R⁹ and R¹⁰, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹²R¹³, or benzyl, wherein R¹² and R¹³ are independently H or C₁-C₆ alkyl, two adjacent R⁹ or R¹⁰ groups, together with the carbon atoms to which they are attached, combine to form a cycloalkyl or aryl ring, Y—P_{g}, or T-Y—P_{g}, wherein T is a bond, O, or NR¹¹; and EWG is an electron withdrawing group, preferably cyano.

UV/IEV absorbing compounds of Formulae V and VI preferably contain one or two Y—P_{g} groups. More preferably, the UV/IEV absorbing compounds contain one Y—P_{g} group.

The reactive monomer mixture of Composition (A) may comprise an UV/HEV absorbing compound having a chemical structure of Formula V, such as 2-(2-cyano-2-(9H-thioxanthen-9-ylidene)acetamido)ethyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 2-(2-cyano-2-(2-methoxy-10-propylacridin-9(10H)-ylidene)acetamido)ethyl methacrylate, 2-(2-cyano-2-(2-methoxy-10-butylacridin-9(10H)-ylidene)acetamido)ethyl methacrylate, or any combination thereof. Preferably, the UV/HEV absorbing compound is 2-(2-cyano-2-(9H-thioxanthen-9-ylidene)acetamido)ethyl methacrylate.

The reactive monomer mixture of Composition (A) may comprise an UV/HEV absorbing compound having a chemical structure of Formula VI, preferably such as 3-((9-(dicyanomethylene)-9H-xanthen-3-yl)oxy)propyl methacrylate.

The reactive monomer mixture of Composition (A) may comprise the UV absorbing compound in an amount between about 0.01 weight percent and about 5 weight percent, between about 0.05 weight percent and about 3 weight percent, between about 0.1 weight percent and about 3 weight percent, between about 0.1 weight percent and about 2 weight percent, between about 0.1 weight percent and about 1 weight percent, or between about 0.1 weight percent and about 0.5 weight percent.

The reactive monomer mixture of Composition (A) may comprise the UV/HEV absorbing compound in an amount between about 0.01 weight percent and about 5 weight percent, between about 0.05 weight percent and about 3 weight percent, between about 0.1 weight percent and about 3 weight percent, between about 0.1 weight percent and about 2 weight percent, between about 0.1 weight percent and about 1 weight percent, or between about 0.1 weight percent and about 0.5 weight percent.

In some aspects of the invention, the reactive monomer mixture of Composition (A) further comprises a hydrophilic component. Preferably, the hydrophilic component is poly(ethylene glycol)-containing monomer or macromer depending on the number of repeating units having the chemical structure shown in Formula VII

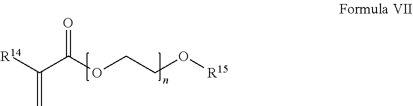

wherein R¹⁴ is a hydrogen atom or methyl and R¹⁵ are selected from the group consisting of hydrogen, C₁-C₆ alkyl, and aryl; and wherein “n” is an integer from 1 to 25, including 1, 2, 3, 4, 5, 6, 7, 8, 9, 10, 11, 12, 13, 14, 15, 16, 17, 18, 19, 20, 21, 22, 23, 24, and 25. Preferably, R¹⁴ is methyl. Preferably, R¹⁵ is a hydrogen atom, methyl, or phenyl. Preferably, “n” is an integer between 1 and 15. More preferably, “n” is an integer between 1 and 8, including 1, 2, 3, 4, 5, 6, 7, and 8.

More preferably, the poly(ethylene glycol)-containing macromer is selected from the group consisting of poly(ethylene glycol) (meth)acrylate, poly(ethylene glycol) methyl ether (meth)acrylate, poly(ethylene glycol) phenyl ether (meth)acrylate, and combinations thereof. Most preferably, the poly(ethylene glycol)-containing macromer is selected from the group consisting of poly(ethylene glycol) methacrylate, poly(ethylene glycol) phenyl ether acrylate, and combinations thereof.

In some embodiments, the poly(ethylene glycol)-containing monomer has a number-average molecular weight (Mₙ) of about 200 g/mol to about 1000 g/mol, including 200 g/mol, 220 g/mol, 240 g/mol, 260 g/mol, 280 g/mol, 300 g/mol, 320 g/mol, 340 g/mol, 360 g/mol, 380 g/mol, 400 g/mol, 420 g/mol, 440 g/mol, 460 g/mol, 480 g/mol, 500 g/mol, 520 g/mol, 540 g/mol, 560 g/mol, 580 g/mol, 600 g/mol, 620 g/mol, 640 g/mol, 660 g/mol, 680 g/mol, 700 g/mol, 720 g/mol, 740 g/mol, 760 g/mol, 780 g/mol, 800 g/mol, 820 g/mol, 840 g/mol, 860 g/mol, 880 g/mol, 900 g/mol, 920 g/mol, 940 g/mol, 960 g/mol, 980 g/mol, and 1000 g/mol. In some embodiments, the poly(ethylene glycol)-containing monomer has a number-average molecular weight (Mₙ) of about 200 g/mol to about 400 g/mol, including 200 g/mol, 220 g/mol, 240 g/mol, 260 g/mol, 280 g/mol, 300 g/mol, 320 g/mol, 340 g/mol, 360 g/mol, 380 g/mol, and 400 g/mol.

In some aspects of the invention, the reactive monomer mixture of Composition (A) further comprises a cycloalkyl(alkyl) (meth)acrylate. Preferred cycloalkyl(alkyl) (meth)acrylates are cyclohexylmethyl acrylate, 2-cyclohexylethyl acrylate, and 3-cyclohexylpropyl acrylate.

In some aspects of the invention, the reactive monomer mixture of Composition (A) further comprises at least one diluent. Any organic solvent may be used to dissolve the components of reactive monomer mixture. Preferably, the organic solvent is also chosen to be extractable from Composition (A) after the free radical polymerization is completed and exhibit a boiling point sufficiently higher than the polymerization temperature to avoid the formation of bubbles and cavitation.

In some aspects of the invention, Composition (A) exhibits the following combinations of refractive index and Abbe number: (a) a refractive index of at least 1.45 and an Abbe number of at least 45; (b) a refractive index of at least 1.48 and an Abbe number of at least 48; (c) a refractive index of at least 1.49 and an Abbe number of at least 49; (d) a refractive index of at least 1.50 and an Abbe number of at least 50; (e) a refractive index of at least 1.51 and an Abbe number of at least 51; or (f) a refractive index of at least 1.52 and an Abbe number of at least 52.

In some aspects of the invention, Composition (A) exhibits a water content between about 0.01 weight percent and about 15 weight percent; between about 0.1 weight percent and about 10 weight percent; between about 0.5 weight percent and about 5 weight percent; between about 0.5 weight percent and about 3 weight percent; or between about 1 weight percent and about 2 weight percent. A preferred water content is between about 1 weight percent and about 2 weight percent.

In some aspects of the invention, Composition (A) exhibits a storage modulus between about 1 megapascal and about 100 megapascals; between about 10 megapascal and about 90 megapascals; between about 20 megapascal and about 80 megapascals; between about 30 megapascal and about 80 megapascals; or between about 40 megapascal and about 80 megapascals. A preferred storage modulus is between about 40 megapascal and about 80 megapascals.

In a specific aspect of the invention, the reactive monomer mixture comprises: 2-((butylcarbamoyl)oxy)ethyl acrylate at 24-28 weight percent; 4-hydroxybutyl acrylate at 10 weight percent; tricyclo[5.2.1.0^{2,6}]decanedimethanol diacrylate at 1.5 weight percent; 3-((9-(dicyanomethylene)-9H-xanthen-3-yl)oxy)propyl methacrylate at 0.2 weight percent; bis(2,4,6-trimethylbenzoyl)-phenylphosphineoxide at 0.45 weight percent; ethylene glycol dicyclopentenyl ether acrylate at 55-59 weight percent; and n-hexyl acrylate at 3-6 weight percent; wherein the concentration of ethylene glycol dicyclopentenyl ether acrylate and n-hexyl acrylate vary, but the components of the reactive monomer mixture add up to 100 weight percent; wherein the composition exhibits a refractive index of at least 1.50 and an Abbe number of at least 50; and wherein the storage modulus is between 1 megapascal and 100 megapascals [“Composition (B)” ].

In another specific aspect of the invention, the reactive monomer mixture comprises: 2-oxo-2-(decylamino)ethyl (meth)acrylate at 16-18 weight percent; 4-hydroxybutyl acrylate at 10 weight percent; tricyclo[5.2.1.0^{2,6}]decanedimethanol diacrylate at 1.5 weight percent; 3-((9-(dicyanomethylene)-9H-xanthen-3-yl)oxy)propyl methacrylate at 0.2 weight percent; bis(2,4,6-trimethylbenzoyl)-phenylphosphineoxide at 0.45 weight percent; ethylene glycol dicyclopentenyl ether acrylate at 65-67 weight percent; and n-hexyl acrylate at 5-7 weight percent; wherein the concentration of 2-oxo-2-(decylamino)ethyl (meth)acrylate, ethylene glycol dicyclopentenyl ether acrylate, and n-hexyl acrylate vary, but the components of the reactive monomer mixture add up to 100 weight percent; wherein the composition exhibits a refractive index of at least 1.50 and an Abbe number of at least 50; and wherein the storage modulus is between 1 megapascal and 100 megapascals [“Composition (C)” ].

Another aspect of the invention is a compound [“Compound (A)” ] having the chemical structure depicted by Formula II, P_{g}-L-CONRTR², wherein P_{g} is a polymerizable group, L is a linking group, and R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups. Compound (A) is a pendant amide monomer.

Non-limiting examples of the polymerizable group of Compound (A) include (meth)acrylates, (meth)acrylamides, N-vinyllactams, N-vinylamides, styrenes, vinyl ethers, O-vinylcarbamates, O-vinylcarbonates, and other vinyl groups. Preferably, the polymerizable groups comprise (meth)acrylate, (meth)acrylamide, N-vinyllactam, N-vinylamide groups, and mixtures thereof. More preferably, the polymerizable groups comprise (meth)acrylates, (meth)acrylamides, and combinations thereof. Most preferably, the polymerizable groups comprise (meth)acrylates.

Non-limiting examples of the linking group of Compound (A) include alkylene, haloalkylene, amide, amine, alkyleneamine, carbamate, ester, arylene, heteroarylene, cycloalkylene, heterocycloalkylene, alkyleneoxy, oxaalkylene, thiaalkylene, and haloalkyleneoxy. Preferably, the linking groups comprise ester, amide, C₁-C₈ alkylene, C₁-C₈ oxaalkylene, C₁-C₈ alkylene-ester-C₁-C₈ alkylene, C₁-C₈ alkylene-amide-C₁-C₈ alkylene. More preferably, the linking groups comprise C₁-C₈ alkylene and C₁-C₅ oxaalkylene. Most preferably, the linking groups comprise C₁-C₈ alkylene. Especially preferred linking groups are unsubstituted C₁-C₄ alkylene.

In some aspects of the invention, Compound (A) having the combination of (meth)acrylate polymerization groups and unsubstituted alkylene linking groups are preferred. In particular, Compound (A) having the chemical structure depicted in Formula IV is most preferred:

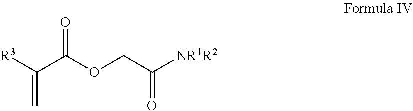

wherein R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups, and R³ is H or methyl.

Non-limiting examples of Compound (A) having the chemical structure of Formula IV include 2-oxo-2-(methylamino)ethyl (meth)acrylate, 2-oxo-2-(ethylamino)ethyl (meth)acrylate, 2-oxo-2-(propylamino)ethyl (meth)acrylate, 2-oxo-2-(butylamino)ethyl (meth)acrylate, 2-oxo-2-(pentylamino)ethyl (meth)acrylate, 2-oxo-2-(hexylamino)ethyl (meth)acrylate, 2-oxo-2-(heptylamino)ethyl (meth)acrylate, 2-oxo-2-(octylamino)ethyl (meth)acrylate, 2-oxo-2-(nonylamino)ethyl (meth)acrylate, 2-oxo-2-(decylamino)ethyl (meth)acrylate, 2-oxo-2-(undecylamino)ethyl (meth)acrylate, 2-oxo-2-(dodecylamino)ethyl (meth)acrylate, 2-oxo-2-(tridecylamino)ethyl (meth)acrylate, 2-oxo-2-(tetradecylamino)ethyl (meth)acrylate, 2-oxo-2-((3-methoxypropyl)amino)ethyl (meth)acrylate, 2-oxo-2-((3-ethoxypropyl)amino)ethyl (meth)acrylate, 2-oxo-2-((cyclohexylmethyl)amino)ethyl (meth)acrylate, 2-oxo-2-(benzylamino)ethyl (meth)acrylate, 2-oxo-2-(phenethylamino)ethyl (meth)acrylate, 2-oxo-2-((thiophen-2-ylmethyl)amino)ethyl (meth)acrylate, 2-oxo-2-((2,3-dihydroxypropyl)amino)ethyl acrylate, and 2-(dimethylamino)-2-oxoethyl methacrylate.

In some aspects of the invention, the presently disclosed subject matter provides a composition made by free radical polymerization of a reactive monomer mixture comprising:
(a) a pendant amide monomer having the chemical structure of Formula IV

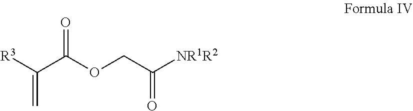

wherein R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups and R³ is H or methyl [“Composition (D)”].

Non-limiting examples of the pendant amide monomer having the chemical structure of Formula IV include 2-oxo-2-(methylamino)ethyl (meth)acrylate, 2-oxo-2-(ethylamino)ethyl (meth)acrylate, 2-oxo-2-(propylamino)ethyl (meth)acrylate, 2-oxo-2-(butylamino)ethyl (meth)acrylate, 2-oxo-2-(pentylamino)ethyl (meth)acrylate, 2-oxo-2-(hexylamino)ethyl (meth)acrylate, 2-oxo-2-(heptylamino)ethyl (meth)acrylate, 2-oxo-2-(octylamino)ethyl (meth)acrylate, 2-oxo-2-(nonylamino)ethyl (meth)acrylate, 2-oxo-2-(decylamino)ethyl (meth)acrylate, 2-oxo-2-(undecylamino)ethyl (meth)acrylate, 2-oxo-2-(dodecylamino)ethyl (meth)acrylate, 2-oxo-2-(tridecylamino)ethyl (meth)acrylate, 2-oxo-2-(tetradecylamino)ethyl (meth)acrylate, 2-oxo-2-((3-methoxypropyl)amino)ethyl (meth)acrylate, 2-oxo-2-((3-ethoxypropyl)amino)ethyl (meth)acrylate, 2-oxo-2-((cyclohexylmethyl)amino)ethyl (meth)acrylate, 2-oxo-2-(benzylamino)ethyl (meth)acrylate, 2-oxo-2-(phenethylamino)ethyl (meth)acrylate, 2-oxo-2-((thiophen-2-ylmethyl)amino)ethyl (meth)acrylate, 2-oxo-2-((2,3-dihydroxypropyl)amino)ethyl acrylate, and 2-(dimethylamino)-2-oxoethyl methacrylate.

In some aspects of the invention, the reactive monomer mixture of Composition (D) further includes additional components selected from the group consisting of hydrophilic components, silicone-containing components, alkyl (meth)acrylates, hydroxyalkyl (meth)acrylates, cycloalkyl(alkyl) (meth)acrylates, polyamides, UV absorbing compounds, UV/HEV absorbing compounds, visibility tints, cross-linking agents, free radial polymerization initiators, and diluents. These additional components are either defined herein or as broadly disclosed in U.S. Pat. No. 10,935,695 which is incorporated herein by reference in its entirety.

### C. Ophthalmic Devices

In some aspects of the invention, the presently disclosed subject matter provides a device comprising the compositions as described immediately hereinabove, namely Composition (A), Composition (B), Composition (C), and Composition (D).

In other aspects, the device comprises an ophthalmic lens, inlay, outlay, implant, or insert selected from an intraocular implant, intraocular lens, phakic intraocular lens, a contact lens, an orthokeratology lens, a rigid gas permeable lens, a corneal inlay, a corneal outlay, and a corneal insert. In yet other aspects of the invention, the above ophthalmic devices may be coated after fabrication to modify the surface properties of the lenses or implants. Any coating methodology may be used including but not limited to dip coating, spray coating, spin coating, chemical vapor deposition, sALD, plasma treatment, and the like. The coating methodologies may also include a curing step by any known chemistry, such a photochemical polymerization, to create a robust coating.

In specific aspects, the ophthalmic device is an intraocular lens or implant. More specifically, the presently disclosed subject matter provides intraocular implants and/or lenses made at least partially or completely from the compositions described herein. Such intraocular implants or lenses can include an optic portion and one or more haptic portions. Typically, the compositions of the presently disclosed subject matter will make up part or all of the optic portion of the intraocular implant or lens. In some aspects, the optic portion of the implant or lens will have a core made from one of the compositions described herein surrounded by different polymer or material. Implants or lenses in which the optic portion is made up of at least partially of one of the compositions of the presently disclosed subject matter will usually also have a haptic portion. The haptic portion can also be made of polymer of the disclosure or can be made of a different material, for example another polymer.

In yet other aspects, the intraocular implant or lens of the presently disclosed subject matter is a one-piece lens having a soft, foldable central optic region and an outer peripheral region (haptic-region) in which both regions are made of the same polymer. In other embodiments, the optic and haptic regions can be formed from different types of polymers or materials, if desired. Some implants or lenses can also have haptic portions that are made up of different materials, for example where one or more haptic portions is made from the same material as the optic portion and other haptic portions are made of materials other than a polymer of the disclosure. Multicomponent implants or lenses can be made by embedding one material in the other, concurrent extrusion processes, solidifying the hard material about the soft material, or forming an interpenetrating network of the rigid component into a preformed hydrophobic core. In instances where one or more haptic portions are made from a different material than the optic portion of the lens, the haptic portion can be attached to the optic portion in any manner known in the art, such as by drilling a hole or holes in the optic portion and inserting the haptic portion.

The compositions described herein have been designed so that they are capable of being folded so that the intraocular lens can be inserted into the eye of an individual through a small incision. In some instances that incision will be less than 2.5 millimeters in diameter; in some instances that incision will be less than 2 millimeters in diameter. The haptic portion of the lens provides the required support for the implant or lens in the eye after insertion and unfolding of the lens and tends to help stabilize the position of the lens after insertion and the closure of the incision. The shape of the haptic portion design is not particularly limited and can be any desired configuration, for example, either a plate type or graduated thickness spiral filaments, also known as a C-loop design.

The optic portion of the intraocular lens can be approximately 2-6 millimeters in diameter prior to hydration. The 2-6 millimeter diameter is fairly standard in the art and is generally chosen to cover the pupil in its fully dilated state under naturally occurring conditions. However, other sizes are contemplated and the presently disclosed subject matter is not limited to any particular diameter or size of intraocular lens. Furthermore, it is not necessary that the lens optic portion be circular; it could also be oval, square, or any other shape as desired.

The intraocular lens can further include one or more non-optical haptic components extending away from the outermost peripheral surface of the optic portion. The haptic components can be of any desired shape, for example, graduated spiral filaments or flat plate sections and are used to support the lens within the posterior chamber of the eye. Lenses having any desired design configuration can be fabricated. Should the intraocular lens include other components besides the optical and haptic portions, such other portions can be made of a polymer as are the haptic and optic portions, or if desired, another material.

The intraocular implants lenses may be inserted into the eye in any manner known in the art. For example, the intraocular lens may be folded prior to insertion into the eye using an intraocular lens inserter or by small, thin forceps of the type typically used by ophthalmic surgeons. After the implant or lens is in the targeted location, it is released to unfold. As is well known in the art, typically the lens that is to be replaced is removed prior to insertion of the intraocular lens. The intraocular lens of the presently disclosed subject matter can be made of a generally physiologically inert soft polymeric material that is capable of providing a clear, transparent, refractive lens body even after folding and unfolding. In some embodiments, the foldable intraocular lens of the presently disclosed subject matter can be inserted into any eye by injection whereby the mechanically compliant material is folded and forced through a small tube such as a 1-millimeter to 3-millimeter inner diameter tube.

There are other aspects of the invention in which monomers having the chemical structure of Formula IV are used to make contact lenses, including but not limited to, soft hydrogel contact lenses, soft silicone hydrogel contact lenses, hard contact lenses, rigid gas permeable lenses, and orthokeratological lenses. Further aspects of the invention include the making of reactive monomer mixture components from monomers having the chemical structure of Formula IV including but not limited to internal wetting agents and plasticizers. Other aspects of the invention include the making of packing solution additives from monomers having the chemical structure of Formula IV such as polymeric comfort agents and the like.

### D. Methods for Making Ophthalmic Devices

In still yet other embodiments, the presently disclosed subject matter provides a method for making an ophthalmic device, the method comprising: (a) providing any of the compositions described herein [e.g., Composition (A), Composition (B), Composition (C), or Composition (D)] and (b) forming an ophthalmic device. In other embodiments, the presently disclosed subject matter provides a method for making an ophthalmic device, the method comprising: (a) preparing a sample from any of the compositions described herein [e.g., Composition (A), Composition (B), Composition (C), or Composition (D)] and (b) machining an ophthalmic device from the sample. The sample may be of any shape or size, but typically has a circular or rectangular cross-section. A preferred sample is a round disk. In still other embodiments, the presently disclosed subject matter provides a method for making an ophthalmic device, the method comprising molding an ophthalmic device from any of the compositions described herein [e.g., Composition (A), Composition (B), Composition (C), or Composition (D)]. Alternatively, the method comprises (a) providing any of the compositions described herein [e.g., Composition (A), Composition (B), Composition (C), or Composition (D)] in a mold assembly, (b) forming an ophthalmic device by a photopolymerization reaction, and (c) demolding the ophthalmic device from the mold assembly. Alternatively, the method comprises (a) providing any of the compositions described herein [e.g., Composition (A), Composition (B), Composition (C), or Composition (D)] in a mold assembly, (b) forming an ophthalmic device by a thermal polymerization reaction, and (c) demolding the ophthalmic device from the mold assembly. In still other embodiments, the presently disclosed subject matter provides a method for making an ophthalmic device, the method comprising molding an ophthalmic device from any of the compositions described herein [e.g., Composition (A), Composition (B), Composition (C), or Composition (D)], and then refining the surface via lathing. In certain embodiments of the above methods, the method further comprises the step of extracting the ophthalmic device with a solvent. In certain embodiments, the method further comprises the step of hydrating the extracted ophthalmic device with at least one aqueous solution. In particular embodiments, the method further comprises an irradiation step using a laser, which in certain embodiments, is a two-photon laser, which in more certain embodiments, is a femtosecond two photon laser. In more particular embodiments, the method further comprises a step of sterilizing the ophthalmic device. The ophthalmic device may be sterilized by known means such as, but not limited to, autoclaving and exposure of ethylene oxide gas.

The present invention provides a method for making an ophthalmic device, the method comprising the steps of (a) providing a composition comprised of a compatibilizing monomer, a cross-linking agent and a an ethylene glycol dicyclopentenyl ether (meth)acrylate and (b) forming an ophthalmic device; alternatively, (a) molding the device from a composition comprised of a compatibilizing monomer, a cross-linking agent and a an ethylene glycol dicyclopentenyl ether (meth)acrylate; alternatively, (a) providing a composition comprised of a compatibilizing monomer, a cross-linking agent and a an ethylene glycol dicyclopentenyl ether (meth)acrylate in a mold assembly, (b) forming an ophthalmic device, and (c) demolding the ophthalmic device from the mold assembly; and alternatively, (a) providing a composition comprised of a compatibilizing monomer, a cross-linking agent and a an ethylene glycol dicyclopentenyl ether (meth)acrylate in a mold assembly, (b) forming an ophthalmic device by a photopolymerization reaction, and (c) demolding the ophthalmic device from the mold assembly. In certain aspects of any of the aforementioned methods of making an ophthalmic device, the method further comprises a step of extracting the ophthalmic device with a solvent. In certain other aspects of any of the aforementioned methods of making an ophthalmic device, the method further comprises a step of hydrating the extracted ophthalmic device with at least one aqueous solution. In yet other aspects of any of the aforementioned methods of making an ophthalmic device, the method further comprises a step of sterilizing the ophthalmic device. The ophthalmic device may be sterilized by known means such as, but not limited to, autoclaving and exposure to ethylene oxide gas. In certain embodiments of any of the aforementioned methods of making an ophthalmic device, the method further comprises an irradiation step using a laser either before or after sterilization, including after the ophthalmic device has been implanted in a human. The laser may be a two-photon laser, such as but not limited to, a femtosecond two photon laser. In certain embodiments of any of the aforementioned methods of making an ophthalmic device, the method further comprises a coating step in which the formed device is coated using any coating methodology including but not limited to dip coating, spray coating, spin coating, chemical vapor deposition, plasma treatment, sALD, and the like. The coating methodologies may also include a curing step by any known chemistry, such a photochemical polymerization, to create a robust coating.

Another specific aspect of the invention related to any methods of making ophthalmic devices from the compositions disclosed herein, including Composition (A), Composition (B), Composition (C), and Composition (D), is a forming or molding step comprising a photopolymerization reaction involving irradiating the mold assembly from the top and the bottom with 435 nanometer light emitting diodes having the following intensity profile: 20 minutes at 5 mW/cm² (2.5 mW/cm² top and 2.5 mW/cm² bottom), 20 minutes at 10 mW/cm² (5 mW/cm² top and 5 mW/cm² bottom), 20 minutes at 20 mW/cm² (10 mW/cm² top and 10 mW/cm² bottom); and 30 minutes at 30 mW/cm² (15 mW/cm² top and 15 mW/cm² bottom).

Yet another specific aspect of the invention related to any methods of making ophthalmic devices from the compositions disclosed herein, including Composition (A), Composition (B), Composition (C), and Composition (D), is a solvent extraction step involving solvents selected from the group consisting of acetonitrile, isopropanol, and aqueous solutions of acetonitrile or isopropanol.

In another embodiment of the invention, compounds having the chemical structure of Formula IV can be made by a method comprising (a) reacting a primary or secondary amine with methyl glycolate to form a N-alkyl-2-hydroxyacetamide or N-alkyl (R′)—N-alkyl (R″)-2-hydroxyacetamide and (b) reacting the N-alkyl-2-hydroxyacetamide or N-alkyl (R′)—N-alkyl (R″)-2-hydroxyacetamide with (meth)acryloyl chloride.

### CLAUSES

Certain aspects of the invention as described hereto can be combined in whole or in part. The following clauses list some non-limiting embodiments of the disclosure.
Clause 1. A composition made by free radical polymerization of a reactive monomer mixture comprising: a compatibilizing monomer selected from the group consisting of a pendant carbamate monomer having a chemical structure of Formula I, a pendant amide monomer having a chemical structure of Formula II, and combinations thereof:

P_{g}-L-OCONR¹R² Formula I

P_{g}-L-CONR¹R² Formula II,

wherein P_{g} is a polymerizable group, L is a linking group, and R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl and heteroarylalkyl groups; a cross-linking agent; and an ethylene glycol dicyclopentenyl ether (meth)acrylate; wherein the concentration of the ethylene glycol dicyclopentenyl ether (meth)acrylate in the reactive monomer mixture excluding any diluent is greater than or equal to 20 weight percent; and wherein the composition exhibits a refractive index of at least 1.45 and an Abbe number of at least 39.
Clause 2. The composition of clause 1, wherein the polymerizable group of the compatibilizing monomer is a (meth)acrylate and the linking group of the compatibilizing monomer is an unsubstituted alkylene group.
Clause 3. The composition of clause 2, wherein the compatibilizing monomer is selected from the group consisting of Formula III, Formula IV, and combinations thereof:

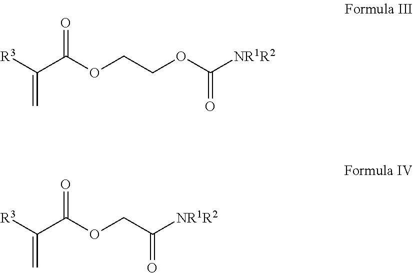

wherein R³ is H or methyl.
Clause 4. The composition of clause 3, wherein the compatibilizing monomer is selected from the group consisting of: 2-((methylcarbamoyl)oxy)ethyl (meth)acrylate, 2-((ethylcarbamoyl)oxy)ethyl (meth)acrylate, 2-((propylcarbamoyl)oxy)ethyl (meth)acrylate, 2-((butylcarbamoyl)oxy)ethyl (meth)acrylate, 2-((pentylcarbamoyl)oxy)ethyl (meth)acrylate,
2-((hexylcarbamoyl)oxy)ethyl (meth)acrylate, 2-((heptylcarbamoyl)oxy)ethyl (meth)acrylate, 2-((octylcarbamoyl)oxy)ethyl (meth)acrylate, 2-((nonylcarbamoyl)oxy)ethyl (meth)acrylate, 2-((decylcarbamoyl)oxy)ethyl (meth)acrylate, 2-((undecylcarbamoyl)oxy)ethyl (meth)acrylate, 2-((dodecylcarbamoyl)oxy)ethyl (meth)acrylate, 2-((tridecylcarbamoyl)oxy)ethyl (meth)acrylate, 2-((tetradecylcarbamoyl)oxy)ethyl (meth)acrylate, 2-((pentadecylcarbamoyl)oxy)ethyl (meth)acrylate, 2-((hexadecylcarbamoyl)oxy)ethyl (meth)acrylate, 2-((heptadecylcarbamoyl)oxy)ethyl (meth)acrylate, 2-oxo-2-(methylamino)ethyl (meth)acrylate, 2-oxo-2-(ethylamino)ethyl (meth)acrylate, 2-oxo-2-(propylamino)ethyl (meth)acrylate, 2-oxo-2-(butylamino)ethyl (meth)acrylate, 2-oxo-2-(pentylamino)ethyl (meth)acrylate, 2-oxo-2-(hexylamino)ethyl (meth)acrylate, 2-oxo-2-(heptylamino)ethyl (meth)acrylate, 2-oxo-2-(octylamino)ethyl (meth)acrylate, 2-oxo-2-(nonylamino)ethyl (meth)acrylate, 2-oxo-2-(decylamino)ethyl (meth)acrylate, 2-oxo-2-(undecylamino)ethyl (meth)acrylate, 2-oxo-2-(dodecylamino)ethyl (meth)acrylate, 2-oxo-2-(tridecylamino)ethyl (meth)acrylate, 2-oxo-2-(tetradecylamino)ethyl (meth)acrylate, 2-oxo-2-((3-methoxypropyl)amino)ethyl (meth)acrylate, 2-oxo-2-((3-ethoxypropyl)amino)ethyl (meth)acrylate, 2-oxo-2-((cyclohexylmethyl)amino)ethyl (meth)acrylate, 2-oxo-2-(benzylamino)ethyl (meth)acrylate, 2-oxo-2-(phenethylamino)ethyl (meth)acrylate, 2-oxo-2-((thiophen-2-ylmethyl)amino)ethyl (meth)acrylate, 2-oxo-2-((2,3-dihydroxypropyl)amino)ethyl (meth)acrylate, 2-(dimethylamino)-2-oxoethyl methacrylate, and combinations thereof.
Clause 5. The composition of clause 4, wherein the compatibilizing monomer is selected from the group consisting of 2-((butylcarbamoyl)oxy)ethyl (meth)acrylate, 2-oxo-2-(pentylamino)ethyl (meth)acrylate, 2-oxo-2-(octylamino)ethyl (meth)acrylate, 2-oxo-2-(decylamino)ethyl (meth)acrylate, and combinations thereof.
Clause 6. The composition of clause 5, wherein the compatibilizing monomer is selected from the group consisting of 2-((butylcarbamoyl)oxy)ethyl acrylate, 2-oxo-2-(pentylamino)ethyl acrylate, and combinations thereof.
Clause 7. The composition of any of clauses 1-6, wherein the crosslinking agent is selected from the group consisting of tricyclo[5.2.1.0^{2,6}]decanedimethanol di(meth)acrylate, ethylene glycol di(meth)acrylate, diethylene glycol di(meth)acrylate, triethylene glycol di(meth)acrylate, tetraethylene glycol di(meth)acrylate, trimethylolpropane tri(meth)acrylate, 1,4-butanediol di(meth)acrylate, 1,5-pentanediol di(meth)acrylate, 1,6-hexanediol di(meth)acrylate, 1,7-heptanediol di(meth)acrylate, 1,8-octanediol di(meth)acrylate, 1,9-nonanediol di(meth)acrylate, 1,10-decanediol di(meth)acrylate, 1,11-undecanediol di(meth)acrylate, 1,12-dodecanediol di(meth)acrylate, 1,13-tridecanediol di(meth)acrylate, 1,14-tetradecanediol di(meth)acrylate, 1,15-pentadecanediol di(meth)acrylate, 1,16-hexadecanediol di(meth)acrylate, 1,17-heptadecanediol di(meth)acrylate, 1,18-octadecanediol di(meth)acrylate, glycerol tri(meth)acrylate, triallyl cyanurate, methylene bis(meth)acrylamide, poly(ethylene glycol) di(meth)acrylate, and any combination thereof.
Clause 8. The composition of clause 7, wherein the crosslinking agent is selected from the group consisting of tricyclo[5.2.1.0^{2,6}]decanedimethanol diacrylate, ethylene glycol dimethacrylate, and combinations thereof.
Clause 9. The composition of clause 8, wherein the crosslinking agent is tricyclo[5.2.1.0^{2,6}]decanedimethanol diacrylate.
Clause 10. The composition of any of clauses 1-9, wherein the ethylene glycol dicyclopentenyl ether (meth)acrylate is ethylene glycol dicyclopentenyl ether acrylate.
Clause 11. The composition of any of clauses 1-10, further comprising an aliphatic alkyl (meth)acrylate monomer, wherein the alkyl group contains between one and twenty carbon atoms.
Clause 12. The composition of clause 11, wherein the aliphatic alkyl (meth)acrylate is n-hexyl acrylate.
Clause 13. The composition of clause 12, wherein the reactive monomer mixture comprises n-hexyl acrylate in an amount between about 0.01 and about 20 weight percent, between about 1 weight percent and 20 weight percent, between about 1 weight percent and about 15 weight percent, or between about 1 weight percent and about 10 weight percent.
Clause 14. The composition of any of clauses 1-13, further comprising a hydroxyalkyl (meth)acrylate monomer, wherein the hydroxyalkyl group contains between one and twenty carbon atoms.
Clause 15. The composition of clause 14, wherein the hydroxyalkyl (meth)acrylate monomer is 4-hydroxybutyl acrylate.
Clause 16. The composition of clause 15, wherein the reactive monomer mixture comprises 4-hydroxybutyl acrylate in an amount between about 0.01 and about 25 weight percent, between about 1 weight percent and 20 weight percent, between about 5 weight percent and about 20 weight percent, or between about 5 weight percent and about 15 weight percent.
Clause 17. The composition of any of clauses 1-16, further comprising a free radical polymerization initiator.
Clause 18. The composition of clause 17, wherein the free radical polymerization initiator is a photo-initiator.
Clause 19. The composition of clause 18, wherein the photo-initiator is a bisacylphosphine oxide initiator.
Clause 20. The composition of clause 19, wherein the bisacylphosphine oxide initiator is bis(2,4,6-trimethylbenzoyl)-phenylphosphine oxide.
Clause 21. The composition of any of clauses 17-20, wherein the reactive monomer mixture comprises the free radical polymerization initiator in an amount between about 0.01 weight percent and about 5 weight percent, between about 0.1 weight percent and about 3 weight percent, between about 0.1 weight percent and about 2 weight percent, between about 0.1 weight percent and about 1 weight percent, or between about 0.2 weight percent and about 0.6 weight percent.
Clause 22. The composition of any one of clauses 1-21, wherein the reactive monomer mixture further comprises at least one UV absorbing compound.
Clause 23. The composition of any one of clauses 1-22, wherein the reactive monomer mixture further comprises at least one UV/HEV absorbing compound.
Clause 24. The composition of clause 23, wherein the UV/HEV absorbing compound is selected from the group consisting of 2-(2′-hydroxy-5-methacrylyloxyethylphenyl)-2H-benzotriazole, 2-(2-cyano-2-(9H-thioxanthen-9-ylidene)acetamido)ethyl methacrylate, 3-((9-(dicyanomethylene)-9H-xanthen-3-yl)oxy)propyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 3-(3-(tert-butyl)-5-(5-chloro-2H-benzo[d][1,2,3]triazol-2-yl)-4-hydroxyphenyl)propyl methacrylate, or any combination thereof.
Clause 25. The composition of clause 24, wherein the UV/HEV absorbing compound is 3-((9-(dicyanomethylene)-9H-xanthen-3-yl)oxy)propyl methacrylate.
Clause 26. The composition of any of clauses 23-25, wherein the reactive monomer mixture comprises the UV/HEV absorbing compound in an amount between about 0.01 weight percent and about 5 weight percent, between about 0.05 weight percent and about 3 weight percent, between about 0.1 weight percent and about 3 weight percent, between about 0.1 weight percent and about 2 weight percent, between about 0.1 weight percent and about 1 weight percent, or between about 0.1 weight percent and about 0.5 weight percent.
Clause 27. The composition of any of clauses 1-26, further comprising a hydrophilic component selected from the group consisting of poly(ethylene glycol) (meth)acrylate, poly(ethylene glycol) methyl ether (meth)acrylate, poly(ethylene glycol) phenyl ether (meth)acrylate, and combinations thereof.
Clause 28. The composition of clause 27, wherein the hydrophilic component is selected from the group consisting of poly(ethylene glycol) methacrylate, poly(ethylene glycol) phenyl ether acrylate, and combinations thereof.
Clause 29. The composition of any of clauses 1-28, wherein the reactive monomer mixture comprises the compatibilizing monomer in an amount between about 0.01 weight percent and about 55 weight percent, between about 1 weight percent and about 40 weight percent, between about 5 weight percent and about 35 weight percent, between about 10 weight percent and about 30 weight percent, or between about 20 weight percent and about 30 weight percent.
Clause 30. The composition of any of clauses 1-29, wherein the reactive monomer mixture comprises the crosslinking agent in an amount between about 0.1 weight percent and about 10 weight percent; between about 0.1 weight percent and about 5 weight percent; between about 0.5 weight percent and about 3 weight percent; or between about 1 weight percent and 3 weight percent.
Clause 31. The composition of any of clauses 1-30, wherein the reactive monomer mixture comprises the ethylene glycol dicyclopentenyl ether (meth)acrylate in an amount between about 25 weight percent and about 95 weight percent, between about 30 weight percent and about 75 weight percent, between about 40 weight percent and about 65 weight percent, or between about 45 weight percent and about 60 weight percent.
Clause 32. The composition of any one of clauses 1-31, wherein the reactive monomer mixture further comprises at least one diluent.
Clause 33. The composition of any one of clauses 1-32, wherein the composition has a refractive index of at least 1.45 and an Abbe number of at least 45; wherein the composition has a refractive index of at least 1.48 and an Abbe number of at least 48; wherein the composition has a refractive index of at least 1.49 and an Abbe number of at least 49; wherein the composition has a refractive index of at least 1.50 and an Abbe number of at least 50; wherein the composition has a refractive index of at least 1.51 and an Abbe number of at least 51; or wherein the composition has a refractive index of at least 1.52 and an Abbe number of at least 52.
Clause 34. The composition of any one of clauses 1-33, wherein the composition exhibits a water content between about 0.01 weight percent and about 15 weight percent; between about 0.1 weight percent and about 10 weight percent; between about 0.5 weight percent and about 5 weight percent; between about 0.5 weight percent and about 3 weight percent; or between about 1 weight percent and about 2 weight percent.
Clause 35. The composition of any one of clauses 1-34, wherein the composition exhibits a storage modulus between about 1 megapascal and about 100 megapascals; between about 10 megapascal and about 90 megapascals; between about 20 megapascal and about 80 megapascals; between about 30 megapascal and about 80 megapascals; or between about 40 megapascal and about 80 megapascals.
Clause 36. An ophthalmic device comprising the composition of any one of clauses 1-35.
Clause 37. The ophthalmic device of clause 36 wherein the ophthalmic device comprises an intraocular lens, phakic intraocular lens, contact lens, corneal inlay, corneal outlay, or corneal insert.
Clause 38. The ophthalmic device of clause 37 wherein the ophthalmic device is an intraocular lens.
Clause 39. The ophthalmic device of clause 38 wherein the intraocular lens is coated.
Clause 40. A method for making an ophthalmic device, the method comprising: providing a composition of any one of clauses 1-35; and forming an ophthalmic device.
Clause 41. A method for making an ophthalmic device, the method comprising: preparing a blank from the composition any of clauses 1-35; and machining an ophthalmic device from the blank.
Clause 42. A method for making an ophthalmic device, the method comprising: molding the device from the composition any of clauses 1-35.
Clause 43. A method for making an ophthalmic device, the method comprising: providing a composition of any of clauses 1-35 in a mold assembly; forming an ophthalmic device; and demolding the ophthalmic device from the mold assembly.
Clause 44. A method for making an ophthalmic device, the method comprising: providing a composition of any of clauses 1-35 in a mold assembly; forming an ophthalmic device by a photopolymerization reaction; and demolding the ophthalmic device from the mold assembly.
Clause 45. The method of clause 44, wherein the photopolymerization reaction comprises irradiating the mold assembly from the top and the bottom with 435 nanometer light emitting diodes having an intensity profile: 20 minutes at 5 mW/cm² (2.5 mW/cm² top and 2.5 mW/cm² bottom), 20 minutes at 10 mW/cm² (5 mW/cm² top and 5 mW/cm² bottom), 20 minutes at 20 mW/cm² (10 mW/cm² top and 10 mW/cm² bottom); and 30 minutes at 30 mW/cm² (15 mW/cm² top and 15 mW/cm² bottom).
Clause 46. The method of any of clauses 39-45, further comprising the step of extracting the ophthalmic device with a solvent.
Clause 47. The method of clause 46, wherein the solvent is selected from the group consisting of acetonitrile, isopropanol, and aqueous solutions of acetonitrile or isopropanol.
Clause 48. The method of any of clauses 40-47, further comprising the step of hydrating the extracted ophthalmic device with at least one aqueous solution.
Clause 49. The method of any of clauses 40-48, further comprising a step of sterilizing the ophthalmic device.
Clause 50. The method of clause 49, further comprising a step of irradiating the ophthalmic device using a femtosecond two photon laser either before or after sterilization.
Clause 51. The method of clause 50, wherein the irradiation step is performed on an implanted ophthalmic device.
Clause 52. The method of any of clauses 40-51, wherein the ophthalmic device is selected from the group consisting of an intraocular lens, phakic intraocular lens, contact lens, corneal inlay, corneal outlay, or corneal insert.
Clause 53. The method of clause 52, wherein the ophthalmic device is an intraocular lens.
Clause 54. A composition made by free radical polymerization of a reactive monomer mixture comprising: 2-((butylcarbamoyl)oxy)ethyl acrylate at 24-28 weight percent; 4-hydroxybutyl acrylate at 10 weight percent; tricyclo[5.2.1.0^{2,6}]decanedimethanol diacrylate at 1.5 weight percent; 3-((9-(dicyanomethylene)-9H-xanthen-3-yl)oxy)propyl methacrylate at 0.2 weight percent; bis(2,4,6-trimethylbenzoyl)-phenylphosphine oxide at 0.45 weight percent; ethylene glycol dicyclopentenyl ether acrylate at 55-59 weight percent; and n-hexyl acrylate at 4-6 weight percent; wherein the concentration of ethylene glycol dicyclopentenyl ether acrylate and n-hexyl acrylate vary, but the components of the reactive monomer mixture add up to 100 weight percent; wherein the composition exhibits a refractive index of at least 1.50 and an Abbe number of at least 50; and wherein the storage modulus is between 1 megapascal and 100 megapascals.
Clause 55. A composition made by free radical polymerization of a reactive monomer mixture comprising: 2-oxo-2-(decylamino)ethyl (meth)acrylate at 16-18 weight percent; 4-hydroxybutyl acrylate at 10 weight percent; tricyclo[5.2.1.0^{2,6}]decanedimethanol diacrylate at 1.5 weight percent; 3-((9-(dicyanomethylene)-9H-xanthen-3-yl)oxy)propyl methacrylate at 0.2 weight percent; bis(2,4,6-trimethylbenzoyl)-phenylphosphine oxide at 0.45 weight percent; ethylene glycol dicyclopentenyl ether acrylate at 65-67 weight percent; and n-hexyl acrylate at 5-7 weight percent; wherein the concentration of 2-oxo-2-(decylamino)ethyl (meth)acrylate, ethylene glycol dicyclopentenyl ether acrylate, and n-hexyl acrylate vary, but the components of the reactive monomer mixture add up to 100 weight percent; wherein the composition exhibits a refractive index of at least 1.50 and an Abbe number of at least 50; and wherein the storage modulus is between 1 megapascal and 100 megapascals.
Clause 56. An ophthalmic device made from the compositions of any of clauses 54-55.
Clause 57. The ophthalmic device of clause 56 wherein the ophthalmic device is an intraocular lens.
Clause 58. The ophthalmic device of clause 57 wherein the intraocular lens is coated.
Clause 59. A compound having the chemical structure depicted by Formula II, P_{g}-L-CONR¹R², wherein P_{g} is a polymerizable group, L is a linking group, and R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups.
Clause 60. The compound of clause 59, wherein the polymerizable group is a (meth)acrylate and the linking group is an unsubstituted alkylene group.
Clause 61. The compound of any of clauses 59-60, wherein the compound has the chemical structure depicted by Formula IV:

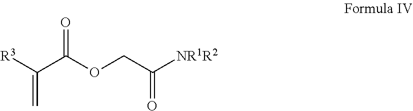

wherein R³ is H or methyl.
Clause 62. The compound of clause 61, wherein the compound is selected from the group consisting of 2-oxo-2-(methylamino)ethyl (meth)acrylate, 2-oxo-2-(ethylamino)ethyl (meth)acrylate, 2-oxo-2-(propylamino)ethyl (meth)acrylate, 2-oxo-2-(butylamino)ethyl (meth)acrylate, 2-oxo-2-(pentylamino)ethyl (meth)acrylate, 2-oxo-2-(hexylamino)ethyl (meth)acrylate, 2-oxo-2-(heptylamino)ethyl (meth)acrylate, 2-oxo-2-(octylamino)ethyl (meth)acrylate, 2-oxo-2-(nonylamino)ethyl (meth)acrylate, 2-oxo-2-(decylamino)ethyl (meth)acrylate, 2-oxo-2-(undecylamino)ethyl (meth)acrylate, 2-oxo-2-(dodecylamino)ethyl (meth)acrylate, 2-oxo-2-(tridecylamino)ethyl (meth)acrylate, 2-oxo-2-(tetradecylamino)ethyl (meth)acrylate, 2-oxo-2-((3-methoxypropyl)amino)ethyl (meth)acrylate, 2-oxo-2-((3-ethoxypropyl)amino)ethyl (meth)acrylate, 2-oxo-2-((cyclohexylmethyl)amino)ethyl (meth)acrylate, 2-oxo-2-(benzylamino)ethyl (meth)acrylate, 2-oxo-2-(phenethylamino)ethyl (meth)acrylate, 2-oxo-2-((thiophen-2-ylmethyl)amino)ethyl (meth)acrylate, 2-oxo-2-((2,3-dihydroxypropyl)amino)ethyl acrylate, and 2-(dimethylamino)-2-oxoethyl methacrylate.
Clause 63. A method of making any of the compounds of clauses 59-62, the method comprising: comprising (a) reacting a primary or secondary amine with methyl glycolate to form a N-alkyl-2-hydroxyacetamide or N-alkyl (R′)—N-alkyl (R″)-2-hydroxyacetamide and (b) reacting the N-alkyl-2-hydroxyacetamide or N-alkyl (R′)—N-alkyl (R″)-2-hydroxyacetamide with (meth)acryloyl chloride.
Clause 64. A composition made by free radical polymerization of a reactive monomer mixture comprising any of the compounds of clauses 59-62.
Clause 65. The composition of clause 64, wherein the reactive monomer mixture further comprises at least one of:
a) a cross-linking agent; and
b) an ethylene glycol dicyclopentenyl ether (meth)acrylate;
c) an aliphatic alkyl (meth)acrylate monomer;
d) a hydroxyalkyl (meth)acrylate monomer;
e) a free radical polymerization initiator;
f) at least one UV absorbing compound;
g) at least one UV/HEV absorbing compound;
h) a hydrophilic component selected from the group consisting of poly(ethylene glycol) (meth)acrylate, poly(ethylene glycol) methyl ether (meth)acrylate, poly(ethylene glycol) phenyl ether (meth)acrylate, and combinations thereof; and
i) at least one diluent.
Clause 66. An ophthalmic device comprising any of the compositions of clauses 64-65.
Clause 67. The ophthalmic device of clause 66, wherein the ophthalmic device is selected from the group consisting of an intraocular lens, phakic intraocular lens, contact lens, corneal inlay, corneal outlay, or corneal insert.
Clause 68. The ophthalmic device of clause 67, wherein the ophthalmic device is a contact lens.
Clause 69. A hydrogel composition made by free radical polymerization of a reactive monomer mixture comprising: a monomer having a chemical structure of Formula II, P_{g}-L-CONR¹R², wherein P_{g} is a polymerizable group, L is a linking group, and R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl and heteroarylalkyl groups; a cross-linking agent; and an initiator, wherein the hydrogel composition has a water contact between 10 weight percent and 90 weight percent, between 20 weight percent and 75 weight percent, or between 30 weight percent and 65 weight percent.
Clause 70. The hydrogel composition of clause 69, wherein the polymerizable group is a (meth)acrylate, the linking group is an alkylene group, and R¹ and R² are independently selected from H, alkyl, alkoxyalkyl, and hydroxyalkyl.
Clause 71. The hydrogel composition of clause 70, wherein the monomer has the chemical structure shown in Formula IV:

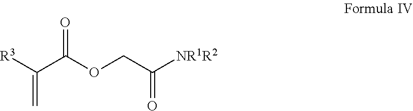

wherein R³ is H or methyl.
Clause 71. The hydrogel composition of any of clauses 69-71, further comprising a hydrophilic component.
Clause 72. The hydrogel composition of clause 71, wherein the hydrophilic component is selected from the group consisting of N, N-dimethyl acrylamide (DMA), N-vinylpyrrolidone (NVP), 2-hydroxyethyl methacrylate (HEMA), N-vinyl methacetamide (VMA), and N-vinyl N-methyl acetamide (NVA).
Clause 73. The hydrogel composition of any of clauses 69-72, further comprising a silicone-containing component, thereby forming a silicone hydrogel composition.
Clause 74. The hydrogel composition of clause 73, wherein the silicone-containing component has the chemical structure of Formula A:

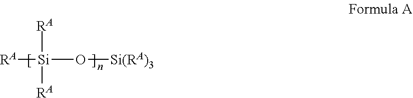

wherein at least one R^{A} is a group of Formula P_{g}-L-, wherein P_{g} is a polymerizable group and L is a linking group, and the remaining R^{A} are each independently:
j) P_{g}-L-;
k) C₁-C₁₆ alkyl optionally substituted with one or more hydroxy, amino, amido, oxa, carboxy, alkyl carboxy, carbonyl, alkoxy, amido, carbamate, carbonate, halo, phenyl, benzyl, or combinations thereof;
l) C₃-C₁₂ cycloalkyl optionally substituted with one or more alkyl, hydroxy, amino, amido, oxa, carbonyl, alkoxy, amido, carbamate, carbonate, halo, phenyl, benzyl, or combinations thereof;
m) a C₆-C₁₄ aryl optionally substituted with one or more alkyl, hydroxy, amino, amido, oxa, carboxy, alkyl carboxy, carbonyl, alkoxy, amido, carbamate, carbonate, halo, phenyl, benzyl, or combinations thereof;
n) halo;
o) alkoxy, cyclic alkoxy, or aryloxy;
p) siloxy;
q) alkyleneoxy-alkyl or alkoxy-alkyleneoxy-alkyl, such as polyethyleneoxyalkyl, polypropyleneoxyalkyl, or poly(ethyleneoxy-co-propyleneoxyalkyl); or
r) a monovalent siloxane chain comprising from 1 to 100 siloxane repeating units optionally substituted with alkyl, alkoxy, hydroxy, amino, oxa, carboxy, alkyl carboxy, alkoxy, amido, carbamate, halo or combinations thereof;
   wherein n is from 0 to 500 or from 0 to 200, or from 0 to 100, or from 0 to 20, where it is understood that when n is other than 0, n is a distribution having a mode equal to a stated value, and when n is 2 or more, the SiO units may carry the same or different RA substituents and if different RA substituents are present, the n groups may be in random or block configuration.
Clause 75. The hydrogel composition of clause 74, wherein the silicone-containing component is selected from the groups consisting of mono-n-butyl terminated monomethacryloxypropyl terminated polydimethylsiloxane (mPDMS), mono-n-butyl terminated mono-(2-hydroxy-3-methacryloxypropyloxy)-propyl terminated polydimethylsiloxane (OH-mPDMS), 3-(3-(1,1,1,3,5,5,5-heptamethyltrisiloxan-3-yl)propoxy)-2-hydroxypropyl methacrylate (SiMAA), and 3-(3-(1,5-di-tert-butyl-1,1,3,5,5-pentamethyltrisiloxan-3-yl)propoxy)-2-hydroxypropyl methacrylate (tBu-SiMAA).
Clause 76. The hydrogel composition of any of clauses 69-75, further comprising a polyamide.
Clause 77. The hydrogel composition of clause 76, wherein the polyamide is selected from the group consisting of polyvinylpyrrolidone (PVP), polyvinylmethyacetamide (PVMA), polydimethylacrylamide (PDMA), polyvinylacetamide (PNVA), and combinations thereof.
Clause 78. The hydrogel composition of any of clauses 69-77, further comprising an ultraviolet light absorbing compound.
Clause 79. The hydrogel composition of clause 78, wherein the ultraviolet light absorbing compound is 2-(2′-hydroxy-5-methacrylyloxyethylphenyl)-2H-benzotriazole (Norbloc).
Clause 80. The hydrogel composition of any of clauses 69-79, further comprising an UV/HEV absorbing compound.
Clause 81. The hydrogel composition of clause 80, wherein the UV-HEV absorbing compound has the chemical structures shown in Formula V and Formula VI:

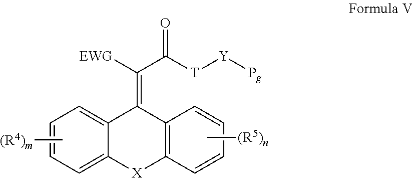

wherein:
m and n are independently 0, 1, 2, 3, or 4;
T is a bond, O, or NR′;
X is O, S, NR, SO, or SO₂;
Y is a linking group;
P_{g} is a polymerizable group;
R⁶ at each occurrence is independently H, C₁-C₆ alkyl, cycloalkyl, heterocycloalkyl, aryl, heteroaryl, or Y—P_{g};
R⁴ and R⁵, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR⁷R⁸, or benzyl, wherein R⁷ and R⁸ are independently H or C₁-C₆ alkyl, or two adjacent R⁴ or R⁵ groups, together with the carbon atoms to which they are attached, combine to form a cycloalkyl or aryl ring; and EWG is an electron withdrawing group, preferably cyano;

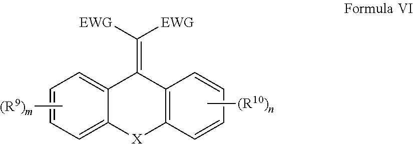

wherein:
m and n are independently 0, 1, 2, 3, or 4;
X is O, S, NR¹¹, SO, or SO₂;
R¹¹ at each occurrence is independently H, C₁-C₆ alkyl, cycloalkyl, heterocycloalkyl, aryl, heteroaryl, or Y—P_{g}, wherein Y is a linking group and P_{g} is a polymerizable group;
R⁹ and R¹⁰, when present, are independently at each occurrence C₁-C₆ alkyl, C₁-C₆ alkoxy, C₁-C₆ thioalkyl, C₃-C₇ cycloalkyl, aryl (preferably unsubstituted phenyl or phenyl substituted with alkyl or halo), halo, hydroxy, amino, NR¹²R¹³, or benzyl, wherein R¹² and R¹³ are independently H or C₁-C₆ alkyl, two adjacent R⁹ or R¹⁰ groups, together with the carbon atoms to which they are attached, combine to form a cycloalkyl or aryl ring, Y—P_{g}, or T-Y—P_{g}, wherein T is a bond, O, or NR¹¹; and EWG is an electron withdrawing group, preferably cyano.
Clause 80. The hydrogel composition of clause 79, wherein the UV/HEV absorbing compound is selected from the group consisting of 2-(2-cyano-2-(9H-thioxanthen-9-ylidene)acetamido)ethyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 2-(2-cyano-2-(2-methoxy-10-propylacridin-9(10H)-ylidene)acetamido)ethyl methacrylate, 2-(2-cyano-2-(2-methoxy-10-butylacridin-9(10H)-ylidene)acetamido)ethyl methacrylate, 3-((9-(dicyanomethylene)-9H-xanthen-3-yl)oxy)propyl methacrylate, or any combination thereof.
Clause 81. The hydrogel composition of any of clauses 69-80, wherein the cross-linking agent is selected from the group consisting of ethylene glycol dimethacrylate, tetraethylene glycol dimethacrylate, trimethylolpropane trimethacrylate, methylene bisacrylamide, triallyl cyanurate, and combinations thereof.
Clause 82. The hydrogel composition of any of clauses 69-81, wherein the initiator is selected from the group consisting of thermal initiators, photo-initiators, and combinations thereof.
Clause 83. The hydrogel composition of clause 82, wherein the photo-initiator is selected from the group consisting of aromatic alpha-hydroxy ketones, alkoxyoxybenzoins, acetophenones, monoacylphosphine oxides, bisacylphosphine oxides, and combinations thereof.
Clause 84. An ophthalmic device made from any of the hydrogel compositions of clauses 69-83.
Clause 85. The ophthalmic device of clause 84 wherein the ophthalmic device is a contact lens.

Some embodiments of the disclosure will now be described in detail in the following Examples.

### EXAMPLES

Some embodiments of the disclosure will now be described in detail in the following Examples.

### IOL Test Methods

Unless otherwise noted, IOL test samples for refractive index, Abbe number, water content and glass transition temperature were polymer buttons that had been extracted and dried.

Refractive Index Test Method: Refractive index (RI) was measured using an Anton Paar Abbemat WR-wavelength refractometer. The instrument was equilibrated at either 25° C. or 35° C. for a minimum of 1 hour prior to use. The measurement wavelength was set at 589.3 nanometers. Using a pair of tweezers, the sample was placed on the quartz plate. The instrument lid was closed, and a custom-made metal tube weighing 1400 grams was placed on the lid to maintain constant pressure. The refractive index was recorded after 60 seconds of dwell time. Measurements were performed on three polymer buttons, and the average was reported. In some examples, where it is noted, measurements were performed on both sides of the three polymer buttons, and the average of the six measurements was reported.

Abbe Number Test Method: Following the steps for measuring the refractive index at 589.3 nm, the refractive index at 486.1 nanometers and 656.3 nanometers were determined. Measurements were performed on three polymer buttons, and for each polymer button, the refractive index measurements at all three wavelengths were completed before measuring the next replicate. The Abbe number was calculated as follows: Abbe number V=(n_{D}−1)/(n_{F}−n_{C}), wherein n_{D}, n_{F} and n_{C} are the refractive indices of the material at the wavelengths of the Fraunhofer D, F and C spectral lines (589.3 nanometers, 486.1 nanometers and 656.3 nanometers, respectively). The average of the three measurements was reported. In some examples, where it is noted, measurements were performed on both sides of the three polymer buttons, and the average of the six measurements was reported.

Water Content Test Method: The water content (WC) was determined gravimetrically. In this method, three dry polymer disks were individually weighed and transferred to individual glass scintillation vials using sharp-tipped metal tweezers. About 10 mL of HPLC grade water was transferred into each vial, and the samples were incubated at 37° C. for 14 days. After incubation, the polymer disks were removed from the vials using a sharp-tipped metal tweezers and briefly blotted on all sides (flat surfaces and edge) using lint-free blotting paper to remove surface/excess water. Using a dry tweezers, each polymer disk was placed in a tared weighing pan and weighed individually. The water content of the polymer disk was calculated as follows: (% WC)=(wet weight−dry weight)/wet weight×100. The average and standard deviation of the water content were calculated, and the average value reported as the percent water content of the disk.

Ultraviolet-Visible Spectroscopy Test Methods: Ultraviolet-visible spectra of compounds in solution were measured on a Perkin Elmer Lambda 45 or an Agilent Cary 6000i UV/VIS scanning spectrometer. The instrument was thermally equilibrated for at least thirty minutes prior to use. For the Perkin Elmer instrument, the scan range was 200-800 nanometers; the scan speed was 960 nm per minute; the slit width was 4 nm; the mode was set on transmission or absorbance; and baseline correction was selected. For the Cary instrument, the scan range was 200-800 nm; the scan speed was 600 nm/min; the slit width was 2 nm; the mode was transmission or absorbance; and baseline correction was selected It is important to ensure that the outside surfaces of the cuvette are completely clean and dry and that no air bubbles are present in the cuvette. Repeatability of the measurement is improved when the reference cuvette and its lens holder remain constant and when all samples use the same sample cuvette and its lens holder, making sure that both cuvettes are properly inserted into the instrument.

Ultraviolet-visible spectra of disks formed from the claimed compositions were measured on a Perkin Elmer Lambda 45 UV/VIS or an Agilent Cary 6000i UV/VIS scanning spectrometer as described above using a custom-made, adjustable holder to position the disk in the beam. The custom-made, adjustable holders were V shaped and allowed the disks to slide into place. Baseline correction was performed using empty custom-made, adjustable holders. For obtaining UV-VIS spectra on wet disks, another custom-made, adjustable holder was used to hold cuvettes engineered to hold the disk in the quartz cuvette in the location through which the incident light beam traverses. Baseline correction was performed using custom-made, adjustable holders and empty cuvettes (solvent, no disks). To ensure that the thickness of the samples is constant, all lenses were made using identical molds. Absorbance or transmission spectra are obtained by averaging three individual disk data.

Glass Transition Temperature Test Method: Because of the thickness and/or brittleness of the polymer disks, test samples were cut from the center of the polymer disk using a razor blade. The samples could not be punched out as with a thin film. Test samples were analyzed (in duplicate) on a DSC Q2000 TA instrument at heating rates of 10° C./minute and cooling rates of 5° C./minute under a nitrogen gas atmosphere. The glass transition temperatures were determined from the second heating scans unless noted otherwise.

Dynamic Mechanical Analysis (DMA): Dynamic mechanical analysis was performed using a solids analyzer model RSA G2 from TA Instruments in tension mode. Rectangular specimens were cut from the polymer disks having a width of about 3 millimeters, a length of about 5 millimeters, and a thickness of about 0.75 millimeters. The storage modulus (E′) was determined at 22° C. in the elastic regime, straining at one Hertz. Tan delta (E″/E′) was also determined by a temperature sweep analysis from 10° C. to 40° C. with a temperature sweep rate of 2° C./minute and a strain frequency of one Hertz, wherein E″ is the loss modulus. The units of E′ and E″ are megapascals (MPa). Tan delta is reported as the temperature (° C.) of maximum damping (tan δmax) and is used to estimate the glass transition temperature of the material.

### Contact Lens Test Methods

Unless otherwise noted, the test methods used to characterize contact lens test samples are described below. Standard deviations are shown in parentheses or as ± in the tables.

Water content (WC) was measured gravimetrically. Lenses were equilibrated in packing solution for 24 hours. Each of three test lenses was removed from packing solution using a sponge tipped swab and placed on blotting wipes which have been dampened with packing solution. Both sides of the lens were contacted with the wipe. Using tweezers, the test lenses were placed in a tared weighing pan and weighed. The two more sets of samples were prepared and weighed. All weight measurements were done in triplicate, and the average of those values was used in the calculations. The wet weight was defined as the combined weight of the pan and wet lenses minus the weight of the weighing pan alone.

The dry weight was measured by placing the sample pans in a vacuum oven which has been preheated to 60° C. for 30 minutes. Vacuum was applied until the pressure reached at least 1 inch of Hg; lower pressures are allowed. The vacuum valve and pump were turned off, and the lenses were dried for at least 12 hours, typically overnight. The purge valve was opened allowing dry air or dry nitrogen gas to enter. The oven was allowed reach atmospheric pressure. The pans were removed and weighed. The dry weight was defined as the combined weight of the pan and dry lenses minus the weight of the weighing pan alone. The water content of the test lens was calculated as follows: % water content=(wet weight−dry weight)/wet weight×100. The average and standard deviation of the water content were calculated, and the average value reported as the percent water content of the test lens.

The refractive index (RI) of a contact lens was measured by a Leica ARIAS 500 Abbe refractometer in manual mode or by a Reichert ARIAS 500 Abbe refractometer in automatic mode with a prism gap distance of 100 microns. The instrument was calibrated using deionized water at 20° C. (±0.2° C.). The prism assembly was opened, and the test lens was placed on the lower prism between the magnetic dots closest to the light source. If the prism was dry, a few drops of saline were applied to the bottom prism. The front curve of the lens was against the bottom prism. The prism assembly was then closed. After adjusting the controls so that the shadow line appeared in the reticle field, the refractive index was measured. The RI measurement was made on five test lenses. The average RI calculated from the five measurements was recorded as the refractive index as well as its standard deviation.

Haze was measured by placing a hydrated test lens in borate buffered saline in a clear glass cell at ambient temperature above a flat black background, illuminating from below with a fiber optic lamp (Dolan-Jenner PL-900 fiber optic light with 0.5″ diameter light guide) at an angle 660 normal to the lens cell, and capturing an image of the lens from above, normal to the lens cell with a video camera (DVC 1300C:19130 RGB camera or equivalent equipped with a suitable zoom camera lens) placed 14 mm above the lens holder. The background scatter was subtracted from the scatter of the test lens by subtracting an image of a blank cell with borate buffered saline (baseline) using EPIX XCAP V 3.8 software. The value for high end scatter (frosted glass) was obtained by adjusting the light intensity to be between 900 to 910 mean grayscale. The value of the background scatter (BS) was measured using a saline filled glass cell. The subtracted scattered light image was quantitatively analyzed, by integrating over the central 10 mm of the lens, and then comparing to a frosted glass standard. The light intensity/power setting was adjusted to achieve a mean grayscale value in the range of 900-910 for the frosted glass standard; at this setting, the baseline mean grayscale value was in the range of 50-70. The mean grayscale values of the baseline and frosted glass standard were recorded and used to create a scale from zero to 100, respectively. In the grayscale analysis, the mean and standard deviations of the baseline, frosted glass, and every test lens were recorded. For each lens, a scaled value was calculated according to the equation: scaled value equals the mean grayscale value (lens minus baseline) divided by the mean grayscale value (frosted glass minus baseline) times by 100%. Three to five test lenses were analyzed, and the results were averaged and reported as % Haze.

Oxygen permeability (Dₖ) was determined by the polarographic method generally described in ISO 9913-1:1996 and ISO 18369-4:2006, but with the following modifications. The measurement was conducted at an environment containing 2.1% oxygen created by equipping the test chamber with nitrogen and air inputs set at the appropriate ratio, for example, 1800 mL/min of nitrogen and 200 mL/min of air. The t/Dk was calculated using the adjusted oxygen concentration. Borate buffered saline was used. The dark current was measured by using a pure humidified nitrogen environment instead of applying MMA lenses. The lenses were not blotted before measuring. Four lenses were stacked instead of using lenses of various thickness (t) measured in centimeters. A curved sensor was used in place of a flat sensor; radius was 7.8 mm. The calculations for a 7.8 mm radius sensor and 10% (v/v) air flow were as follows:

Dₖ/t=(measured current−dark current)×(2.97×10−8 mL O₂/(μA-sec-cm²-mm Hg)

The edge correction was related to the Dₖ of the material.

For all Dₖ values less than 90 barrers:

t/Dₖ (edge corrected)=(1+(5.88×t))×(t/Dₖ)

For Dₖ values between 90 and 300 barrers:

t/Dₖ (edge corrected)=(1+(3.56×t))×(t/Dₖ)

For Dₖ values greater than 300 barrers:

t/Dₖ (edge corrected)=(1+(3.16×t))×(t/Dₖ)

Non-edge corrected Dₖ was calculated from the reciprocal of the slope obtained from the linear regression analysis of the data wherein the x variable is the center thickness in centimeters and the y variable is the t/Dₖ value. On the other hand, edge corrected Dₖ (EC Dₖ) was calculated from the reciprocal of the slope obtained from the linear regression analysis of the data wherein the x variable is the center thickness in centimeters and the y variable is the edge corrected t/Dₖ value. The resulting Dₖ value was reported in barrers.

Wettability of lenses was determined using a sessile drop technique using KRUSS DSA-100 ™ instrument at room temperature and using deionized water as probe solution (Sessile Drop). The lenses to be tested were rinsed in deionized water to remove carry over from packing solution. Each test lens was placed on blotting lint free wipes which are dampened with packing solution. Both sides of the lens were contacted with the wipe to remove surface water without drying the lens. To ensure proper flattening, lenses were placed “bowl side down” on the convex surface of contact lens plastic molds. The plastic mold and the lens were placed in the sessile drop instrument holder, ensuring proper central syringe alignment. A 3 to 4 microliter drop of deionized water was formed on the syringe tip using DSA 100-Drop Shape Analysis software ensuring the liquid drop was hanging away from the lens. The drop was released smoothly on the lens surface by moving the needle down. The needle was withdrawn away immediately after dispensing the drop. The liquid drop was allowed to equilibrate on the lens for 5 to 10 seconds, and the contact angle was measured between the drop image and the lens surface. Typically, three to five lenses were evaluated, and the average contact angle was reported.

Lens wettability was also assessed by measuring dynamic contact angles. The dynamic contact angle was determined by a Wilhelmy plate method using a Cahn DCA-315 instrument at room temperature and using deionized water as the probe solution (Cahn DCA). The experiment was performed by dipping the lens specimen of known parameter into the packing solution of known surface tension while measuring the force exerted on the sample due to wetting by a sensitive balance. The advancing contact angle of the packing solution on the lens is determined from the force data collected during sample dipping. The receding contact angle is likewise determined from force data while withdrawing the sample from the liquid. The Wilhelmy plate method is based on the following formula: Fg=γρ cos θ−B, wherein F=the wetting force between the liquid and the lens (mg), g=gravitational acceleration (980.665 cm/sec²), γ=surface tension of probe liquid (dyne/cm), ρ=the perimeter of the contact lens at the liquid/lens meniscus (cm), θ=the dynamic contact angle (degree), and B=buoyancy (mg). B is zero at the zero depth of immersion. Four test strips were cut from the central area of the contact lens. Each strip was approximately 5 mm in width and equilibrated in packing solution. Then, each sample was cycled four times, and the results were averaged to obtain the advancing and receding contact angles of the lens. Advancing and receding dynamic contact angles are listed in the tables in that order. However, in the claims, only the dynamic advancing contact angles are used.

The mechanical properties of the contact lenses were measured by using a tensile testing machine such as an Instron model 1122 or 5542 equipped with a load cell and pneumatic grip controls. Minus one diopter lens was the preferred lens geometry because of its central uniform thickness profile. A dog-bone shaped sample cut from a −1.00 diopter power lens having a 0.522 inch length, 0.276 inch “ear” width and 0.213 inch “neck” width was loaded into the grips and elongated at a constant rate of strain of 2 inches per minute until it breaks. The center thickness of the dog-bone sample was measured using an electronic thickness gauge prior to testing. The initial gauge length of the sample (Lₒ) and sample length at break (L_{f}) were measured. At least five specimens of each composition were measured, and the average values were used to calculate the percent elongation to break: percent elongation=((L_{f}−Lₒ)/Lₒ)×100. The tensile modulus (M) was calculated as the slope of the initial linear portion of the stress-strain curve; the units of modulus are pounds per square inch or psi. The tensile strength (TS) was calculated from the peak load and the original cross-sectional area: tensile strength=peak load divided by the original cross-sectional area; the units of tensile strength are psi. Toughness was calculated from the energy to break and the original volume of the sample: toughness=energy to break divided by the original sample volume; the units of toughness are in-lbs/in³. The elongation to break (ETB) was also recorded as the percent strain at break.

Polymer molecular weights were determined by Size Exclusion Chromatography with Multi-Angle Light Scattering (SEC-MALS). A typical SEC-MALS setup employed a suitable solvent such as 1-propanol (or THF) with (or without) 10 mM LiBr (or another commonly used salt) as the mobile phase at a flow rate of 0.6 mL/min at 65° C. Three Tosoh Biosciences TSK-gel columns in series were used [SuperAW3000 4 um, 6.0 mm ID×15 cm (PEO/DMF Exclusion Limit=60,000 g/mole), SuperAW4000 6 um, 6.0 mm ID×15 cm (PEO/DMF Exclusion Limit=400,000 g/mole) and a SuperAW5000 7 um, 6.0 mm ID×15 cm (PEO/DMF Exclusion Limit=4,000,000 g/mole)] with an online Agilent 1200 UV/VIS diode array detector, a Wyatt Optilab rEX interferometric refractometer, and a Wyatt mini-DAWN Treos multiangle laser scattering (MALS) detector (λ=658 nm). A dη/dc value of 0.0.074 mL/g at 30° C. (λ=658 nm) was used for absolute molecular weight determination. Absolute molecular weights and polydispersity data were calculated using the Wyatt ASTRA 6.1.1.17 SEC/LS software package.

### General Abbreviations

The following abbreviations will be used throughout the Examples and have the following meanings:
IOL: intraocular lens(es)
CL: contact lens(es)
TL03 lights: Phillips TLK 40W/03 bulbs
LED: light emitting diode
RMM: reactive monomer mixture(s)
RI (25): refractive index measured at 25° C.
RI (35): refractive index measured at 35° C.
Abbe #(25): Abbe number measured at 25° C.
Abbe #(35): Abbe number measured at 35° C.
T_{g}: glass transition temperature (° C.) as determined by differential scanning calorimetry (DSC)
WC: water content
Wt. %: weight percent
UV-VIS: ultraviolet-visible (spectroscopy)
UV-HEV or UV/HEV: ultraviolet and high energy visible (light)
NMR: nuclear magnetic resonance (spectroscopy)
TLC: thin layer chromatography
h: hour(s)
RT: room temperature
mm: millimeter(s)
cm: centimeter(s)
μm: micrometer(s)
nm: nanometer(s)
mL: milliliter(s)
N: normal (equivalents/liter)
M: molar (moles/liter)
mM: millimolar (millimoles/liter)
μL: microliter(s)
mW: milliwatt(s)
g/mol: grams/mole
Mₙ: number average molecular weight
Da or Dalton(s): gram(s)/mole
kDa: kilodalton(s)
λ: wavelength
rpm: revolutions per minute
psi: pounds per square inch
BC: base curve plastic mold made of PP, TT, Z, or blends thereof
FC: front curve plastic mold made of PP, TT, Z, or blends thereof
PP: polypropylene which is the homopolymer of propylene
TT: Tuftec which is a hydrogenated styrene butadiene block copolymer (Asahi Kasei Chemicals)
Z: Zeonor which is a polycycloolefin thermoplastic polymer (Nippon Zeon Co Ltd)

### Solvent Abbreviations

ACN: acetonitrile
EtOAc: ethyl acetate
MeOH: methanol
DCM: dichloromethane or methylene chloride
Et₃N: triethylamine
3E3P: 3-ethyl 3-pentanol
D3O: 3,7-dimethyl-3-octanol (Vigon)
DIW: deionized water
IPA: isopropyl alcohol
PG: 1,2-propylene glycol
PBS: phosphate buffered saline
PS: Borate Buffered Packing Solution: 18.52 grams (300 mmol) of boric acid, 3.7 grams (9.7 mmol) of sodium borate decahydrate, and 28 grams (197 mmol) of sodium sulfate were dissolved in enough deionized water to fill a 2-liter volumetric flask.
BAGE: Boric Acid Glycerol Ester (molar ratio of boric acid to glycerol was 1:2) 299.3 grams (mol) of glycerol and 99.8 grams (mol) of boric acid were dissolved in 1247.4 grams of a 5% (w/w) aqueous ethylenediaminetetraacetic acid solution in a suitable reactor and then heated with stirring to 90-94° C. under mild vacuum (2-6 torr) for 4-5 hours and allowed to cool down to room temperature.

### RMM Component Abbreviations

HEMA: 2-hydroxyethyl methacrylate (Bimax)
GMMA: 2,3-dihydroxypropyl methacrylate (Polysciences)
DMA: N, N-dimethylacrylamide (Jarchem)
MMA: methacrylic acid (Acros)
PVP K90: poly(N-vinylpyrrolidone) (ISP Ashland)
SiMAA: 2-propenoic acid, 2-methyl-2-hydroxy-3-[3-[1,3,3,3-tetramethyl-1-[(trimethylsilyl)oxy]disiloxanyl]propoxy]propyl ester (Toray) or 3-(3-(1,1,1,3,5,5,5-heptamethyltrisiloxan-3-yl)propoxy)-2-hydroxypropyl methacrylate

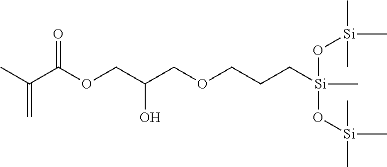 [SMILES: C=C(C)C(=O)OCC(O)COCCC[Si](C)(O[Si](C)(C)C)O[Si](C)(C)C]

tBu-SiMAA: 3-(3-(1,5-di-tert-butyl-1,1,3,5,5-pentamethyltrisiloxan-3-yl)propoxy)-2-hydroxypropyl methacrylate (Shin Etsu)

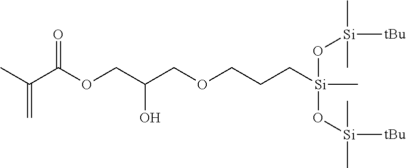 [SMILES: C=C(C)C(=O)OCC(O)COCCC[Si](C)(O[Si](C)(C)C(C)(C)C)O[Si](C)(C)C(C)(C)C]

mPDMS: mono-n-butyl terminated monomethacryloxypropyl terminated polydimethylsiloxane (Mₙ=800-1500 grams/mole) (Gelest)
HO-mPDMS: mono-n-butyl terminated mono-(2-hydroxy-3-methacryloxypropyloxy)-propyl terminated polydimethylsiloxane (Mₙ=400 to 1400 grams/mole) (Ortec or DSM-Polymer Technology Group)
OH-mPDMS (n=4):

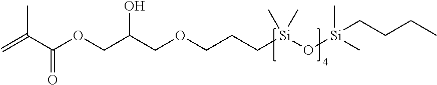

OH-mPDMS (n=14) which is an oligomeric macromer having a number average degree of polymerization DP=14 (Ortec):

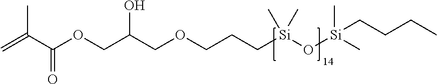

NHA: n-hexyl acrylate [CAS 2499-95-8] (Sigma-Aldrich)
NBA: n-butyl acrylate
HBA: 4-hydroxybutyl acrylate [CAS 2478-10-6] (TCI or BASF)
E2EA: 2-(2-ethoxyethoxy)ethyl acrylate
PEA: 2-phenylethyl acrylate [CAS 3530-36-7] (MPD)
PEMA: 2-phenylethyl methacrylate [CAS 3683-12-3]
PPA: 3-phenylpropyl acrylate [CAS 85909-41-7]
CHA: cyclohexyl acrylate [CAS 3066-71-5] (TCI or Alfa Aesar)
CHMA: cyclohexylmethyl acrylate

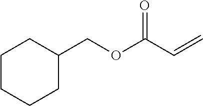

CHEA: 2-cyclohexylethyl acrylate

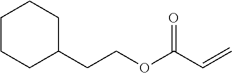

CHPA: 3-cyclohexylpropyl acrylate

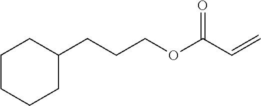

EGDCA: Ethylene glycol dicyclopentenyl ether acrylate [CAS 65983-31-5] (Sigma-Aldrich)

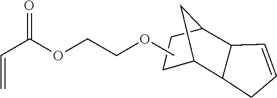

BCHA: ((1R,2S,4R)-bicyclo[2.2.1]hept-5-en-2-yl)methyl acrylate or cyclol acrylate or [(1S,4S)-2-bicyclo[2.2.1]hept-5-enyl]methyl prop-2-enoate [CAS 95-39-6] (Monomer-Polymer and DAJAC Labs Inc.)

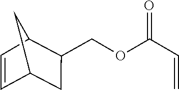

CAA: cinnamyl acrylate

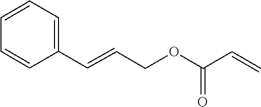

TCDA: Tricyclo[5.2.1.02,6]decanedimethanol diacrylate or dimethylol tricyclo decane diacrylate [CAS 42594-17-2] (Sigma-Aldrich or Kyoeisha Chemical Co.)

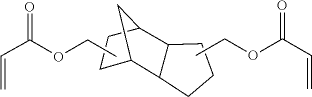

mPEG 300: poly(ethylene glycol) methyl ether methacrylate (Mn=300 grams/mole) (Sigma-Aldrich)

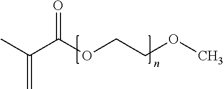

PEG-OH 200: poly(ethylene glycol) methacrylate (Polysciences; molecular weight of the PEG block is 200 grams/mole)
PEG-OH 360: poly(ethylene glycol) methacrylate (Mn=360 grams/mole) (Sigma-Aldrich)

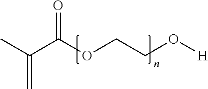

PEG-OH—N2:

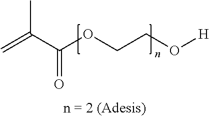

PEG-OH—N3:

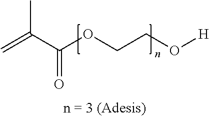

PEG-OH—N4:

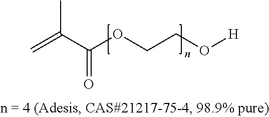

PEG-OH—N5:

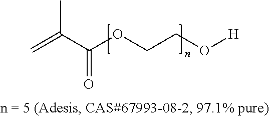

PEG-OH—N6:

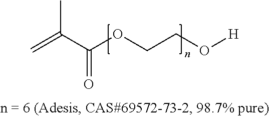

PEG-OH—N7:

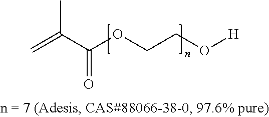

PEG-OH—N8:

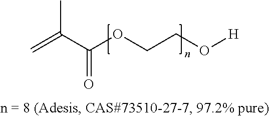

PEG-OH-MIX1:

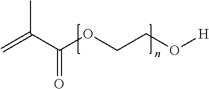

Mixture of oligomers PEG-OH—N4, PEG-OH—N5, PEG-OH—N6, PEG-OH—N7, and PEG-OH—N8 formulated to exhibit a Poissen's molecular weight distribution centered around PEG-OH—N6.
PEG-OH-MIX2:

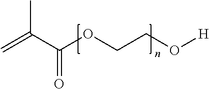

Mixture of oligomers PEG-OH—N2, PEG-OH—N3, PEG-OH—N4, PEG-OH—N5, PEG-OH—N6, PEG-OH—N7, and PEG-OH—N8 formulated to exhibit a Poissen's molecular weight distribution centered around PEG-OH—N5.
PEPEA: poly(ethylene glycol) phenyl ether acrylate [CAS #56641-05-5, Mₙ=324 grams/mole] (Sigma-Aldrich)

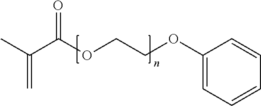

BCEA: 2-((butylcarbamoyl)oxy)ethyl acrylate (Sigma-Aldrich)

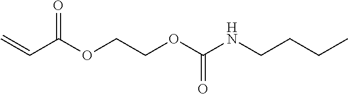

OPEA: 2-oxo-2-(pentylamino)ethyl acrylate

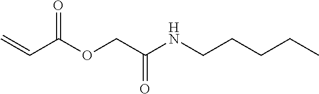

MOPEA: 2-oxo-2-((3-methoxypropyl)amino)ethyl acrylate

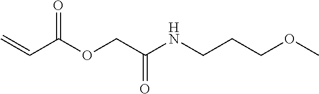

OAOEA: 2-oxo-2-(octylamino)ethyl acrylate

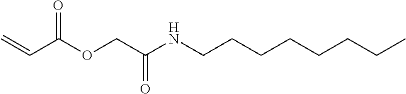 [SMILES: C=CC(=O)OCC(=O)NCCCCCCCC]

DAOEA: 2-oxo-2-(decylamino)ethyl acrylate

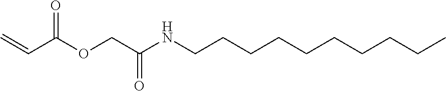 [SMILES: C=CC(=O)OCC(=O)NCCCCCCCCCC]

CHOPE: 2-oxo-2-((cyclohexylmethyl)amino)ethyl acrylate

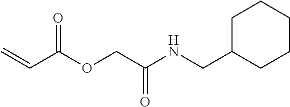 [SMILES: C=CC(=O)OCC(=O)NCC1CCCCC1]

BZOEA: 2-oxo-2-(benzylamino)ethyl acrylate

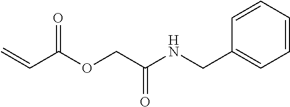 [SMILES: C=CC(=O)OCC(=O)NCc1ccccc1]

OPEEA: 2-oxo-2-(phenethylamino)ethyl acrylate

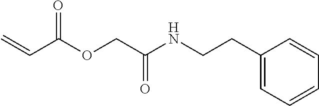 [SMILES: C=CC(=O)OCC(=O)NCCc1ccccc1]

OTPEA: 2-oxo-2-((thiophen-2-ylmethyl)amino)ethyl acrylate

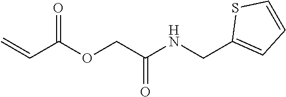

OPAEA: 2-oxo-2-((2-pentanamidoethyl)amino)ethyl acrylate

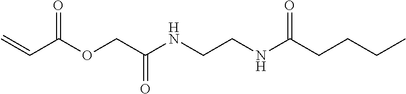 [SMILES: C=CC(=O)OCC(=O)NCCNC(=O)CCCC]

DHOEMA: 2-oxo-2-((2,3-dihydroxypropyl)amino)ethyl methacrylate

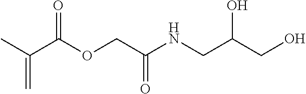

DMAOEMA: 2-(dimethylamino)-2-oxoethyl methacrylate

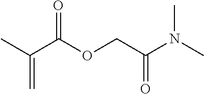

EGDMA: ethylene glycol dimethacrylate (Esstech)
TMPTMA: trimethylolpropane trimethacrylate (Esstech)
TRGDMA: triethylene glycol dimethacrylate (Sigma Aldrich)
TEGDMA: tetraethylene glycol dimethacrylate (Esstech)
Tegomer MA: bis-3-methacryloxy-2-hydroxypropyloxypropyl polydimethylsiloxane (Mₙ=2000 grams/mole, n=20) (Shin Etsu)

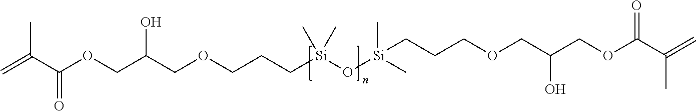

BHPMA-PDMS: bis-3-methacryloxy-2-hydroxypropyloxypropyl polydimethylsiloxane (Mₙ=2100 grams/mole, n=21) (Shin Etsu)

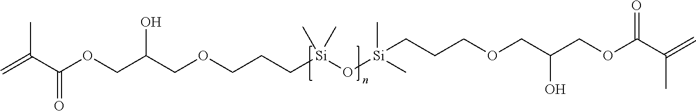

TCDA: Tricyclo[5.2.1.02,6]decanedimethanol diacrylate or dimethylol tricyclo decane diacrylate [CAS 42594-17-2] (Sigma-Aldrich or Kyoeisha Chemical Co.)

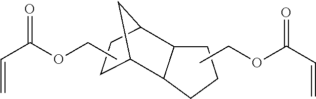

Norbloc: 2-(2′-hydroxy-5-methacrylyloxyethylphenyl)-2H-benzotriazole (Janssen)
Blue HEMA: 1-amino-4-[3-(4-(2-methacryloyloxy-ethoxy)-6-chlorotriazin-2-ylamino)-4-sulfophenylamino]anthraquinone-2-sulfonic acid, as described in U.S. Pat. No. 5,944,853
RB247: 1,4-Bis[2-methacryloxyethylamino]-9,10-anthraquinone (CAS #109561-07-1)
UVB: 3-(3-(tert-butyl)-5-(5-chloro-2H-benzo[d][1,2,3]triazol-2-yl)-4-hydroxyphenyl)-propyl methacrylate or 2-Methylacrylic acid, 3-[3-tert-butyl-5-(5-chlorobenzotriazol-2-yl)-4-hydroxyphenyl]-propyl ester (Adesis)

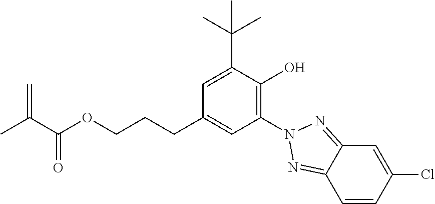 [SMILES: C=C(C)C(=O)OCCCc1cc(-n2nc3ccc(Cl)cc3n2)c(O)c(C(C)(C)C)c1]

HEVB: 2-(2-cyano-2-(9H-thioxanthen-9-ylidene)acetamido)ethyl methacrylate

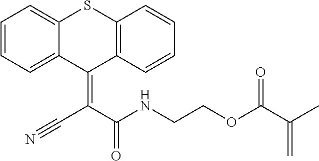 [SMILES: C=C(C)C(=O)OCCNC(=O)C(C#N)=C1c2ccccc2Sc2ccccc21]

HEVC: 3-((9-(dicyanomethylene)-9H-xanthen-3-yl)oxy)propyl methacrylate

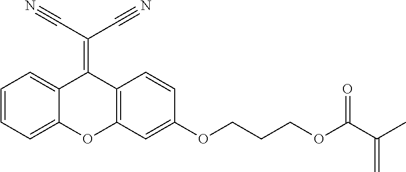 [SMILES: C=C(C)C(=O)OCCCOc1ccc2c(c1)Oc1ccccc1C2=C(C#N)C#N]

Omnirad 403: bis(2,6-dimethoxybenzoyl)-2,4,4-trimethylpentylphosphine oxide (IGM Resins)
Omnirad 1173: 2-hydroxy-2-methyl-1-phenylpropanone (IGM Resins)
Omnirad 1700: mixture of 25 weight % Omnirad 403 and 75 weight % Omnirad 1173 (IGM Resins)
Omnirad 1870: mixture of 70 weight % Omnirad 403 and 30 weight % Omnirad 1173 (IGM Resins)
Omnirad 819: bis(2,4,6-trimethylbenzoyl)-phenylphosphine oxide [CAS 162881-26-7](IGM Resins)
AIBN: azobisisobutyronitrile [CAS 78-67-1]
BHT: butylated hydroxytoluene, also known as dibutylhydroxytoluene

### Preparations

### Preparation 1: Synthesis of 2-(2-cyano-2-(9H-thioxanthen-9-ylidene)acetamido)-ethyl methacrylate (HEVB) is shown in Scheme 1

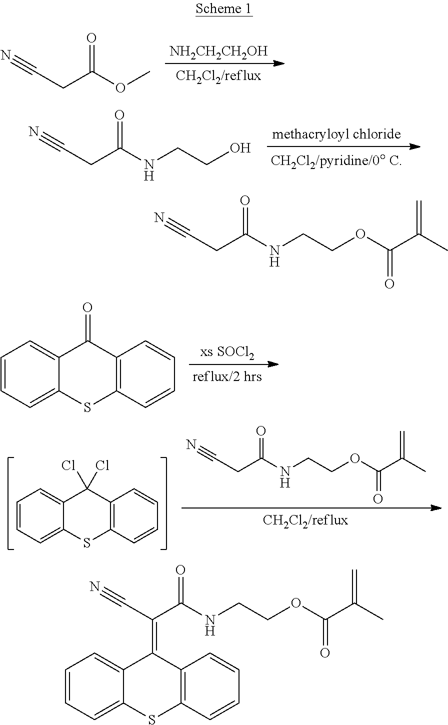

Methyl cyanoacetate (40 grams, 0.4037 mole) and 25 mL of dichloromethane were stirred in a 3 neck, 500 mL round bottom flask under equipped with a reflux condenser under a nitrogen environment. 2-aminoethanol (23.8 grams, 0.3897 mole, ˜0.97 eq.) was added to the solution via an addition funnel, after which the temperature rose, and the methylene chloride began to reflux. After the exotherm ceased, external heat was applied to continue a gentle reflux for a total of two hours, after which no ethanolamine was observed by thin layer chromatography.

The reaction may also be conducted at room temperature and is complete within a few hours. The mixture was cooled to room temperature and all the methylene chloride was evaporated at reduced pressure. The residual oil was washed three times with 50 mL of ethyl acetate to remove unreacted starting material and non-polar impurities. The residual ethyl acetate was then removed under reduced pressure, and the resulting oil was used for acylation without any further purification.

The crude N-2-hydroxyethylacetamide derivative was dissolved in 150 mL of dichloromethane containing 40 grams of pyridine (˜0.5 mole) in a three-neck round bottom flask equipped with a reflux condenser, an addition funnel, and a magnetic stirring bar. The flask was immersed in an ice bath and allowed to cool down to around 0° C. Methacryloyl chloride (45.76 grams, ˜0.44 mole) was added dropwise from the addition funnel, and the resulting reaction mixture was allowed to warm up to room temperature while constantly stirring the system. Methanol (20 mL) was the added to the flask to quench any unreacted methacryloyl chloride. The volatile components were removed by rotary evaporation under reduced pressure, and the crude product dissolved in 800 mL of dilute aqueous HCl. The resulting aqueous solution was extracted three times with 100 mL of hexanes in a separatory funnel to remove any non-polar impurities. The organic layers were discarded. Sodium chloride was added to the aqueous layer which was then extracted three times with 300 mL of ethyl acetate. About 50 milligrams of BHT were added to the combined organic fractions as an inhibitor, and the ethyl acetate removed by rotary evaporation under reduced pressure. The crude product crystalized out of solution during solvent removal. When about 100 mL of ethyl acetate was left in the flask, 250 mL of hexanes was added, and the crude product was isolated by vacuum filtration using a fritted glass funnel. Thin layer chromatography indicated the presence of a single compound. The filter cake was washed two times with 150 mL of hexanes and then vacuum dried at 40° C., yielding 53 grams (about 70% yield) of 2-(2-cyanoacetamido)ethyl methacrylate. ¹H NMR (500 MHz, CDCl₃) δ 1.93 (3H, s, CH₃), 3.36 (2H, s, CNCH₂), 3.60 (2H, dd, CH₂NH), 4.26 (2H, t, CH₂OC═O), 5.59 (1H, m, vinylic), 6.11 (1H, bs, vinylic), 6.52 (1H, bs, NH).

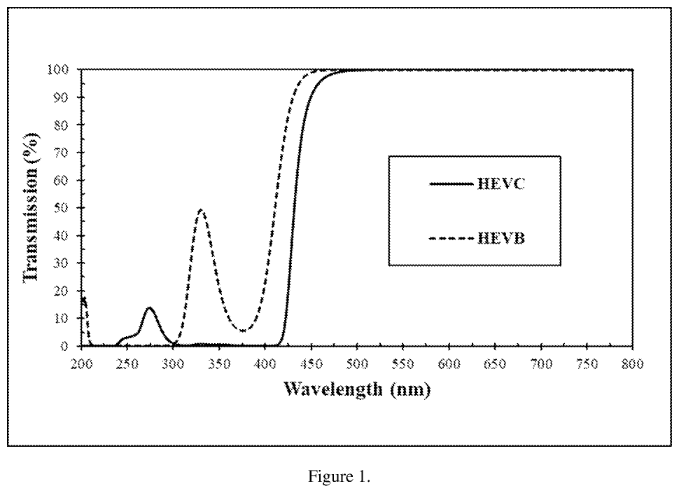

A mixture of 9H-thioxanthene-9-one (2.12 grams, 0.01 mole) and thionyl chloride (5 mL, 8.2 grams, ˜0.07 mole) was refluxed in a 50 mL round bottom flask under a nitrogen atmosphere with constant stirring. After two hours, the red solution was evaporated to dryness ensuring that all unreacted thionyl chloride was removed from the system. 2-(2-Cyanoacetamido)ethyl methacrylate (2.3 grams, 0.0117 mole, ˜1.17 eq.) and 15 mL of dichloromethane were added, and the resulting reaction mixture was heated to reflux under a nitrogen blanket. The reaction was monitored by thin layer chromatography. After two hours, no changes were observed in the chromatogram, so the reactive mixture was allowed to cool down to room temperature. 2-(2-cyano-2-(9H-thioxanthen-9-ylidene)acetamido)ethyl methacrylate (HEVB) was isolated as yellow crystals (3.2 grams, 82% yield) after passing through a short silica gel column (CH₂Cl₂, followed by 8 weight % EtOAc in CH₂Cl₂). ¹H NMR (500 MHz, CDCl₃) δ 1.84 (3H, s, CH₃), 3.47 (2H, m, CH₂NH), 4.01 (2H, t, CH₂OC═O), 5.55 (1H, m, vinylic), 5.91 (1H, bs, NH), 5.98 (1H, bs, vinylic), 7.24 (1H, t, Ar—H), 7.31 (1H, t, Ar—H), 7.39 (2H, m, Ar—H), 7.49 (1H, d, Ar—H), 7.55 (1H, m, Ar—H), 7.61 (1H, d, Ar—H), 8.04 (1H, m, Ar—H). The UV-VIS transmission spectrum of HEVB in 0.2 mM methanol is shown in FIG. 1.

### Preparation 2: Synthesis of 3-((9-(dicyanomethylene)-9H-xanthen-3-yl)oxy)propyl methacrylate (HEVC) as shown in Scheme 2

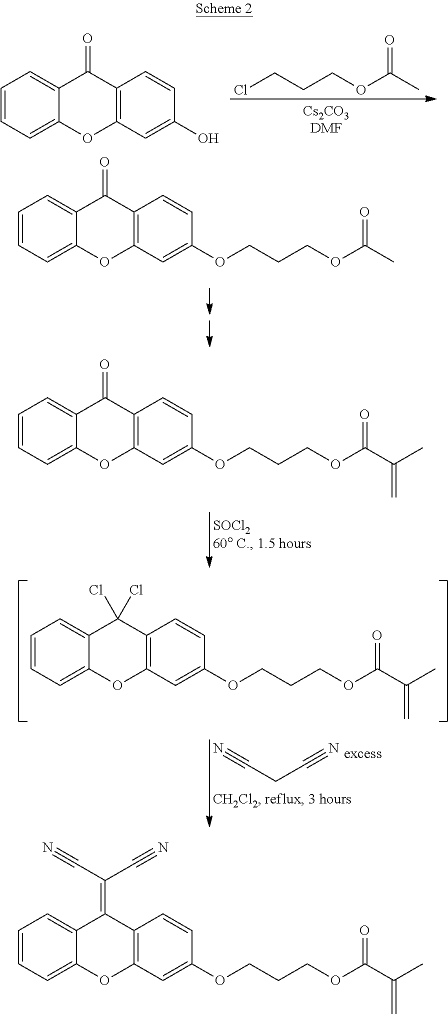

### Synthesis of 3-((9-oxo-9H-xanthen-3-yl)oxy)propyl acetate

A suspension of 3-hydroxy-9H-xanthen-9-one (42.4 grams, 0.2 mole), 70.0 grams Cs₂CO₃ (0.2 mole), and sodium iodide (cat. 200 milligrams) were dried under vacuum in a 500 mL round bottom flask containing a magnetic stirring bar. Anhydrous DMSO (250 mL) was added followed by 2-chloroethyl methacrylate (30.0 grams, 0.2 mole). The reaction mixture was heated overnight at 70° C. Monitoring by TLC indicated complete consumption of the hydroxyxanthenone along with the formation of a less polar derivative. The reaction mixture was cooled to room temperature and slowly poured into dilute aqueous hydrochloric acid with constant stirring. After stirring for thirty minutes, the off-white solids were isolated by vacuum filtration using a fritted glass funnel. The filter cake was washed with deionized water, followed by two washes with 200 mL of hexanes. The 3-((9-oxo-9H-xanthen-3-yl)oxy)propyl acetate was vacuum dried at 60° C. to constant weight.

### Synthesis of 3-((9-oxo-9H-xanthen-3-yl)oxy)propyl alcohol

27 grams of 3-((9-oxo-9H-xanthen-3-yl)oxy)propyl acetate was stirred in about 700 mL of methanol at room temperature, during which 20 mL of 10 N aqueous sodium hydroxide solution was added to the mixture, followed by about 30 mL of deionized water. Monitoring by TLC indicated that the hydrolysis reaction was complete within a few minutes. The mixture was slowly acidified by addition of dilute aqueous hydrochloride acid, after which 150 mL of deionized water was added while constantly stirring the system. The 3-((9-oxo-9H-xanthen-3-yl)oxy)propyl alcohol was isolated by vacuum filtration using a fritted glass funnel, washed with additional amounts of water, and finally dried in a vacuum oven at 60° C.

### Synthesis of 3-((9-oxo-9H-xanthen-3-yl)oxy)propyl methacrylate

25 grams of 3-((9-oxo-9H-xanthen-3-yl)oxy)propanol and 15 mL (10.89 grams) of triethylamine were stirred in 300 mL of anhydrous acetonitrile in a three neck, one liter round bottom flask equipped with a magnetic stirring bar and a reflux condenser. Methacryloyl chloride (9.9 grams) was added to the flask in a dropwise fashion, and mixture was stirred for an hour. The volatile components were evaporated under reduced pressure, and the resulting solids were washed and filtered over a fritted glass funnel and rinsed with deionized water. The residue was washed further with dilute aqueous hydrochloric acid, followed by additional washes with deionized water and finally washed with hexanes. The 3-((9-oxo-9H-xanthen-3-yl)oxy)propyl methacrylate was then dried in a rotary evaporator with bath temperature maintained below 20° C.

### Synthesis of 3-((9-(dicyanomethylene)-9H-xanthen-3-yl)oxy)propyl methacrylate (HEVC)

6.76 grams of 3-((9-oxo-9H-xanthen-3-yl)oxy)propyl methacrylate and 15 mL of thionyl chloride were heated for 2 hours at 65° C. (mantle temperature) in a round bottom flask equipped with a magnetic stirring bar and reflux condenser. The mixture was cooled to room temperature, and the excess thionyl chloride was evaporated under reduced pressure with the bath temperature maintained below 20° C. 3.96 grams of malononitrile was added to the flask, followed by 25 mL of anhydrous dichloromethane, and the mixture was stirred and heated at a gentle reflux for two hours. The mixture was cooled to room temperature and then flushed through a short silica gel plug eluting with methylene chloride. Volatile components were evaporated under reduced pressure with the temperature maintained below 20° C., after which the solids were suspended in cold methanol (100 mL) and stirred for 20 minutes. The crude product was isolated by vacuum filtration and the filter cake washed with additional cold methanol. 3-((9-(dicyanomethylene)-9H-xanthen-3-yl)oxy)propyl methacrylate was further purified by passing through a silica gel column eluting with methylene chloride. ¹H NMR (500 MHz, CDCl₃) δ 1.95 (3H, CH₃), 2.25 (2H, m, CH₂), 4.20 (2H, t, CH₂ benzylic), 4.37 (2H, t, CH₂O ester), 5.59 (1H, m, vinylic), 6.12 (1H, m, vinylic), 6.90 (1H, d, Ar—H), 6.97 (1H, dd, Ar—H), 7.40 (1H, ddd, Ar—H), 7.45 (1H, dd, Ar—H), 7.68 (1H, ddd, Ar—H), 8.50 (1H, d, Ar—H), 8.57 (1H, dd, Ar—H). The UV-VIS transmission spectrum of HEVC in 0.2 mM methanol is shown in FIG. 1.

### Preparation 3: Synthesis of Cyclohexylmethyl Acrylate (CHMA)

Cyclohexyl methanol (25.0 grams, 219.0 mmol) and triethyl amine (33.46 grams, 330.7 mmol) were dissolved in dichloromethane (450 mL) and cooled to about 0° C. using an ice bath. Acryloyl chloride (29.74 grams, 328.5 mmol) was added over a period of 20 minutes while maintaining a constant temperature of about 0° C. After the addition was complete, the reaction mixture was stirred at 0° C. for 30 minutes followed by stirring at ambient temperature overnight. Thin layer chromatography was used to monitor the progress of the reaction. When the reaction was complete, triethyl ammonium chloride was filtered off, dissolved in deionized water (200 mL) and extracted with dichloromethane (3×50 mL). The combined filtrate and organic extracts were washed with water (2×50 mL), brine (25 mL), dried over anhydrous Na₂SO₄, vacuum filtered, and concentrated by rotary evaporation. The crude product was then passed through a short plug of silica gel, eluting with 10% ethyl acetate in n-hexanes, to afford the desired product CHMA as a clear oil (98% yield). ¹H-NMR (500 MHz, CDCl₃): δ 6.39 (1H, dd, J=1.0, 17.0 Hz), 6.12 (1H, dd, J=10.0, 17.0 Hz), 5.81 (1H, dd, J=1.5, 10.0 Hz), 3.97 (2H, d, J=6.0 Hz), 1.76-1.62 (6H, m), 1.31-1.15 (3H, m), 0.95-1.01 (2H, m).

Synthesis of 2-Cyclohexylethyl Acrylate (CHEA): 2-Cyclohexylethyl acrylate was prepared by the same general procedure except that 2-cyclohexyl ethanol was used instead of cyclohexyl methanol (99% yield). ¹H-NMR (500 MHz, CDCl₃): δ 6.38 (1H, dd, J=1.1, 17.2 Hz), 6.11 (1H, dd, J=10.1, 17.2 Hz), 5.80 (1H, dd, J=1.4, 10.1 Hz), 4.18 (2H, t, J=7.0 Hz), 1.74-1.62 (5H, m), 1.58-1.54 (2H, m), 1.39-1.36 (1H, m), 1.27-1.13 (3H, m), 0.97-0.90 (2H, m).

Synthesis of 3-Cyclohexylpropyl Acrylate (CHPA): 3-Cyclohexylpropyl acrylate was prepared by the same general procedure except that 3-cyclohexyl propanol was used instead of cyclohexyl methanol (99% yield). ¹H-NMR (500 MHz, CDCl₃): δ 6.40 (1H, dd, J=1.0, 17.1 Hz), 6.11 (1H, dd, J=10.0, 17.1 Hz), 5.81 (1H, dd, J=1.5, 10.0 Hz), 4.13 (2H, t, J=7.1 Hz), 1.71-1.64 (7H, m), 1.25-1.20 (6H, m), 0.91-0.88 (2H, m).

### Preparation 4: Synthesis N-(2-aminoethyl)pentanamide as shown in Scheme 3

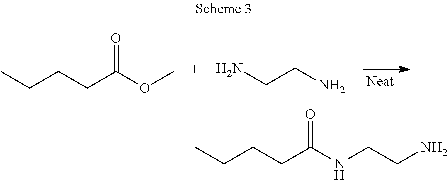

19.7 Grams (0.17 mol) methyl pentanoate and 4.8 grams 1,2-ethylenediamine (0.08 mol) were added to a 100 mL 3-neck round bottom flask charged with a stir bar and outfitted with a reflux condenser containing a nitrogen gas inlet. The reaction mixture was heated to 80° C. for two days. The reaction was monitored by TLC (20% (v/v) MeOH/EtOAc). The volatile components were removed under reduced pressure to yield a solid. The crude product was purified by passing it through a silica plug using a gradient eluent [0 to 20% (v/v) MeOH:EtOAc]. Solvents were removed under reduced pressure to yield the desired mono-acylated product as a white solid. ¹H NMR (CDCl₃, 500 MHz): δ 0.91 (3H, t, J=5.0 Hz, CH₃(CH₂)₃CO), 1.33 (2H, sext, J=5.0 Hz, CH₃CH₂CH₂CH₂CO), 1.59 (2H, qu, J=5.0 Hz, CH₃CH₂CH₂CH₂CO), 1.64 (2H, br s, CH₃CH₂CH₂CH₂CO), 2.17 (2H, t, J=7.5 Hz, RNHCH₂CH₂NH₂), 3.38 (2H, d, J=5.5 Hz, RNHCH₂CH₂NH₂).

### Example 1

### Synthesis of N-Alkyl-2-Hydroxyacetamides as Shown Generically in Scheme 4

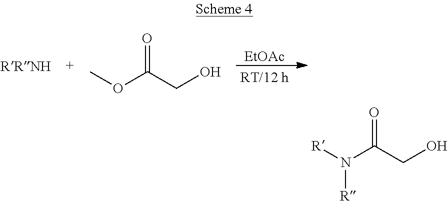

Methyl glycolate (0.35 mol), 120 mL of ethyl acetate, and a primary or secondary amine (0.37 mol) sequentially to a 500 mL round bottom flask charged with a stir bar and equipped with a reflux condenser with nitrogen gas inlet. The reaction mixture was allowed to stir at room temperature (primary amines) or elevated temperatures (secondary amines) while being monitored by TLC (5% (v/v) MeOH/EtOAc). Once complete, the volatile components were removed under reduced pressure to give an oil. 1M hydrochloric acid was added to the oil, and the mixture was extracted 3 times with ethyl acetate. The organics were collected, and the volatiles were removed under reduced pressure to give an oil. For the hydrophilic variations, higher yields were obtained when avoiding an aqueous work up and directly chromatographing on silica (0 to 20% (v/v) MeOH/EtOAc). This general recipe was performed using N-pentylamine, N-cyclohexylmethylamine, N-benzylamine, N-(2-phenylethyl)amine, thiophen-2-ylmethanamine, 3-methoxypropan-1-amine, N-(2-aminoethyl)pentanamide (prophetic), N-octylamine, N-decylamine, (2,2-dimethyl-1,3-dioxolan-4-yl)methanamine and dimethylamine to make the corresponding N-alkyl-2-hydroxyacetamides. The measured or prophetic NMR spectra of the resulting N-alkyl-2-hydroxyacetamides are listed below.

Example 1A: N-pentyl-2-hydroxyacetamide: ¹H NMR (CDCl₃, 500 MHz): δ 0.89 (3H, t, J=7.3 Hz, CH₃CH₂R), 1.27-1.35 (4H, m, CH₃(CH₂)₂CH₂R), 1.53 (2H, quint, J=6.1 Hz, RCH₂CH₂NHR), 3.27-3.31 (2H, m, RCH₂NHR), 4.07 (2H, br s, RCH₂OH), 6.60 (1H, br s, RCH₂NHR).

Example 1B: N-(cyclohexylmethyl)-2-hydroxyacetamide: ¹H NMR (CDCl₃, 500 MHz): δ 0.83-0.91 (2H, m, Cy), 1.04-1.21 (3H, m, Cy), 1.36-1.45 (1H, m, Cy), 1.59-1.62 (1H, m, Cy), 1.64-1.67 (4H, m, Cy), 3.04 (2H, dd, J=6.6 Hz, CyCH₂NHR), 3.96 (2H, s, RCH₂OH), 7.03 (1H, br s, CyCH₂NHR).

Example 1C: N-benzyl-2-hydroxyacetamide: ¹H NMR (CDCl₃, 500 MHz): δ 3.28 (1H, br s, RCH2OH), 4.11 (2H, s, PhCH₂NHR), 4.46 (2H, d, J=6.1 Hz, RCH₂OH), 6.29 (1H, br s, PhCH₂NHR), 7.26-7.29 (3H, m, o,p-Ph), 7.31-7.35 (2H, m, m-Ph).

Example 1D: N-phenethyl-2-hydroxyacetamide: ¹H NMR (CDCl₃, 500 MHz): δ 2.83 (2H, dd, J=6.9 Hz, PhCH₂CH₂N), 3.55 (2H, dd, J=6.9 Hz, PhCH₂CH₂N), 4.02 (2H, s, RCH₂OH), 6.67 (1H, br s, CH₂CH₂NHR), 7.19 (2H, d, J=7.9 Hz, o-Ph), 7.21-7.24 (1H, m, p-Ph), 7.28-7.32 (2H, m, m-Ph).

Example 1E: N-(thiophen-2-ylmethyl)-2-hydroxyacetamide: ¹H NMR (CDCl₃, 500 MHz): δ 3.53 (1H, br s, RCH₂OH), 4.08 (2H, s, RCH₂OH), 4.62 (2H, d, J=5.9 Hz, ArCH₂NHR), 6.94 (1H, dd, J=3.4, 5.1 Hz, C-4), 6.96-6.97 (1H, m, C-5), 7.04 (1H, br s, ArCH2NHR), 7.21 (1H, dd, J=3.9, 5.1 Hz, C-6).

Example 1F: 2-hydroxy-N-(3-methoxypropyl)acetamide: ¹H NMR (CDCl₃, 500 MHz): δ 1.80 (2H, dt, J=5.5, 6.5 Hz, OCH₂CH₂CH₂N), 3.34 (3H, s, CH₃OCH₂), 3.42 (2H, t, J=6.5 Hz, OCH₂CH₂CH₂N), 3.48 (2H, t, J=5.5 Hz, OCH₂CH₂CH₂N), 4.07 (2H, d, J=6. Hz, RCH₂OH).

Example 1G: N-(2-(2-hydroxyacetamido)ethyl)pentanamide: ¹H NMR (CDCl₃, 500 MHz): δ 0.93 (3H, t, CH₃(CH₂)₃CO), 1.38 (2H, sext, CH₃CH₂CH₂CH₂CO), 1.53 (2H, qu, CH₃CH₂CH₂CH₂CO), 2.13 (2H, t, CH₃CH₂CH₂CH₂CO), 3.66 (4H, s, HN(CH₂)₂NH), 4.42 (2H, s, RCH₂OH) [prophetic via ChemDraw software].

Example 1H: 2-hydroxy-N-octylacetamide: ¹H NMR (CDCl₃, 500 MHz): δ 0.83 (3H, t, J=6.5 Hz, CH₃CH₂R), 1.22-1.28 (10H, m, CH₃(CH₂)₅CH₂R), 1.47 (2H, br qu, CH₃(CH₂)₅CH₂R), 3.21 (2H, dd, J=6.5, 7.5 Hz, RCH₂NHR), 3.97 (2H, s, RCH₂OH), 4.96 (1H, br s, RCH₂OH), 6.94 (1H, br s, RCH₂NHR).

Example 1I: 2-hydroxy-N-decylacetamide: ¹H NMR (CDCl₃, 500 MHz): δ 0.87 (3H, t, J=7.5 Hz, CH₃CH₂R), 1.25-1.29 (14H, m, CH₃(CH₂)₇CH₂R), 1.50 (2H, br qu, CH₃(CH₂)₇CH₂R), 3.26 (2H, dd, J=5, 10 Hz, RCH₂NHR), 3.64 (1H, br s, RCH₂OH), 4.05 (2H, s, RCH₂OH), 6.65 (1H, br s, RCH₂NHR).

Example 1J: N-((2,2-dimethyl-1,3-dioxolan-4-yl)methyl)-2-hydroxyacetamide: ¹H NMR (CDCl₃, 500 MHz): δ 1.34 (3H, s, OC(CH₃)₂O), 1.43 (3H, s, OC(CH₃)₂O), 3.35-3.40 (1H, m), 3.55-3.59 (1H, m), 3.62-3.66 (1H, m), 4.03-4.06 (1H, m), 4.11-4.13 (2H, m), 4.24-4.28 (1H, m).

Example 1K: 2-hydroxy-N,N-dimethylacetamide: ¹H NMR (d₆-DMSO, 500 MHz): δ 2.98 (6H, s, N—CH₃), 4.42 (2H, s, CH₂), 4.91 (1H, s, OH) [prophetic via ChemDraw software].

### Example 2

Synthesis of 2-oxo-2-(alkylamino)ethyl acrylates or 2-oxo-2-(arylamino)ethyl acrylate as shown generically in Scheme 5 starting from N-alkyl-2-hydroxyacetamides (R″=hydrogen or methyl):

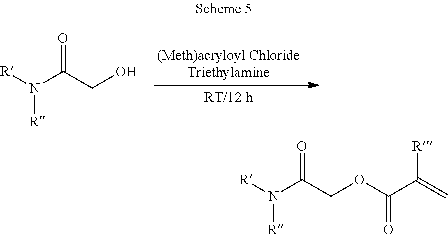

N-Alkyl-2-hydroxyacetamide or N-alkyl (R′)—N-alkyl (R″)-2-hydroxyacetamide (0.30 mol), 250 mL of dichloromethane, and triethylamine (0.36 mol) were sequentially added to a heat gun-dried one liter three neck round bottom charged with a magnetic stirring bar and equipped with an addition funnel and reflux condenser with a nitrogen gas inlet. (Meth)acryloyl chloride (0.36 mol, R′″=H or methyl) was charged into the addition funnel and slowly added to the stirred reaction mixture over about one hour at room temperature. Reaction progress was monitored by TLC (5% (v/v) MeOH/EtOAc). Once completed, about 20 mL of MeOH was charged to the addition funnel and slowly added to quench the excess acid chloride. The reaction mixture was allowed to stir overnight (approximately 12 hours) at room temperature. Volatile components were then removed under reduced pressure to yield an oil. The oil was further purified by passing it through a short plug of silica gel, eluting with 20% (v/v) EtOAc/hexane. This general recipe was performed using N-pentyl-2-hydroxyacetamide, N-(cyclohexylmethyl)-2-hydroxyacetamide, N-benzyl-2-hydroxyacetamide, N-phenethyl-2-hydroxyacetamide, and N-(thiophen-2-ylmethyl)-2-hydroxyacetamide, 2-hydroxy-N-(3-methoxypropyl)acetamide, N-(2-(2-hydroxyacetamido)ethyl)pentanamide (prophetic), 2-hydroxy-N-octylacetamide, 2-hydroxy-N-decylacetamide, and 2-hydroxy-N,N-dimethylacetamide to make the corresponding amide acrylates. The measured and prophetic NMR spectra of the resulting amide acrylates are listed below.

Example 2A: 2-oxo-2-(pentylamino)ethyl acrylate (OPEA): ¹H NMR (CDCl₃, 500 MHz): δ 0.89 (3H, t, J=6.8 Hz, CH₃CH₂R), 1.26-1.37 (4H, m, CH₃(CH₂)₂CH₂R), 1.53 (2H, quint, J=7.8 Hz, RCH₂CH₂NHR), 3.30 (2H, dd, J=7.8 Hz, RCH₂NHR), 4.64 (2H, s, RCH₂OH), 5.95 (1H, dd, J=1.0, 10.4 Hz, vinylic), 6.10 (1H, br s, PhCH₂NHR), 6.20 (1H, dd, J=10.4, 17.2 Hz, vinylic), 6.51 (1H, dd, J=1.0, 17.2 Hz, vinylic).

Example 2B: 2-((cyclohexylmethyl)amino)-2-oxoethyl acrylate (CHOPE): ¹H NMR (CDCl₃, 500 MHz): δ 0.89-0.95 (2H, m, Cy), 1.10-1.25 (3H, m, Cy), 1.48 (1H, br s, Cy), 1.64-1.74 (5H, m, Cy), 3.15 (2H, dd, J=5.8 Hz, CyCH₂NHR), 4.65 (2H, s, RCH₂OH), 5.96 (1H, dd, J=1.3, 11.6 Hz, vinylic), 6.15 (1H, br s, PhCH₂NHR), 6.21 (1H, dd, J=10.4, 17.2 Hz, vinylic), 6.51 (1H, dd, J=1.3, 17.2 Hz, vinylic).

Example 2C: 2-(benzylamino)-2-oxoethyl acrylate (BZOEA): ¹H NMR (CDCl₃, 500 MHz): δ 4.46 (2H, d, J=5.7 Hz, PhCH₂N(CH₃)R), 4.65 (2H, s, RCH₂OH), 5.09 (1H, dd, J=1.1, 10.3 Hz, vinylic), 6.16 (1H, dd, J=10.3, 17.6 Hz, vinylic), 6.47 (1H, dd, J=1.1, 17.6 Hz, vinylic), 6.71 (1H, br s, PhCH₂NHR), 7.24-7.28 (3H, m, o,p-Ph), 7.31-7.34 (2H, m, m-Ph).

Example 2D: 2-oxo-2-(phenethylamino)ethyl acrylate (OPEEA): ¹H NMR (CDCl₃, 500 MHz): δ 2.84 (2H, dd, J=7.0 Hz, PhCH₂CH₂N), 3.58 (2H, dd, J=6.4 Hz, PhCH₂CH₂N), 4.62 (2H, s, RCH₂OH), 5.91 (1H, dd, J=1.0, 10.4 Hz, vinylic), 6.11 (1H, dd, J=10.5, 17.8 Hz, vinylic and 1H, br s, PhCH₂NHR), 6.41 (1H, dd, J=1.0, 17.8 Hz, vinylic), 7.17-7.19 (1H, m, o-Ph), 7.22-7.25 (1H, m, p-Ph), 7.29-7.32 (2H, m, m-Ph).

Example 2E: 2-oxo-2-((thiophen-2-ylmethyl)amino)ethyl acrylate (OTPEA): ¹H NMR (CDCl₃, 500 MHz): δ 4.61 (2H, d, J=6.2 Hz, ArCH₂NHR), 4.62 (2H, s, RCH₂OH), 5.89 (1H, dd, J=1.0, 10.4 Hz, vinylic), 6.15 (1H, dd, J=10.6, 17.4 Hz, vinylic), 6.45 (1H, dd, J=1.0, 17.4 Hz, vinylic), 6.78 (1H, br s, ArCH₂NHR), 6.92 (1H, dd, J=3.4, 5.2 Hz, C-8), 6.94-6.95 (1H, m, C-7), 7.19 (1H, dd, J=1.5, 5.4 Hz, C-9).

Example 2F: 2-((3-methoxypropyl)amino)-2-oxoethyl acrylate (MOPEA): ¹H NMR (CDCl₃, 500 MHz): δ 1.77 (2H, br qu, OCH₂CH₂CH₂N), 3.32 (3H, br s, CH₃OCH₂), 3.41-3.45 (2H, m, OCH₂CH₂CH₂N), 3.49-3.52 (2H, m, OCH₂CH₂CH₂N), 4.64 (2H, d, J=3.5 Hz, RCH₂OH), 5.94 (1H, br dd, vinylic), 6.16 (1H, br dd, vinylic), 6.49 (1H, br dd, vinylic), 6.88 (1H, br s, CH₂NHR).

Example 2G: 2-oxo-2-((2-pentanamidoethyl)amino)ethyl acrylate (OPAEA): ¹H NMR (CDCl₃, 500 MHz): δ 0.93 (3H, t, CH₃(CH₂)₃CO), 1.38 (2H, sext, CH₃CH₂CH₂CH₂CO), 1.53 (2H, qu, CH₃CH₂CH₂CH₂CO), 2.13 (2H, t, CH₃CH₂CH₂CH₂CO), 3.66 (4H, s, NHCH₂CH₂NH), 4.96 (2H, s, RCH₂OH), 5.83 (1H, dd, vinylic), 6.12 (1H, dd, vinylic), 6.41 (1H, dd, vinylic) [prophetic via ChemDraw software].

Example 2H: 2-(octylamino)-2-oxoethyl acrylate (OAOEA): ¹H NMR (CDCl₃, 500 MHz): δ 0.87 (3H, t, J=3.5 Hz, CH₃CH₂R), 1.25-1.29 (10H, m, CH₃(CH₂)₅CH₂R), 1.49 (2H, br qu, CH₃(CH₂)₅CH₂R), 3.29 (2H, dd, J=6.5, 7.5 Hz, RCH₂NHR), 4.65 (2H, s, RCH₂OH), 5.95 (1H, dd, vinylic), 6.07 (1H, br s, RCH₂NHR), 6.183 (1H, dd, vinylic), 6.49 (1H, dd, vinylic).

Example 21: 2-(decylamino)-2-oxoethyl acrylate (DAOEA): ¹H NMR (CDCl₃, 500 MHz): δ 0.84 (3H, t, J=3.5 Hz, CH₃CH₂R), 1.22-1.26 (14H, m, CH₃(CH₂)₇CH₂R), 1.49 (2H, br qu, CH₃(CH₂)₇CH₂R), 3.26 (2H, dd, J=5.0, 9.0 Hz, RCH₂NHR), 4.61 (2H, s, RCH₂OH), 5.91 (1H, dd, J=1.0, 10.0 Hz, vinylic), 6.15-6.21 (1H, br s, RCH₂NHR and 1H, dd, J=7.0, 10.0 Hz, vinylic), 6.46 (1H, dd, J=1.0, 7.0 Hz, vinylic).

Example 2J: 2-(((2,2-dimethyl-1,3-dioxolan-4-yl)methyl)amino)-2-oxoethyl methacrylate (DOOEMA): ¹H NMR (CDCl₃, 500 MHz): δ 1.21 (6H, s, OC(CH₃)₂O), 2.01 (3H, s, methyl), 3.25-3.50 (2H, m, NCH₂CHOCH₂O), 3.62-3.87 (2H, m, NCH₂CHOCH₂O), 4.54 (1H, m, NCH₂CHOCH₂O), 4.96 (2H, s, OCH₂C(O)N), 6.40 (1H, d, vinylic), 6.48 (1H, d, vinylic) [prophetic via ChemDraw software].

Example 2K: 2-(dimethylamino)-2-oxoethyl methacrylate (DMAOEMA): ¹H NMR (CDCl₃, 500 MHz): δ 1.99 (3H, s, CH₂=CCH₃), 2.97 (3H, s, NCH₃), 2.99 (3H, s, NCH₃), 4.79 (2H, s, OCH₂C(O)N), 5.64 (1H, s, vinylic), 6.22 (1H, s, vinylic).

Example 2L: Synthesis 2-((2,3-dihydroxypropyl)amino-2-oxoethyl methacrylate (DHOEMA) (prophetic), as shown in Scheme 6:

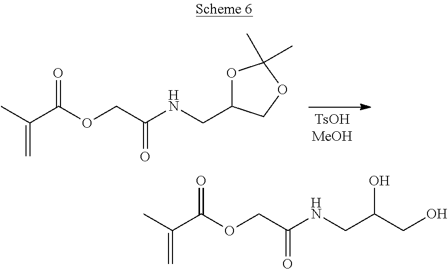

2-(((2,2-dimethyl-1,3-dioxolan-4-yl)methyl)amino)-2-oxoethyl methacrylate and tosylic acid are added to a methanol-dioxane solution in a 100 mL 3-neck round bottom flask charged with a stir bar and outfitted with a reflux condenser containing a nitrogen gas inlet. The reaction is monitored by TLC. Volatile components are removed under reduced pressure. The crude product is redissolved in a suitable solvent and passed over a silica plug. The purified fraction is collected, and the volatile components are removed by rotary evaporation, thereby yielding the desired compound. ¹H NMR (CDCl₃, 500 MHz): δ 2.01 (3H, s, methyl), 3.25-3.52 (2H, m, NCH₂CHOCH₂O), 3.62-3.87 (2H, m, NCH₂CHOCH₂O), 4.22 (1H, m, NCH₂CHOCH₂O), 4.96 (2H, s, OCH₂C(O)N), 6.40 (1H, d, vinylic), 6.48 (1H, d, vinylic) [prophetic via ChemDraw software].

### Standard Curing, Demolding, and Extraction Procedures (“SCDEP”)

Unless noted otherwise, all polymer disks were fabricated by the following standardized curing, demolding, and extraction procedures: Under yellow lighting, reactive monomer mixtures (RMM) were degassed using vacuum for at least 7 minutes, back filling the head space with nitrogen gas, and then immediately transferred into a fill box having a nitrogen gas atmosphere with less than 0.1% to 0.5% (v/v) oxygen gas and an internal temperature at ambient temperature. Polymer disks (about 0.75 millimeters in thickness and 12-14 millimeters in diameter) were fabricated using circular plastic molds made of polypropylene. About 250 microliters of RMM were dispensed into the mold assembly, and the assembly transferred into a cure box held at a temperature between 43° C. and 47° C. and then cured from the top and bottom for a total of ninety minutes using 435 nm LED lights on both sides with the following intensity profile: 20 minutes at 5 mW/cm² (2.5 mW/cm² top and 2.5 mW/cm² bottom), 20 minutes at 10 mW/cm² (5 mW/cm² top and 5 mW/cm² bottom), 20 minutes at 20 mW/cm² (10 mW/cm² top and 10 mW/cm² bottom) and 30 minutes at 30 mW/cm² (15 mW/cm² top and 15 mW/cm² bottom). The cured assemblies were manually demolded. The polymer disks were all transparent and exhibited low levels of surface tackiness. Each disk was extracted with 2-propanol and subsequently dried using the following steps: (a) one disk was transferred into a glass jar with 20 mL of 2-propanol and shaken at 115 rpm for 24 hours at 50° C. using an incubator/shaker, (b) the 2-propanol was completely decanted, replaced with a fresh 20 mL aliquot of 2-propanol, and shaken at 115 rpm for 1.5 hours at 50° C., (c) step (b) was repeated two more times, (d) after the final solvent decant, the polymer disk was allowed to air dry at room temperature overnight, and then (e) the disk was placed in a vacuum oven at 60-65° C. for seven days (less than one inch of mercury).

### Alternate Acetonitrile Extraction Procedure (“AAEP”)

In some cases, the polymer disks were placed in circular PEEK extraction vehicles and extracted with acetonitrile instead of 2-propanol. Each vehicle is about 635 millimeters in diameter and 8 millimeters thick and consists of five circular recessed extraction houses about 20 millimeters in diameter and 5 millimeters depth. The vehicles are designed for optimal solvent flow through/exchange and are stackable. Stacking is accomplished via a circular hole in the center of the vehicle, about 10 millimeters in diameter, and a threaded PEEK rod of suitable diameter and length. Polymer disks were individually placed in the extraction houses of the extraction vehicle. The vehicles were stacked, and a blank extraction vehicle (containing no polymer disks) was used to cap the top of the stack. The stack was placed in a glass jar and acetonitrile was added to attain a polymer disk to volume of acetonitrile ratio of 1 to 14 mL. The jar was placed on an orbital shaker at ambient conditions and shaken at 115 rpm overnight (15-17 hours). The acetonitrile was completely decanted, and a fresh aliquot of acetonitrile was added to attain a polymer disk to volume of acetonitrile ratio of 1 to 14 mL. The glass jar was shaken at 115 rpm for 4 hours, after which a final acetonitrile exchange was done for an additional 4 hours of extraction at 115 rpm. The stack was subsequently removed from the glass jar, thoroughly drained, and allowed to air dry at ambient conditions for 24 hours. For the final drying step, the stack was placed in a vacuum oven at 60° C. to 65° C. for four days (less than one inch of mercury).

### Examples 3-6

Polymer disks were fabricated using the standard curing, demolding, and extraction procedures (SCDEP) using the RMM listed in Table 1. For each example, the refractive index, Abbe number, and water content were determined on the dried disks. The average refractive indexes, Abbe numbers, and water contents are also listed in Table 1. Standard deviations are reported within the parentheses.

TABLE 1
Formulations and Physical Properties

| Components | | | | |
|---|---|---|---|---|
| (weight %) | Ex. 3 | Ex. 4 | Ex. 5 | Ex. 6 |
| EGDCA | 72.35 | 72.35 | 72.35 | 72.35 |
| PEG-OH 360 | 12 | 6 | 12 | 12 |
| OPEA | 4 | 10 | 0 | 0 |
| CHOPE | 0 | 0 | 4 | 0 |
| OPEEA | 0 | 0 | 0 | 4 |
| NHA | 7.5 | 7.5 | 7.5 | 7.5 |
| UVB | 0.7 | 0.7 | 0.7 | 0.7 |
| TCDA | 3 | 3 | 3 | 3 |
| Omnirad 819 | 0.45 | 0.45 | 0.45 | 0.45 |
| Σ Components | 100 | 100 | 100 | 100 |

| Properties | Ex. 3 | Ex. 4 | Ex. 5 | Ex. 6 |
|---|---|---|---|---|
| RI (25) | 1.522288 | 1.523085 | 1.523949 | 1.525283 |
| | (0.004415) | (0.004639) | (0.004343) | (0.004627) |
| Abbe # (25) | 54 | 52 | 55 | 52 |
| | (1) | (0) | (1) | (0) |
| WC (wt. %) | 2.297 | 2.023 | 2.005 | 2.181 |
| | (0.367) | (0.169) | (0.021) | (0.368) |

Examples 3-6 exhibited both high refractive indices (greater than 1.50) and high Abbe numbers (greater than 50) making these materials most suitable for ophthalmic devices such as intraocular lenses. Examples 3-6 also exhibited low water contents which can provide dimensional stability for implantable ophthalmic devices.

### Examples 7-11

Polymer disks were fabricated using the standard curing, demolding, and extraction procedures (SCDEP) using the RMM listed in Table 2. For each example, the refractive index, Abbe number, and water content were determined on the dried disks. The average refractive indexes, Abbe numbers, and water contents are also listed in Table 2. Standard deviations are reported within the parentheses.

TABLE 2
Formulations and Physical Properties

| Components | | | | | |
|---|---|---|---|---|---|
| (weight %) | Ex. 7 | Ex. 8 | Ex. 9 | Ex. 10 | Ex. 11 |
| EGDCA | 70 | 71.95 | 55.8 | 57.05 | 56.95 |
| PEG-OH 360 | 8 | 8 | 0 | 0 | 0 |
| PEPEA | 0 | 0 | 11 | 11.7 | 11.7 |
| BCEA | 9 | 9 | 0 | 0 | 0 |
| NBA | 6.3 | 6.3 | 14 | 14 | 14 |
| HEMA | 0 | 0 | 15 | 15 | 15 |
| HEVB | 2.25 | 0 | 2.25 | 0 | 0 |
| HEVC | 0 | 0.3 | 0 | 0.3 | 0.4 |
| TCDA | 3 | 3 | 1.5 | 1.5 | 1.5 |
| EGDMA | 1 | 1 | 0 | 0 | 0 |
| Omnirad 819 | 0.45 | 0.45 | 0.45 | 0.45 | 0.45 |
| Σ Components | 100 | 100 | 100 | 100 | 100 |

| Properties | Ex. 7 | Ex. 8 | Ex. 9 | Ex. 10 | Ex. 11 |
|---|---|---|---|---|---|
| RI (25) | 1.524561 | 1.521600 | 1.521078 | 1.518115 | 1.518200 |
| | (0.000189) | (0.000144) | (0.000207) | (0.000200) | (0.000329) |
| Abbe # (25) | 49.85 | 53.62 | 48.80 | 51.53 | 51.02 |
| | (0.63) | (0.62) | (0.38) | (0.31) | (0.60) |
| WC (wt. %) | 1.43 | Not | 2.34 | Not | Not |
| | (0.42) | Measured | (0.28) | Measured | Measured |

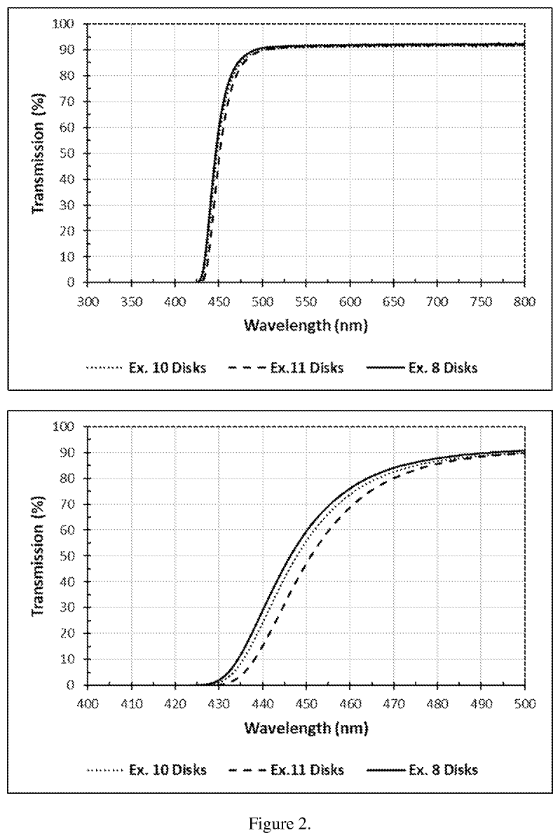

By replacing HEVB with HEVC at a lower concentration in examples 7 and 8, the refractive index was maintained at about 1.52 but the Abbe number increased from 49.85 to 53.62. Similarly, by replacing HEVB with HEVC at lower concentrations in examples 9-11, the refractive index was maintained at about 1.52 but the Abbe number increased from 49.8 to 51.0 or higher. As a result, lenses with low concentrations of HEVC exhibited both high refractive indices (greater than 1.50) and high Abbe numbers (greater than 50) making these materials most suitable for ophthalmic devices such as intraocular lenses. Examples 7 and 9 also exhibited low water contents which can provide dimensional stability for implantable ophthalmic devices. The UV-VIS transmission spectra of Ex. 8 disks, Ex. 10 disks, and Ex. 11 disks are shown in FIG. 2. These samples absorbed essentially all light having wavelengths between 300 nanometers and 430 nanometers.

### Examples 12-19

Polymer disks were fabricated using the standard curing, demolding, and extraction procedures (SCDEP) using the RMM listed in Tables 3A and 3B. For each example, the refractive index, Abbe number, and water content were determined on the dried disks. The average refractive indexes, Abbe numbers, and water contents are listed in Tables 3A and 3B. Standard deviations are reported within the parentheses.

TABLE 3A
Formulations and Physical Properties

| Components | | | | |
|---|---|---|---|---|
| (weight %) | Ex. 12 | Ex. 13 | Ex. 14 | Ex. 15 |
| EGDCA | 66 | 66 | 68.8 | 70.75 |
| OPEA | 15 | 15 | 10 | 10 |
| NHA | 14.8 | 16.75 | 9.5 | 9.5 |
| HBA | 0 | 0 | 6 | 6 |
| HEVB | 2.25 | 0 | 2.25 | 0 |
| HEVC | 0 | 0.3 | 0 | 0.3 |
| TCDA | 1.5 | 1.5 | 3 | 3 |
| Omnirad 819 | 0.45 | 0.45 | 0.45 | 0.45 |
| Σ Components | 100 | 100 | 100 | 100 |

| Properties | Ex. 12 | Ex. 13 | Ex. 14 | Ex. 15 |
|---|---|---|---|---|
| RI (25) | 1.519415 | 1.514799 | 1.523404 | 1.519686 |
| | (0.000184) | (0.000078) | (0.000157) | (0.000067) |
| Abbe # (25) | 49.55 | 51.64 | 49.27 | 51.93 |
| | (0.59) | (0.41) | (0.48) | (0.31) |
| WC (wt. %) | 1.09 | 1.14 | 1.09 | 1.10 |
| | (0.17) | (0.08) | (0.15) | (0.16) |
| T_{g} (° C.) | 11.0 | 6.8 | — | — |
| Tan δₘₐₓ (° C.) | 25.7 | 20.5 | — | 27.2 |
| E′ (MPa) | 48.5 | 12.8 | — | 97 |
| | (0.8) | (0.4) | | (2) |

TABLE 3B
Formulations and Physical Properties

| Components | | | | |
|---|---|---|---|---|
| (weight %) | Ex. 16 | Ex. 17 | Ex. 18 | Ex.19 |
| EGDCA | 68.75 | 68.75 | 67.24 | 67.75 |
| OPEA | 6 | 0 | 9.85 | 4 |
| NHA | 9.5 | 9.5 | 9.36 | 9.5 |
| HBA | 12 | 18 | 9.85 | 15 |
| HEVB | 0 | 0 | 0 | 0 |
| HEVC | 0.3 | 0.3 | 0.3 | 0.3 |
| TCDA | 3 | 3 | 2.96 | 3 |
| Omnirad 819 | 0.45 | 0.45 | 0.45 | 0.45 |
| Σ Components | 100 | 100 | 100 | 100 |

| Properties | Ex. 16 | Ex. 17 | Ex. 18 | Ex.19 |
|---|---|---|---|---|
| RI (25) | 1.519121 | 1.519282 | 1.519033 | 1.518776 |
| | (0.000137) | (0.000150) | (0.000045) | (0.000160) |
| Abbe # (25) | 52.19 | 52.58 | 52.77 | 52.95 |
| | (0.44) | (0.32) | (0.14) | (0.24) |
| WC (wt. %) | 1.31 | 1.83 | 1.27 | 1.57 |
| | (0.07) | (0.51) | (0.08) | (0.13) |
| T_{g} (° C.) | — | — | — | — |
| Tan δₘₐₓ (° C.) | 24.5 | 22.0 | 25.1 | 25.5 |
| E′ (MPa) | 35.5 | 17.9 | 51.4 | 66 |
| | (0.4) | (0.1) | (0.5) | (1) |

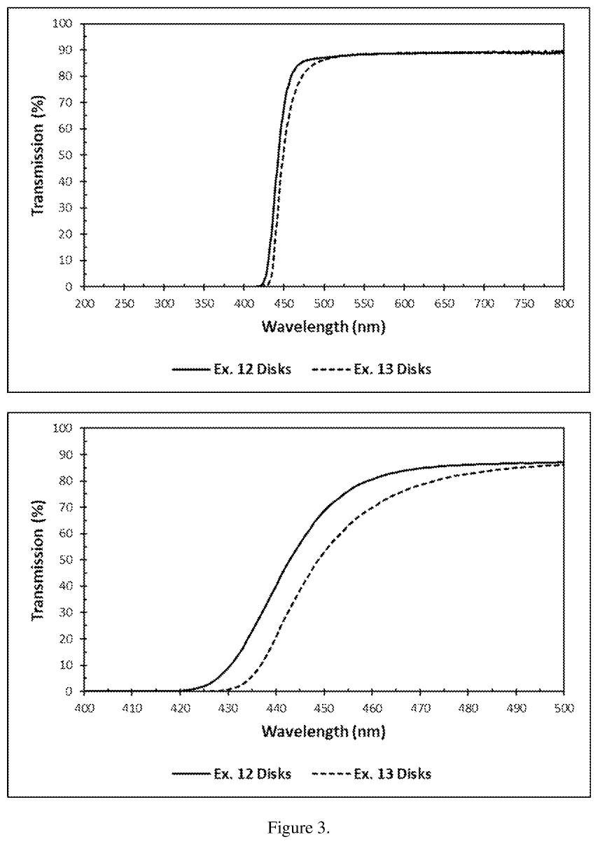

By replacing HEVB with HEVC at a lower concentration in examples 12 and 13, the refractive index was maintained at about 1.51 but the Abbe number increased from 49.55 to 51.64. Similarly, by replacing HEVB with HEVC at lower concentrations in examples 14-19, the refractive index was maintained at about 1.52 but the Abbe number increased from 49.17 to 51.93 or higher. As a result, disks with low concentrations of HEVC exhibited both high refractive indices (greater than 1.50) and high Abbe numbers (greater than 50) making these materials most suitable for ophthalmic devices such as intraocular lenses. Examples 12-19 also exhibited low water contents which can provide dimensional stability for implantable ophthalmic devices. The UV-VIS transmission spectra of Ex. 12 disks and Ex. 13 disks are shown in FIG. 3. Example 12 essentially absorbed all light having wavelengths between 200 nanometers and 420 nanometers, while Example 13 lenses essentially absorbed all light having wavelengths between 200 nanometers and 430 nanometers. The glass transition temperatures, tan deltas and storage moduli are also listed in Tables 3A and 3B.

### Examples 20-25

Polymer disks were fabricated using the standard curing, demolding, and extraction procedures (SCDEP) using the RMM listed in Table 4. For each example, the refractive index, Abbe number, and water content were determined on the dried disks. The average refractive indexes, Abbe numbers, water contents, tan deltas, and storage moduli are listed in Table 4. Standard deviations are reported within the parentheses.

TABLE 4
Formulations and Physical Properties

| | Ex. 20 | Ex. 121 | Ex. 22 | Ex. 23 | Ex. 24 | Ex. 25 |
|---|---|---|---|---|---|---|
| Components | | | | | | |
| (weight %) | | | | | | |
| EGDCA | 70 | 71.95 | 68 | 69.95 | 65 | 67.95 |
| mPEG 300 | 8 | 8 | 0 | 0 | 0 | 0 |
| E2EA | 0 | 0 | 10 | 10 | 0 | 0 |
| BCEA | 9 | 9 | 9 | 9 | 9 | 9 |
| NHA | 7.3 | 7.3 | 7.3 | 7.3 | 11.8 | 10.8 |
| HBA | 0 | 0 | 0 | 0 | 10 | 10 |
| HEVB | 2.25 | 0 | 2.25 | 0 | 2.25 | 0 |
| HEVC | 0 | 0.3 | 0 | 0.3 | 0 | 0.3 |
| TCDA | 3 | 3 | 3 | 3 | 1.5 | 1.5 |
| Omnirad 819 | 0.45 | 0.45 | 0.45 | 0.45 | 0.45 | 0.45 |
| Σ Components | 100 | 100 | 100 | 100 | 100 | 100 |
| Properties | | | | | | |
| RI (25) | 1.522157 | 1.518634 | 1.520831 | 1.517743 | 1.519627 | 1.517227 |
| | (0.000095) | (0.000059) | (0.000120) | (0.000155) | (0.000092) | (0.000108) |
| Abbe # (25) | 50.39 | 53.93 | 48.94 | 52.22 | 49.18 | 52.54 |
| | (0.28) | (0.48) | (0.53) | (0.33) | (0.76) | (0.24) |
| WC (wt. %) | 0.67 | 0.83 | 0.72 | 0.96 | 1.11 | 1.04 |
| | (0.05) | (0.09) | (0.16) | (0.41) | (0.02) | (0.05) |
| Tan δₘₐₓ (° C.) | 23.0 | 20.6 | — | — | 21.4 | 19.9 |
| E′ (MPa) | 22.6 | 14.1 | — | — | 14.8 | 11.0 |
| | (0.1) | (0.03) | | | (0.9) | (0.3) |

Examples 20 and 21 prepared from a reactive monomer mixture containing at least 70 weight percent EGDCA exhibited both high refractive indices (greater than 1.50) and high Abbe numbers (greater than 50) making these materials most suitable for ophthalmic devices such as intraocular lenses. When the reactive monomer mixture contained less than 70 weight percent EGDCA, only Examples 23 and 25 which included HEVC in low concentration exhibited both high refractive indices (greater than 1.50) and high Abbe numbers (greater than 50) making these materials most suitable for ophthalmic devices such as intraocular lenses. Examples 20-25 also exhibited low water contents which can provide dimensional stability for implantable ophthalmic devices.

### Examples 26-32

Polymer disks were fabricated using the standard curing, demolding, and extraction procedures (SCDEP) using the RMM listed in Table 5. For each example, the refractive index, Abbe number, and water content were determined on the dried disks. The average refractive indexes, Abbe numbers, and water contents are listed in Table 5. Standard deviations are reported within the parentheses.

TABLE 5
Formulations and Physical Properties

| | Ex. 26 | Ex. 27 | Ex. 28 | Ex. 29 | Ex. 30 | Ex. 31 | Ex. 32 |
|---|---|---|---|---|---|---|---|
| Components | | | | | | | |
| (weight %) | | | | | | | |
| EGDCA | 65 | 65 | 68.8 | 68.8 | 70.75 | 68.8 | 70.25 |
| mPEG 300 | 0 | 0 | 6 | 0 | 0 | 0 | 0 |
| E2EA | 0 | 0 | 0 | 0 | 0 | 6 | 6 |
| BCEA | 5 | 0 | 0 | 0 | 0 | 0 | 0 |
| OPEA | 12 | 10 | 10 | 10 | 10 | 10 | 10 |
| HEMA | 0 | 6 | 0 | 0 | 0 | 0 | 0 |
| HBA | 0 | 0 | 0 | 6 | 6 | 0 | 0 |
| NHA | 14.3 | 15.3 | 9.5 | 9.5 | 9.5 | 9.5 | 9.5 |
| HEVB | 2.25 | 2.25 | 2.25 | 2.25 | 0 | 2.25 | 0 |
| HEVC | 0 | 0 | 0 | 0 | 0.3 | 0 | 0.3 |
| TCDA | 1 | 1 | 3 | 3 | 3 | 3 | 3 |
| Omnirad 819 | 0.45 | 0.45 | 0.45 | 0.45 | 0.45 | 0.45 | 0.45 |
| Σ Components | 100 | 100 | 100 | 100 | 100 | 100 | 100 |
| Properties | | | | | | | |
| RI (25) | 1.518317 | 1.519058 | 1.521829 | 1.523404 | 1.519686 | 1.521647 | 1.518577 |
| | (0.000145) | (0.000078) | (0.000098) | (0.000016) | (0.000067) | (0.000236) | (0.000096) |
| Abbe # (25) | 49.16 | 49.69 | 49.82 | 49.27 | 51.93 | 49.80 | 52.08 |
| | (0.21) | (0.35) | (0.65) | (0.48) | (0.31) | (0.34) | (0.14) |
| WC (wt. %) | 0.94 | 1.25 | 1.00 | 1.09 | 1.10 | 0.79 | 0.87 |
| | (0.01) | (0.19) | (0.05) | (0.15) | (0.16) | (0.08) | (0.08) |

Examples 30 and 32 prepared from a reactive monomer mixture containing at least 70 weight percent EGDCA and HEVC exhibited both high refractive indices (greater than 1.50) and high Abbe numbers (greater than 50) making these materials most suitable for ophthalmic devices such as intraocular lenses. When the reactive monomer mixture contained less than 70 weight percent EGDCA and HEVB, as used in Examples 26-29 and 31, the samples exhibited high refractive indices of about 1.52 but the Abbe numbers were slightly below 50, but still suitable for ophthalmic devices such as intraocular lenses. Examples 26-32 also exhibited low water contents which can provide dimensional stability for implantable ophthalmic devices.

### Examples 33-38

Polymer disks were fabricated using the standard curing, demolding, and extraction procedures (SCDEP) using the RMM listed in Table 6. For each example, the refractive index, Abbe number, and water content were determined on the dried disks. The average refractive indexes, Abbe numbers, and water contents are listed in Table 6. Standard deviations are reported within the parentheses.

TABLE 6
Formulations and Physical Properties

| | Ex. 33 | Ex. 34 | Ex. 35 | Ex. 36 | Ex. 37 | Ex. 38 |
|---|---|---|---|---|---|---|
| Components | | | | | | |
| (weight %) | | | | | | |
| EGDCA | 54.3 | 54.3 | 54.3 | 54.3 | 54.3 | 56.35 |
| PEPEA | 11 | 11 | 11 | 11 | 11 | 12.5 |
| BCEA | 0 | 0 | 0 | 2.5 | 5 | 0 |
| HEMA | 15 | 14 | 15 | 12.5 | 10 | 15 |
| NBA | 14 | 15 | 15 | 14 | 14 | 12 |
| HEVB | 2.25 | 2.25 | 2.25 | 2.25 | 2.25 | 0 |
| UVB | 0 | 0 | 0 | 0 | 0 | 0.7 |
| TCDA | 3 | 3 | 3 | 3 | 3 | 3 |
| Omnirad 819 | 0.45 | 0.45 | 0.45 | 0.45 | 0.45 | 0.45 |
| Σ Components | 100 | 100 | 100 | 100 | 100 | 100 |
| Properties | | | | | | |
| RI (25) | 1.521339 | 1.520888 | 1.520862 | 1.520845 | 1.520221 | 1.520830 |
| | (0.000146) | (0.000133) | (0.000173) | (0.000106) | (0.000102) | (0.000055) |
| Abbe # (25) | 49.42 | 49.36 | 49.63 | 49.30 | 49.43 | 51.57 |
| | (0.45) | (0.62) | (0.61) | (0.46) | (0.39) | (0.63) |
| WC (wt. %) | 2.00 | 1.93 | 2.04 | 1.70 | 1.81 | 2.47 |
| | (0.08) | (0.04) | (0.02) | (0.02) | (0.18) | (0.43) |

Examples 33-38 exhibited both high refractive indices (greater than 1.50) and high Abbe numbers (greater than 50) making these materials most suitable for ophthalmic devices such as intraocular lenses. Examples 33-38 also exhibited low water contents which can provide dimensional stability for implantable ophthalmic devices.

### Examples 39-67

Polymer disks were fabricated using the standard curing, demolding, and extraction procedures (SCDEP) using the RMM listed in Tables 7-10. For each example, the refractive index, Abbe number, water content, tan deltas, and storage moduli were determined on the dried disks. The average refractive indexes, Abbe numbers, water contents, tan deltas, and storage moduli are also listed in Tables 7-10. Standard deviations are listed in parentheses.

TABLE 7
Formulations and Physical Properties

| | Ex. 39 | Ex. 40 | Ex. 41 | Ex. 42 | Ex. 43 | Ex. 44 | Ex. 45 |
|---|---|---|---|---|---|---|---|
| Components | | | | | | | |
| (weight %) | | | | | | | |
| EGDCA | 67.00 | 67.00 | 71.01 | 67.00 | 67.00 | 74.25 | 75.00 |
| OPEA | 9.25 | 7.25 | 4.19 | 10.00 | 0.00 | 0.00 | 1.25 |
| HBA | 18.00 | 18.00 | 14.01 | 17.25 | 18.00 | 18.00 | 18.00 |
| NHA | 4.00 | 4.00 | 8.01 | 4.00 | 11.25 | 4.00 | 4.00 |
| TCDA | 1.00 | 3.00 | 2.03 | 1.00 | 3.00 | 3.00 | 1.00 |
| HEVC | 0.30 | 0.30 | 0.30 | 0.30 | 0.30 | 0.30 | 0.30 |
| Omnirad 819 | 0.45 | 0.45 | 0.45 | 0.45 | 0.45 | 0.45 | 0.45 |
| Σ Components | 100.00 | 100.00 | 100.00 | 100.00 | 100.00 | 100.00 | 100.00 |
| Properties | | | | | | | |
| RI (25) | 1.518759 | 1.520018 | 1.519776 | 1.518773 | 1.517723 | 1.523164 | 1.522458 |
| | (0.000166) | (0.000124) | (0.000093) | (0.000170) | (0.000127) | (0.000144) | (0.000158) |
| ABBE # (25) | 51.97 | 51.63 | 52.30 | 52.00 | 52.01 | 52.52 | 52.13 |
| | (0.57) | (0.23) | (0.38) | (0.33) | (0.23) | (0.25) | (0.61) |
| WC (wt. %) | 1.37 | 1.47 | 1.06 | 1.34 | 1.14 | 0.99 | 1.59 |
| | (0.02) | (0.08) | (0.08) | (0.14) | (0.08) | (0.05) | (0.04) |
| Tan δₘₐₓ (° C.) | 25.2 | 24.4 | 24.4 | 24.5 | 18.7 | 27.3 | 26.2 |
| E′ (MPa) | 26.0 | 62.2 | 25.8 | 30.1 | 10.0 | 73.1 | 54.4 |

TABLE 8
Formulations and Physical Properties

| | Ex. 46 | Ex. 47 | Ex. 48 | Ex. 49 | Ex. 50 | Ex. 51 | Ex. 52 |
|---|---|---|---|---|---|---|---|
| Components | | | | | | | |
| (weight %) | | | | | | | |
| EGDCA | 74.25 | 67.00 | 67.00 | 67.00 | 75.00 | 75.00 | 75.00 |
| OPEA | 10.00 | 7.25 | 10.00 | 9.25 | 7.25 | 0.00 | 0.00 |
| HBA | 10.00 | 10.00 | 10.00 | 10.00 | 10.00 | 10.00 | 18.00 |
| NHA | 4.00 | 12.00 | 9.25 | 12.00 | 4.00 | 12.00 | 5.25 |
| TCDA | 1.00 | 3.00 | 3.00 | 1.00 | 3.00 | 2.25 | 1.00 |
| HEVC | 0.30 | 0.30 | 0.30 | 0.30 | 0.30 | 0.30 | 0.30 |
| Omnirad 819 | 0.45 | 0.45 | 0.45 | 0.45 | 0.45 | 0.45 | 0.45 |
| Σ Components | 100.00 | 100.00 | 100.00 | 100.00 | 100.00 | 100.00 | 100.00 |
| Properties | | | | | | | |
| RI (25) | 1.522324 | 1.517473 | 1.518518 | 1.516454 | 1.523967 | 1.520474 | 1.522202 |
| | (0.000101) | (0.000230) | (0.000140) | (0.000067) | (0.000136) | (0.000071) | (0.000238) |
| ABBE # (25) | 52.15 | 52.03 | 51.82 | 52.18 | 52.34 | 52.12 | 52.53 |
| | (0.83) | (0.64) | (0.25) | (0.35) | (0.39) | (0.33) | (0.16) |
| WC (wt. %) | 1.27 | 1.03 | 1.23 | 1.19 | 1.22 | 0.93 | 1.64 |
| | (0.02) | (0.03) | (0.08) | (0.12) | (0.05) | (0.12) | (0.05) |
| Tan δₘₐₓ (° C.) | 29.4 | 22.8 | 25.1 | 19.7 | 31.5 | 22.2 | 25.3 |
| E′ (MPa) | 164 | 17.5 | 39.2 | 10.5 | 271 | 16.9 | 35.2 |

TABLE 9
Formulations and Physical Properties

| | Ex. 53 | Ex. 54 | Ex. 55 | Ex. 56 | Ex. 57 | Ex. 58 | Ex. 59 |
|---|---|---|---|---|---|---|---|
| Components | | | | | | | |
| (weight %) | | | | | | | |
| EGDCA | 67.00 | 75.00 | 74.25 | 72.25 | 75.00 | 75.00 | 67.00 |
| OPEA | 0.00 | 9.25 | 0.00 | 10.00 | 0.00 | 0.00 | 1.25 |
| HBA | 17.25 | 10.00 | 10.00 | 10.00 | 10.00 | 11.25 | 18.00 |
| NHA | 12.00 | 4.00 | 12.00 | 4.00 | 11.25 | 12.00 | 12.00 |
| TCDA | 3.00 | 1.00 | 3.00 | 3.00 | 3.00 | 1.00 | 1.00 |
| HEVC | 0.30 | 0.30 | 0.30 | 0.30 | 0.30 | 0.30 | 0.30 |
| Omnirad 819 | 0.45 | 0.45 | 0.45 | 0.45 | 0.45 | 0.45 | 0.45 |
| Σ Components | 100.00 | 100.00 | 100.00 | 100.00 | 100.00 | 100.00 | 100.00 |
| Properties | | | | | | | |
| RI (25) | 1.517820 | 1.522384 | 1.520670 | 1.522834 | 1.521069 | 1.519727 | 1.516091 |
| | (0.000138) | (0.000181) | (0.000112) | (0.000151) | (0.000147) | (0.000096) | (0.000076) |
| ABBE # (25) | 52.45 | 52.24 | 52.16 | 52.23 | 51.66 | 52.25 | 52.76 |
| | (0.52) | (0.46) | (0.08) | (0.28) | (0.16) | (0.16) | (0.26) |
| WC (wt. %) | 1.54 | 1.35 | 1.00 | 1.36 | 0.97 | 1.12 | 1.61 |
| | (0.10) | (0.14) | (0.14) | (0.01) | (0.06) | (0.02) | (0.05) |
| Tan δₘₐₓ (° C.) | 19.3 | 28.9 | 22.6 | 30.7 | 23.5 | 19.9 | 16.7 |
| E′ (MPa) | 11.0 | 145 | 23.2 | 200 | 26.2 | 9.1 | 6.1 |

TABLE 10
Formulations and Physical Properties

| | Ex. 60 | Ex. 61 | Ex. 62 | Ex. 63 | Ex. 64 | Ex. 65 | Ex. 66 | Ex. 67 |
|---|---|---|---|---|---|---|---|---|
| Components | | | | | | | | |
| (weight %) | | | | | | | | |
| EGDCA | 68.25 | 75.00 | 67.00 | 75.00 | 75.00 | 67.00 | 67.00 | 71.01 |
| OPEA | 0.00 | 1.25 | 10.00 | 0.00 | 0.00 | 10.00 | 0.00 | 4.19 |
| HBA | 18.00 | 10.00 | 15.25 | 17.25 | 18.00 | 10.00 | 18.00 | 14.01 |
| NHA | 12.00 | 12.00 | 4.00 | 4.00 | 4.00 | 11.25 | 12.00 | 8.01 |
| TCDA | 1.00 | 1.00 | 3.00 | 3.00 | 2.25 | 1.00 | 2.25 | 2.03 |
| HEVC | 0.30 | 0.30 | 0.30 | 0.30 | 0.30 | 0.30 | 0.30 | 0.30 |
| Omnirad 819 | 0.45 | 0.45 | 0.45 | 0.45 | 0.45 | 0.45 | 0.45 | 0.45 |
| Σ Components | 100.00 | 100.00 | 100.00 | 100.00 | 100.00 | 100.00 | 100.00 | 100.00 |
| Properties | | | | | | | | |
| RI (25) | 1.517038 | 1.519577 | 1.520462 | 1.523580 | 1.523456 | 1.516684 | 1.517274 | 1.520042 |
| | (0.000098) | (0.000051) | (0.000256) | (0.000154) | (0.000111) | (0.000089) | (0.000147) | (0.000161) |
| ABBE # (25) | 52.64 | 52.46 | 52.14 | 51.97 | 52.56 | 52.38 | 52.19 | 51.69 |
| | (0.33) | (0.25) | (0.33) | (0.39) | (0.21) | (0.30) | (0.25) | (0.40) |
| WC (wt. %) | 1.59 | 1.03 | 1.96 | 1.31 | 1.32 | 1.09 | 1.45 | 1.39 |
| | (0.01) | (0.07) | (0.11) | (0.06) | (0.04) | (0.08) | (0.06) | (0.07) |
| Tan δₘₐₓ (° C.) | 17.6 | 20.2 | 27.8 | 29.1 | 27.5 | 20.7 | 18 | 23.9 |
| E′ (MPa) | 6.9 | 11.0 | 107 | 128 | 84.5 | 10.7 | 7.7 | 24.7 |

Examples 39-67 exhibited both high refractive indices (greater than 1.51) and high Abbe number (greater than 51.50) making these materials most suitable for ophthalmic devices such as intraocular lenses. Examples 39-67 also exhibited low water contents (between 1-2 weight percent) which can provide dimensional stability for implantable ophthalmic devices.

### Examples 68-73

Polymer disks were fabricated using the standard curing, demolding, and extraction procedures (SCDEP) using the RMM listed in Table 11. For each example, the refractive index, Abbe number, water content, tan deltas, and storage moduli were determined on the dried disks. The average refractive indexes, Abbe numbers, water contents, tan deltas, and storage moduli are also listed in Tables 11. Standard deviations are listed in parentheses.

TABLE 11
Formulations and Physical Properties

| | Ex. 68 | Ex. 69 | Ex. 70 | Ex. 71 | Ex. 72 | Ex. 73 |
|---|---|---|---|---|---|---|
| Components | | | | | | |
| (weight %) | | | | | | |
| EGDCA | 70 | 70 | 70 | 70 | 70 | 70 |
| BCEA | 9 | 9 | 9 | 9 | 9 | 9 |
| NBA | 6.3 | 6.3 | 6.3 | 6.3 | 6.3 | 6.3 |
| EGDMA | 1 | 1 | 1 | 1 | 1 | 1 |
| TCDA | 3 | 3 | 3 | 3 | 3 | 3 |
| HEVB | 2.25 | 2.25 | 2.25 | 2.25 | 2.25 | 2.25 |
| Omnirad 819 | 0.45 | 0.45 | 0.45 | 0.45 | 0.45 | 0.45 |
| PEG-OH—N4 | 8 | 0 | 0 | 0 | 0 | 0 |
| PEG-OH—N5 | 0 | 8 | 0 | 0 | 0 | 0 |
| PEG-OH—N6 | 0 | 0 | 8 | 0 | 0 | 0 |
| PEG-OH—N7 | 0 | 0 | 0 | 8 | 0 | 0 |
| PEG-OH—N8 | 0 | 0 | 0 | 0 | 8 | 0 |
| PEG-OH-MIX1 | 0 | 0 | 0 | 0 | 0 | 8 |
| Σ Components | 100 | 100 | 100 | 100 | 100 | 100 |
| Properties | | | | | | |
| RI (25) | 1.523876 | 1.523419 | 1.523696 | 1.523345 | 1.523055 | 1.523298 |
| | (0.000071) | (0.000314) | (0.000074) | (0.000056) | (0.000159) | (0.000107) |
| Abbe # (25) | 47.76 | 49.15 | 48.44 | 47.99 | 48.80 | 49.23 |
| | (0.37) | (0.95) | (0.24) | (0.34) | (0.62) | (0.43) |
| WC (wt. %) | 1.23 | 1.14 | 1.24 | 1.32 | 1.29 | 1.31 |
| | (0.05) | (0.01) | (0.01) | (0.13) | (0.04) | (0.03) |
| Tan δₘₐₓ (° C.) | 30.7 | 29.6 | 28.2 | 27.3 | 27.1 | 27.9 |
| E′ (MPa) | 211 | 147 | 112 | 73.2 | 71.7 | 99.6 |
| | (0.3) | (0.2) | (0.2) | (0.1) | (0.1) | (0.3) |

Examples 68-73 exhibited high refractive indices (greater than 1.51) and Abbe numbers between 47.5 and 49.5 making these materials suitable for ophthalmic devices such as intraocular lenses. Examples 68-73 also exhibited low water contents (around 1 weight percent) which can provide dimensional stability for implantable ophthalmic devices.

### Examples 74-82

Polymer disks were fabricated using the standard curing, demolding, and extraction procedures (SCDEP) using the RMM listed in Tables 12 and 13. For each example, the refractive index, Abbe number, water content, tan deltas, and storage moduli were determined on the dried disks. The average refractive indexes, Abbe numbers, water contents, tan deltas, and storage moduli are also listed in Tables 12 and 13. Standard deviations are listed in parentheses.

TABLE 12
Formulations and Physical Properties

| Components | | | | | |
|---|---|---|---|---|---|
| (weight %) | Ex. 74 | Ex. 75 | Ex. 76 | Ex. 77 | Ex. 78 |
| EGDCA | 74.7307 | 74.7307 | 71.7307 | 69.7307 | 74.7307 |
| BCEA | 6 | 6 | 6 | 6 | 6 |
| PEG-OH-MIX | 0 | 11.7307 | 0 | 0 | 0 |
| HBA | 11.7307 | 0 | 14.7307 | 16.7307 | 11.7307 |
| NHA | 4.8 | 4.8 | 4.8 | 4.8 | 5.2886 |
| TCDA | 1.9886 | 1.9886 | 1.9886 | 1.9886 | 1.5 |
| HEVB | 0 | 0 | 0 | 0 | 0 |
| HEVC | 0.3 | 0.3 | 0.3 | 0.3 | 0.3 |
| Omnirad 819 | 0.45 | 0.45 | 0.45 | 0.45 | 0.45 |
| Σ Components | 100 | 100 | 100 | 100 | 100 |

| Properties | Ex. 74 | Ex. 75 | Ex. 76 | Ex. 77 | Ex. 78 |
|---|---|---|---|---|---|
| RI (25) | 1.522312 | 1.519591 | 1.521101 | 1.520575 | 1.521727 |
| | (0.000066) | (0.000171) | (0.000167) | (0.000220) | (0.000097) |
| Abbe # (25) | 52.84 | 54.52 | 52.80 | 52.27 | 52.16 |
| | (0.24) | (1.18) | (0.41) | (0.48) | (0.28) |
| WC (wt. %) | 1.34 | 2.23 | 1.60 | 1.57 | 1.27 |
| | (0.04) | (0.03) | (0.05) | (0.18) | (0.05) |
| Tan δₘₐₓ (° C.) | 26.8 | 20.5 | 25.5 | 24.6 | 26.1 |
| E′ (MPa) | 52.9 | 13.9 | 55.6 | 36.9 | 57.9 |
| | (1) | (0.04) | (0.9) | (0.9) | (0.9) |

TABLE 13
Formulations and Physical Properties

| Components | | | | |
|---|---|---|---|---|
| (weight %) | Ex. 79 | Ex. 80 | Ex. 81 | Ex. 82 |
| EGDCA | 74.7307 | 74.7307 | 72.7807 | 72.7807 |
| BCEA | 6 | 6 | 6 | 6 |
| PEG-OH-MIX | 0 | 0 | 0 | 0 |
| HBA | 11.7307 | 11.7307 | 11.7307 | 11.7307 |
| NHA | 5.7886 | 6.2886 | 5.7886 | 6.2886 |
| TCDA | 1 | 0.5 | 1 | 0.5 |
| HEVB | 0 | 0 | 2.25 | 2.25 |
| HEVC | 0.3 | 0.3 | 0 | 0 |
| Omnirad 819 | 0.45 | 0.45 | 0.45 | 0.45 |
| Σ Components | 100 | 100 | 100 | 100 |

| Properties | Ex. 78 | Ex. 79 | Ex. 81 | Ex. 82 |
|---|---|---|---|---|
| RI (25) | 1.521536 | 1.520856 | 1.524635 | 1.524117 |
| | (0.000168) | (0.000343) | (0.000068) | (0.000058) |
| Abbe # (25) | 52.44 | 52.51 | 48.16 | 48.33 |
| | (0.21) | (0.33) | (0.30) | (0.18) |
| WC (wt. %) | 1.26 | 1.30 | 0.90 | 0.92 |
| | (0.04) | (0.03) | (0.05) | (0.03) |
| Tan δₘₐₓ (° C.) | 24.6 | 23.5 | 28.9 | 26.9 |
| E′ (MPa) | 34.4 | 23.4 | 93.2 | 73.9 |
| | (0.5) | (0.4) | (2) | (2) |

Examples 74-80 containing between 69-75 weight percent EGDCA and 0.3 weight percent HEVC exhibited high refractive indices (greater than 1.51) and Abbe numbers (greater than 52) making these materials most suitable for ophthalmic devices such as intraocular lenses. Examples 81 and 82 containing about 73 weight percent EGDCA and 2.25 weight percent HEVB exhibited high refractive indices (greater than 1.52), but the Abbe numbers decreased to about 48, making these materials suitable for ophthalmic devices such as intraocular lenses.

Examples 74-82 also exhibited low water contents (between about 0.9-2.5 weight percent) which can provide dimensional stability for implantable ophthalmic devices.

### Examples 83-87

Polymer disks were fabricated using the standard curing, demolding, and extraction procedures (SCDEP) using the RMM listed in Table 14. For each example, the refractive index, Abbe number, water content, tan deltas, and storage moduli were determined on the dried disks. The average refractive indexes, Abbe numbers, water contents, tan deltas, and storage moduli are also listed in Tables 14. Standard deviations are listed in parentheses.

TABLE 14
Formulations and Physical Properties

| Components | | | | | |
|---|---|---|---|---|---|
| (weight %) | Ex. 83 | Ex. 84 | Ex. 85 | Ex. 86 | Ex. 87 |
| EGDCA | 74.7307 | 74.7307 | 75.25 | 75.25 | 76.25 |
| BCEA | 6 | 4 | 5 | 5 | 4.5 |
| HBA | 10 | 11.7307 | 11.75 | 11.75 | 11.75 |
| NHA | 6.5307 | 6.8 | 4.75 | 4.75 | 4.75 |
| TCDA | 1.9886 | 1.9886 | 2 | 2 | 2 |
| HEVC | 0.3 | 0.3 | 0.3 | 0.3 | 0.3 |
| Omnirad 819 | 0.45 | 0.45 | 0.45 | 0.45 | 0.45 |
| Σ Components | 100 | 100 | 100 | 100 | 100 |

| Properties | Ex. 83 | Ex. 84 | Ex. 85 | Ex. 86 | Ex. 87 |
|---|---|---|---|---|---|
| RI (25) | 1.521683 | 1.521791 | 1.522717 | 1.523074 | 1.523437 |
| | (0.000054) | (0.000126) | (0.000120) | (0.000160) | (0.000161) |
| Abbe # (25) | 51.62 | 51.64 | 51.34 | 51.46 | 51.85 |
| | (0.24) | (0.16) | (0.19) | (0.37) | (0.36) |
| WC (wt. %) | 1.33 | 1.09 | 1.47 | 1.07 | 1.14 |
| | (0.12) | (0.10) | (0.25) | (0.06) | (0.12) |
| Tan δₘₐₓ (° C.) | 26.6 | 26.1 | 28 | 27.1 | 28.7 |
| E′ (MPa) | 61 | 57.4 | 132 | 133 | 141 |

Examples 83-87 exhibited high refractive indices (greater than 1.52) and Abbe numbers (greater than 51) making these materials most suitable for ophthalmic devices such as intraocular lenses. Examples 83-87 also exhibited low water contents (about 1-1.5 weight percent) which can provide dimensional stability for implantable ophthalmic devices.

### Examples 87-91

Polymer disks were fabricated using the standard curing, demolding, and extraction procedures (SCDEP) using the RMM listed in Table 15. For each example, the refractive index, Abbe number, water content, tan deltas, and storage moduli were determined on the dried disks. The average refractive indexes, Abbe numbers, water contents, tan deltas, and storage moduli are also listed in Table 15. Standard deviations are listed in parentheses.

TABLE 15
Formulations and Physical Properties

| Components | | | | | |
|---|---|---|---|---|---|
| (weight %) | Ex. 88 | Ex. 89 | Ex. 90 | Ex. 91 | Ex. 92 |
| EGDCA | 77.25 | 77.25 | 77.25 | 78 | 78 |
| HBA | 15 | 14.5 | 14 | 13.25 | 12.75 |
| NHA | 5 | 5.5 | 6 | 6 | 6.5 |
| TCDA | 2 | 2 | 2 | 2 | 2 |
| HEVC | 0.3 | 0.3 | 0.3 | 0.3 | 0.3 |
| Omnirad 819 | 0.45 | 0.45 | 0.45 | 0.45 | 0.45 |
| Σ Components | 100 | 100 | 100 | 100 | 100 |

| Properties | Ex. 88 | Ex. 89 | Ex. 90 | Ex. 91 | Ex. 92 |
|---|---|---|---|---|---|
| RI (25) | 1.523546 | 1.523707 | 1.523610 | 1.524074 | 1.523909 |
| | (0.000086) | (0.000116) | (0.000078) | (0.000063) | (0.000097) |
| Abbe # (25) | 52.13 | 52.49 | 52.35 | 52.63 | 52.23 |
| | (0.27) | (0.30) | (0.77) | (0.31) | (0.35) |
| WC (wt. %) | 1.10 | 1.23 | 1.23 | 1.33 | 1.05 |
| | (0.15) | (0.02) | (0.17) | (0.01) | (0.07) |
| Tan δₘₐₓ (° C.) | 28 | 27.5 | 26.6 | 28 | 27.8 |
| E′ (MPa) | 53.8 | 80.3 | 72.4 | 92.5 | 85.5 |
| | (0.2) | (0.2) | (0.2) | (0.2) | (0.2) |

Examples 88-92 exhibited high refractive indices (greater than 1.52) and Abbe numbers (greater than 52) making these materials most suitable for ophthalmic devices such as intraocular lenses. Examples 88-92 also exhibited low water contents (about 1-1.5 weight percent) which can provide dimensional stability for implantable ophthalmic devices.

### Examples 93-102

Polymer disks were fabricated using the standard curing, demolding, and extraction procedures (SCDEP) using the RMM listed in Tables 16 and 17. For each example, the refractive index, Abbe number, water content, tan deltas, and storage moduli were determined on the dried disks. The average refractive indexes, Abbe numbers, water contents, tan deltas, and storage moduli are also listed in Tables 16 and 17. Standard deviations are listed in parentheses.

TABLE 16
Formulations and Physical Properties

| Components | | | | | |
|---|---|---|---|---|---|
| (weight %) | Ex. 93 | Ex. 94 | Ex. 95 | Ex. 96 | Ex. 97 |
| EGDCA | 72.05 | 72.05 | 72.5 | 72.5 | 72.5 |
| BCEA | 9 | 9 | 0 | 0 | 0 |
| OPEA | 0 | 0 | 9.5 | 9.5 | 9.5 |
| NHA | 7.3 | 7.3 | 6.35 | 6.35 | 6.35 |
| TCDA | 3 | 3 | 1 | 1 | 1 |
| HEVC | 0.2 | 0.2 | 0.2 | 0.2 | 0.2 |
| PEG-OH—N6 | 0 | 8 | 0 | 0 | 10 |
| PEG-OH—N7 | 0 | 0 | 0 | 10 | 0 |
| PEG-OH—N8 | 0 | 0 | 10 | 0 | 0 |
| PEG-OH-MIX2 | 8 | 0 | 0 | 0 | 0 |
| Omnirad 819 | 0.45 | 0.45 | 0.45 | 0.45 | 0.45 |
| Σ Components | 100 | 100 | 100 | 100 | 100 |

| Properties | Ex. 93 | Ex. 94 | Ex. 95 | Ex. 96 | Ex. 97 |
|---|---|---|---|---|---|
| RI (25) | 1.518356 | 1.518779 | 1.517213 | 1.518483 | 1.517708 |
| | (0.000177) | (0.000119) | (0.000308) | (0.001366) | (0.000229) |
| Abbe # (25) | 52.92 | 53.07 | 52.90 | 52.77 | 53.34 |
| | (0.35) | (0.67) | (0.18) | (0.51) | (0.56) |
| WC (wt. %) | 0.98 | 1.14 | 2.39 | 2.08 | 1.41 |
| | (0.02) | (0.09) | (0.11) | (0.06) | (0.05) |
| Tan δₘₐₓ (° C.) | 21.9 | 21.3 | 19.8 | 20.4 | 21 |
| E′ (MPa) | 15.8 | 16.1 | 10.1 | 11.1 | 14.3 |
| | (0.2) | (0.3) | (0.2) | (0.2) | (0.3) |

TABLE 17
Formulations and Physical Properties

| Components | | | | | |
|---|---|---|---|---|---|
| (weight %) | Ex. 98 | Ex. 99 | Ex. 100 | Ex. 101 | Ex. 102 |
| EGDCA | 72.5 | 72.5 | 72.5 | 72.5 | 72.5 |
| BCEA | 0 | 0 | 0 | 0 | 0 |
| OPEA | 9.5 | 9.5 | 9.5 | 9.5 | 9.5 |
| NHA | 6.35 | 6.35 | 6.35 | 6.35 | 6.35 |
| TCDA | 1 | 1 | 1 | 1 | 1 |
| HEVC | 0.2 | 0.2 | 0.2 | 0.2 | 0.2 |
| PEG-OH—N2 | 0 | 0 | 0 | 10 | 0 |
| PEG-OH—N3 | 0 | 0 | 10 | 0 | 0 |
| PEG-OH—N4 | 0 | 10 | 0 | 0 | 0 |
| PEG-OH—N5 | 10 | 0 | 0 | 0 | 0 |
| PEG-OH-MIX | 0 | 0 | 0 | 0 | 10 |
| Omnirad 819 | 0.45 | 0.45 | 0.45 | 0.45 | 0.45 |
| Σ Components | 100 | 100 | 100 | 100 | 100 |

| Properties | Ex. 98 | Ex. 99 | Ex. 100 | Ex. 101 | Ex. 102 |
|---|---|---|---|---|---|
| RI (25) | 1.518321 | 1.518556 | 1.520106 | 1.520048 | 1.517120 |
| | (0.000080) | (0.000190) | (0.000089) | (0.000156) | (0.000653) |
| Abbe # (25) | 53.22 | 53.28 | 53.09 | 52.80 | 54.89 |
| | (0.13) | (0.46) | (0.64) | (0.97) | (2.57) |
| WC (wt. %) | 1.47 | 1.32 | 1.19 | 1.07 | 1.66 |
| | (0.09) | (0.16) | (0.05) | (0.10) | (0.09) |
| Tan δₘₐₓ (° C.) | 23.3 | 23.1 | 25 | 30 | 21.4 |
| E′ (MPa) | 22.0 | 20.4 | 40.3 | 175.0 | 13.6 |
| | (0.7) | (0.5) | (0.8) | (0.5) | (0.2) |

Examples 93-102 exhibited high refractive indices (greater than 1.51) and Abbe numbers (greater than 52) making these materials most suitable for ophthalmic devices such as intraocular lenses. Examples 93-102 also exhibited low water contents (about 1-2.5 weight percent) which can provide dimensional stability for implantable ophthalmic devices.

Inhibitor Removal from Ethylene Glycol Dicyclopentenyl Ether Acrylate (EGDCA)

In examples 104-151, the inhibitor monomethyl ether hydroquinone (MEHQ) was removed from the monomer EGDCA before use by preparative column chromatography using activated basic alumina (Brockman Grade 1, 58 nanometers, Millipore Sigma #199443-5). The activated basic alumina was first dried at 200° C. or at 70° C. under 50 millibars for 22 hours, and then, in a fume hood, transferred into a 25×600 millimeters Kontes Chromaflex column (30 grams of dried alumina per 100 mL of EGDCA) equipped with a threaded cap and air pressure connection. The alumina was covered with 1-2 centimeters of sand and then the EGDCA was added to the column, thereafter the column was capped. Air pressure was applied to create a filtration rate of 2-3 drops per second (about 7 psi). The de-inhibited EGDCA was collected in an amber bottle and either used immediately or stored in the refrigerator.

### Examples 103-108

Polymer disks were fabricated using the standard curing and demolding procedures (SCDEP) using the RMM listed in Table 18 and then extracted with acetonitrile (AAEP). Extracted disks were vacuum dried at 85° C. (<1 in Hg) to constant weight and then stored under a nitrogen gas atmosphere in a glove box at room temperature until the time of analysis. For each example, the refractive index, Abbe number, tan deltas, and storage moduli were determined on the extracted, dried disks (denoted as “dry” in Table 18). The average refractive indexes, Abbe numbers, tan deltas, and storage moduli are listed in Table 18. Standard deviations are listed in parentheses. Some disks were subsequently hydrated for 24 hours in deionized water at ambient temperature before the physical and mechanical properties were measured to determine the impact of water content and storage conditions (denoted as “hydrated” in Table 18).

TABLE 18
Formulations and Physical Properties

| | Ex. 103 | Ex. 104 | Ex. 105 | Ex. 106 | Ex. 107 | Ex. 108 |
|---|---|---|---|---|---|---|
| Components | | | | | | |
| (weight %) | | | | | | |
| EGDCA | 72.5 | 72.5 | 72 | 71.67 | 71.42 | 75 |
| OPEA | 9.5 | 9.5 | 9 | 8.68 | 8.43 | 1.25 |
| HBA | 10 | 10 | 10 | 10 | 10 | 18 |
| NHA | 6.35 | 6.35 | 7.35 | 8 | 8.5 | 4 |
| TCDA | 1 | 1 | 1 | 1 | 1 | 1 |
| HEVC | 0.2 | 0.2 | 0.2 | 0.2 | 0.2 | 0.3 |
| Omnirad 819 | 0.45 | 0.45 | 0.45 | 0.45 | 0.45 | 0.45 |
| Σ Components | 100 | 100 | 100 | 100 | 100 | 100 |
| Properties | | | | | | |
| RI (25) | 1.521402 | 1.521257 | 1.520635 | 1.520189 | 1.519906 | 1.522885 |
| | (0.000121) | (0.000101) | (0.000030) | (0.000129) | (0.000241) | (0.000144) |
| Abbe # (25) | 52.07 | 52.52 | 52.54 | 52.05 | 52.26 | 52.07 |
| | (0.24) | (0.23) | (0.18) | (0.46) | (0.46) | (0.36) |
| Tan δₘₐₓ (° C.) | 30.4 | 30.1 | 28.6 | 28.1 | 27.2 | 30.6 |
| Dry | | | | | | |
| Tan δₘₐₓ (° C.) | 25.5 | 24.6 | 24.0 | 22.3 | 22.1 | 24.7 |
| Hydrated | | | | | | |
| E′ (MPa) | 258 | 217 | 155 | 88.8 | 73.2 | 334 |
| Dry | (0.6) | (0.6) | (0.3) | (0.3) | (0.3) | (0.6) |
| E′ (MPa) | 29.0 | 21.8 | 16.3 | 11.0 | 12.0 | 17.3 |
| Hydrated | (0.5) | (0.5) | (0.4) | (0.8) | (0.3) | (0.4) |

Examples 103-108 exhibited high refractive indices (greater than 1.51) and Abbe numbers (greater than 52) making these materials most suitable for ophthalmic devices such as intraocular lenses. The hydrated materials of examples 103-108 exhibited lower tan δmax and E′ as compared to the corresponding dry materials, suggesting that these physical properties are dependent on the water content and therefore on the post-lens fabrication conditions such as drying conditions and relative humidity. Equilibrating in deionized water before sterilization may minimize or eliminate any dependency on water content.

### Examples 109-113

Polymer disks were fabricated using the standard curing and demolding procedures (SCDEP) using the RMM listed in Table 19 and then extracted with acetonitrile (AAEP). Extracted disks were vacuum dried at 850 (1 in Hg) to constant weight and then stored under a nitrogen gas atmosphere in a glove box at room temperature until the time of analysis. For each example, the refractive index, Abbe number, tan deltas, and storage moduli were determined on the extracted, dried disks (denoted as “dry” in Table 19 and equivalent to zero percent humidity). The average refractive indexes, Abbe numbers, tan deltas, and storage moduli are listed in Table 19. Standard deviations are listed in parentheses. Some disks were subsequently stored under controlled relative humidity (25% or 4500 relative humidity) at ambient temperature for 24 hours before the physical and mechanical properties were measured to determine the impact of relative humidity on physical properties (denoted as “RH25 or RH45” in Table 19).

TABLE 19
Formulations and Physical Properties

| Components | | | | | |
|---|---|---|---|---|---|
| (weight %) | Ex. 109 | Ex. 110 | Ex. 111 | Ex. 112 | Ex. 113 |
| EGDCA | 74.7803 | 74.7803 | 74.7807 | 74.7807 | 70 |
| BCEA | 6 | 8 | 10 | 12.2886 | 14.85 |
| HBA | 11.7803 | 11.7803 | 11.7807 | 11.7807 | 14 |
| NHA | 6.2886 | 4.2886 | 2.2886 | 0 | 0 |
| TCDA | 0.5 | 0.5 | 0.5 | 0.5 | 0.5 |
| HEVC | 0.2 | 0.2 | 0.2 | 0.2 | 0.2 |
| Omnirad 819 | 0.45 | 0.45 | 0.45 | 0.45 | 0.45 |
| Σ Components | 100 | 100 | 100 | 100 | 100 |

| Properties | Ex. 109 | Ex. 110 | Ex. 111 | Ex. 112 | Ex. 113 |
|---|---|---|---|---|---|
| RI (25) | 1.521592 | 1.522140 | 1.522873 | 1.523588 | 1.521422 |
| | (0.000099) | (0.000149) | (0.000127) | (0.000318) | (0.000328) |
| Abbe # (25) | 52.60 | 52.75 | 52.58 | 52.70 | 52.53 |
| | (0.15) | (0.19) | (0.32) | (0.41) | (0.87) |
| Tan δₘₐₓ (° C.) | 27.8 | 30.1 | 31.1 | 33.7 | 32 |
| Dry | | | | | |
| Tan δₘₐₓ (° C.) | 27.1 | 28.5 | 30 | — | — |
| RH25 | | | | | |
| Tan δₘₐₓ (° C.) | 26.1 | 27.2 | 29 | — | — |
| RH45 | | | | | |
| E′ (MPa) | 138 | 242 | 360 | 563 | 496 |
| Dry | (0.3) | (0.8) | (0.2) | (0.7) | (0.5) |
| E′ (MPa) | 71.6 | 136 | 257 | — | — |
| RH25 | (0.1) | (0.1) | (0.5) | | |
| E′ (MPa) | 30.8 | 79.1 | 141 | — | — |
| RH45 | (0.5) | (0.1) | (0.2) | | |

Examples 109-113 exhibited high refractive indices (greater than 1.52) and Abbe numbers (greater than 52) making these materials most suitable for ophthalmic devices such as intraocular lenses. For examples 109-113, samples equilibrated in relative humidity of 25% or 45% exhibited lower tan δmax and E′ as compared to the corresponding dry materials, suggesting that these physical properties are dependent on the water content and therefore on the post-lens fabrication conditions such as drying conditions and relative humidity. Equilibrating in deionized water before sterilization may minimize or eliminate any dependency on water content.

### Examples 114-119

Polymer disks were fabricated using the standard curing and demolding procedures (SCDEP) using the RMM listed in Table 20 and then extracted with acetonitrile (AAEP). Extracted disks were vacuum dried at 85° C. (<1 in Hg) to constant weight and then stored under a nitrogen gas atmosphere in a glove box at room temperature until the time of analysis. For each example, the refractive index, Abbe number, tan deltas, and storage moduli were determined on the extracted, dried disks (denoted as “dry” in Table 20 and equivalent to zero percent humidity). The average refractive indexes, Abbe numbers, water contents, tan deltas, and storage moduli are listed in Table 20. Standard deviations are listed in parentheses. Some disks were subsequently stored under controlled relative humidity (25% or 45% relative humidity) at ambient temperature for 24 hours before the physical and mechanical properties were measured to determine the impact of relative humidity on physical properties (denoted as “RH25 or RH45” in Table 20). Some disks were subsequently hydrated for 24 hours in deionized water at 37° C. before the physical and mechanical properties were measured to determine the impact of water content and storage conditions (denoted as “hydrated” in Table 20).

TABLE 20
Formulations and Physical Properties

| | Ex. 114 | Ex. 115 | Ex. 116 | Ex. 117 | Ex. 118 | Ex. 119 |
|---|---|---|---|---|---|---|
| Components | | | | | | |
| (weight %) | | | | | | |
| EGDCA | 68 | 70 | 68 | 65 | 60 | 60 |
| BCEA | 18.85 | 12.35 | 14.35 | 17.35 | 22.35 | 22.6 |
| HBA | 12 | 12 | 12 | 12 | 12 | 12 |
| NHA | 0 | 4.5 | 4.5 | 4.5 | 4.5 | 4.5 |
| TCDA | 0.5 | 0.5 | 0.5 | 0.5 | 0.5 | 0.25 |
| HEVC | 0.2 | 0.2 | 0.2 | 0.2 | 0.2 | 0.2 |
| Omnirad 819 | 0.45 | 0.45 | 0.45 | 0.45 | 0.45 | 0.45 |
| Σ Components | 100 | 100 | 100 | 100 | 100 | 100 |
| Properties | | | | | | |
| RI (25) | 1.520338 | 1.520024 | 1.519049 | 1.516981 | 1.514776 | 1.514970 |
| | (0.000064) | (0.000103) | (0.000071) | (0.000107) | (0.000258) | (0.000191) |
| Abbe # (25) | 53.19 | 51.90 | 52.14 | 53.16 | 52.50 | 53.43 |
| | (0.14) | (0.42) | (0.24) | (0.24) | (0.37) | (0.54) |
| WC (wt. %) | 1.69 | 1.73 | 1.61 | 1.34 | 1.55 | 1.76 |
| Hydrated | (0.05) | (0.20) | (0.03) | (0.07) | (0.19) | (0.02) |
| Tan δₘₐₓ (° C.) | 31.3 | 28.4 | 27.4 | 27 | 25.6 | 25.2 |
| Dry | | | | | | |
| Tan δₘₐₓ (° C.) | — | 26.4 | 25.6 | — | — | — |
| RH25 | | | | | | |
| Tan δₘₐₓ (° C.) | — | 25.3 | 24.6 | — | — | — |
| RH45 | | | | | | |
| E′ (MPa) | 454 | 126 | 112 | 54.8 | 57.0 | 51.4 |
| Dry | (0.5) | (0.4) | (0.2) | (0.6) | (1.0) | (1.7) |
| E′ (MPa) | — | 52.4 | 44.7 | — | — | — |
| RH25 | | (0.60 | (0.1) | | | |
| E′ (MPa) | — | 38.2 | 35.6 | — | — | — |
| RH45 | | (0.9) | (0.4) | | | |

Examples 114-119 exhibited high refractive indices (greater than 1.51) and Abbe numbers (greater than 51) making these materials most suitable for ophthalmic devices such as intraocular lenses. For examples 115 and 116, samples equilibrated in relative humidity of 25% or 45% exhibited lower tan δmax and E′ as compared to the corresponding dry materials, suggesting that these physical properties are dependent on the water content and therefore on the post-lens fabrication conditions such as drying conditions and relative humidity. Equilibrating in deionized water before sterilization may minimize or eliminate any dependency on water content.

### Examples 120-130

Polymer disks were fabricated using the standard curing and demolding procedures (SCDEP) using the RMM listed in Tables 21 and 22 and then extracted with acetonitrile (AAEP). Extracted disks were vacuum dried at 85 C (<1 in Hg) to constant weight and then stored under a nitrogen gas atmosphere in a glove box at room temperature until the time of analysis. For each example, the refractive index, Abbe number, tan deltas, and storage moduli were determined on the extracted, dried disks (denoted as “dry” in Tables 21 and 22). The average refractive indexes, Abbe numbers, tan deltas, and storage moduli are listed in Tables 21 and 22. Standard deviations are listed in parentheses. Some disks were subsequently hydrated for 2 weeks in deionized water at 37° C. before the physical and mechanical properties were measured to determine the impact of water content and storage conditions (denoted as “hydrated” in Tables 21 and 22).

TABLE 20
Formulations and Physical Properties

| | Ex. 120 | Ex. 121 | Ex. 122 | Ex. 123 | Ex. 124 | Ex. 125 |
|---|---|---|---|---|---|---|
| Components | | | | | | |
| (weight %) | | | | | | |
| EGDCA | 30 | 40 | 50 | 55 | 57.5 | 57.5 |
| BCEA | 52.35 | 42.35 | 32.35 | 27.35 | 24.85 | 25 |
| HBA | 11 | 11 | 11 | 11 | 11 | 10 |
| NHA | 5.5 | 5.5 | 5.5 | 5.5 | 5.5 | 5.85 |
| TCDA | 0.5 | 0.5 | 0.5 | 0.5 | 0.5 | 1 |
| HEVC | 0.2 | 0.2 | 0.2 | 0.2 | 0.2 | 0.2 |
| Omnirad 819 | 0.45 | 0.45 | 0.45 | 0.45 | 0.45 | 0.45 |
| Σ Components | 100 | 100 | 100 | 100 | 100 | 100 |
| Properties | | | | | | |
| RI (25) | 1.500588 | 1.505398 | 1.510133 | 1.512019 | 1.513144 | 1.513335 |
| | (0.000473) | (0.000189) | (0.000068) | (0.000059) | (0.000056) | (0.000132) |
| Abbe # (25) | 53.49 | 52.47 | 52.14 | 51.79 | 52.78 | 51.51 |
| | (1.63) | (0.74) | (0.08) | (0.60) | (0.72) | (0.68) |
| WC (wt. %) | 1.96 | 1.79 | 1.44 | 1.51 | 1.40 | 1.48 |
| Hydrated | (0.08) | (0.15) | (0.02) | (0.27) | (0.18) | (0.30) |
| Tan δₘₐₓ (° C.) | 18.5 | 20.1 | 22.8 | 24.1 | 24.2 | 25.3 |
| Dry | | | | | | |
| E′ (MPa) | 5.72 | 9.63 | 18.4 | 19.7 | 32.5 | 34.9 |
| Dry | (0.04) | (0.05) | (0.6) | (0.2) | (0.8) | (0.9) |

TABLE 22
Formulations and Physical Properties

| Components | | | | | |
|---|---|---|---|---|---|
| (weight %) | Ex. 126 | Ex. 127 | Ex. 128 | Ex. 129 | Ex. 130 |
| EGDCA | 57.5 | 57.5 | 55.5 | 55.5 | 55.5 |
| BCEA | 25 | 25 | 27 | 27 | 27 |
| HBA | 10 | 10 | 10 | 10 | 10 |
| NHA | 5.35 | 4.85 | 5.35 | 4.85 | 4.35 |
| TCDA | 1.5 | 2 | 1.5 | 2 | 2.5 |
| HEVC | 0.2 | 0.2 | 0.2 | 0.2 | 0.2 |
| Omnirad 819 | 0.45 | 0.45 | 0.45 | 0.45 | 0.45 |
| Σ Components | 100 | 100 | 100 | 100 | 100 |

| Properties | Ex. 126 | Ex. 127 | Ex. 128 | Ex. 129 | Ex. 130 |
|---|---|---|---|---|---|
| RI (25) | 1.513858 | 1.514288 | 1.512646 | 1.513253 | 1.513719 |
| | (0.000112) | (0.000083) | (0.000010) | (0.000072) | (0.000109) |
| Abbe # (25) | 54.02 | 51.27 | 51.69 | 53.00 | 51.62 |
| | (1.17) | (1.11) | (0.59) | (0.60) | (0.58) |
| WC (wt. %) | 1.24 | 1.37 | 1.31 | 1.41 | 1.26 |
| Hydrated | (0.05) | (0.04) | (0.10) | (0.15) | (0.03) |
| Tan δₘₐₓ (° C.) | 26 | 27 | 25.8 | 26.6 | 27.3 |
| Dry | | | | | |
| E′ (MPa) | 68.7 | 86.8 | 55.4 | 72.4 | 105 |
| Dry | (1.5) | (1.8) | (1.5) | (1.7) | (1.7) |

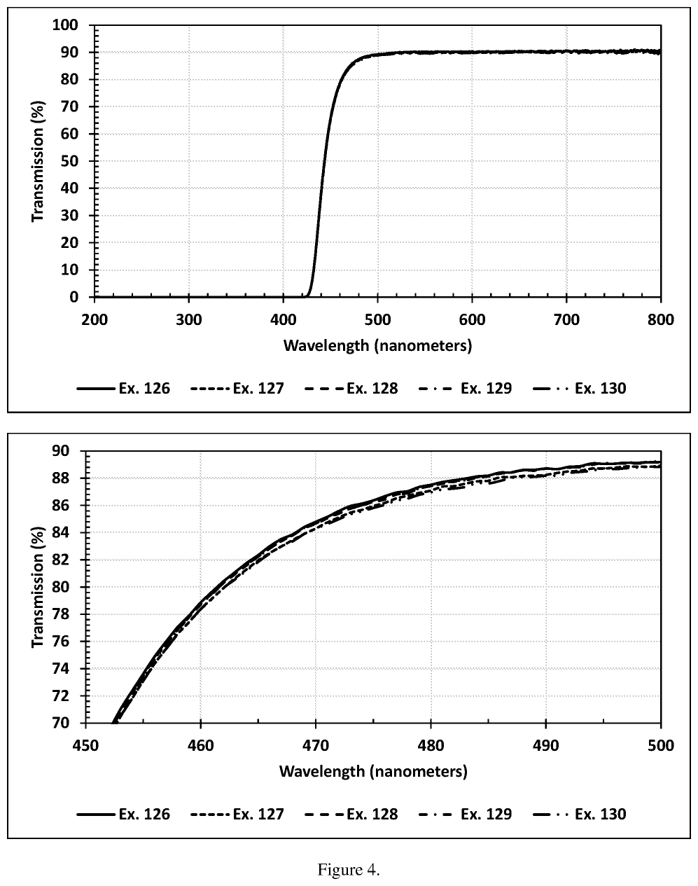

Examples 120-130 exhibited high refractive indices (greater than 1.50) and Abbe numbers (greater than 51) making these materials most suitable for ophthalmic devices such as intraocular lenses. Examples 122-130 having between 50 weight percent and 58 weight percent of EGDCA exhibited tan δmax (dry) between 22° C. and 28° C. and storage moduli (dry) between 15 MPa and 90 MPa which are desired ranges for intraocular lenses. The UV-VIS transmission spectra of Example 126-130 disks are shown in FIG. 4. These spectra were virtually identical, differing not more than 100 at any wavelength. These samples absorbed essentially all light having wavelengths between 300 nanometers and 430 nanometers and then transitioned between 430 nanometers to 500 nanometers to a level 90% transmission for longer wavelengths.

### Examples 131-135 (Prophetic)

Polymer disks are fabricated by thermally curing the RMM listed in Table 23 between 60° C. and 80° C. for 2 to 24 hours and then demolded using the standard demolding procedure (SCDEP). The amount of AIBN is varied to control the polymerization rate and reaction time. The polymer disks are extracted with acetonitrile (AAEP). Extracted disks are vacuum dried at 85° C. (<1 in Hg) to constant weight and then stored under a nitrogen gas atmosphere in a glove box at room temperature until the time of analysis. For each example, the refractive index, Abbe number, tan deltas, and storage moduli are determined on the extracted, dried disks. Some disks are subsequently hydrated for 2 weeks in deionized water at 37° C. before the physical and mechanical properties are measured to determine the impact of water content and storage conditions.

TABLE 23
Formulations

| Components | | | | | |
|---|---|---|---|---|---|
| (weight %) | Ex. 131 | Ex. 132 | Ex. 133 | Ex. 134 | Ex. 135 |
| EGDCA | 57.5 | 57.5 | 55.5 | 55.5 | 55.5 |
| BCEA | 25 | 25 | 27 | 27 | 27 |
| HBA | 10 | 10 | 10 | 10 | 10 |
| NHA | 5.35 | 4.85 | 5.35 | 4.85 | 4.35 |
| TCDA | 1.5 | 2 | 1.5 | 2 | 2.5 |
| HEVC | 0.2 | 0.2 | 0.2 | 0.2 | 0.2 |
| AIBN | 0.45 | 0.45 | 0.45 | 0.45 | 0.45 |
| Σ Components | 100 | 100 | 100 | 100 | 100 |

### Examples 136-140

Polymer disks were fabricated using the standard curing and demolding procedures (SCDEP) using the RMM listed in Table 24 and then extracted with acetonitrile (AAEP). Extracted disks were vacuum dried at 85° C. (<1 in Hg) to constant weight and then stored under a nitrogen gas atmosphere in a glove box at room temperature until the time of analysis. For each example, the refractive index, Abbe number, tan deltas, and storage moduli were determined on the extracted, dried disks (denoted as “dry” in Table 24 and equivalent to zero percent humidity). The average refractive indexes, Abbe numbers, tan deltas, and storage moduli are listed in Table 24. Standard deviations are listed in parentheses. Some disks were subsequently hydrated for 2 weeks in deionized water at 37° C. before the physical and mechanical properties were measured (denoted as “hydrated” in Table 24). Other disks were subsequently stored under controlled relative humidity (2500 or 4500 relative humidity) at ambient temperature for 24 hours before the physical and mechanical properties were measured to determine the impact of relative humidity on physical properties (denoted as “RH125 or RH45” in Table 24).

TABLE 24
Formulations and Physical Properties

| Components | | | | | |
|---|---|---|---|---|---|
| (weight %) | Ex. 136 | Ex. 137 | Ex. 138 | Ex. 139 | Ex. 140 |
| EGDCA | 74.2803 | 71.9298 | 74.2803 | 71.5 | 60 |
| BCEA | 6 | 6.1975 | 8 | 10 | 21.55 |
| HBA | 11.7803 | 14.9282 | 11.7803 | 11.55 | 12 |
| NHA | 6.2886 | 4.9975 | 4.2886 | 5 | 4.5 |
| EGDMA | 1 | 1.3 | 1 | 1.3 | 1.3 |
| HEVC | 0.2 | 0.2 | 0.2 | 0.2 | 0.2 |
| Omnirad 819 | 0.45 | 0.45 | 0.45 | 0.45 | 0.45 |
| Σ Components | 100 | 100 | 100 | 100 | 100 |

| Properties | Ex. 136 | Ex. 137 | Ex. 138 | Ex. 139 | Ex. 140 |
|---|---|---|---|---|---|
| RI (25) | 1.521548 | 1.521054 | 1.522328 | 1.521921 | 1.515350 |
| | (0.000085) | (0.000151) | (0.000133) | (0.000113) | (0.000131) |
| Abbe # (25) | 50.53 | 52.16 | 50.38 | 53.39 | 53.95 |
| | (0.08) | (1.81) | (0.41) | (0.24) | (0.38) |
| WC (wt. %) | 1.19 | 1.72 | 1.24 | 1.34 | 1.54 |
| Hydrated | (0.05) | (0.05) | (0.01) | (0.19) | (0.07) |
| Tan δₘₐₓ (° C.) | 29.4 | 29.6 | 31.5 | 30.7 | 27 |
| Dry | | | | | |
| Tan δₘₐₓ (° C.) | — | 28.4 | — | — | — |
| RH25 | | | | | |
| Tan δₘₐₓ (° C.) | — | 26.8 | — | — | — |
| RH45 | | | | | |
| E′ (MPa) | 128 | 145 | 326 | 153 | 96.5 |
| Dry | (1.2) | (0.5) | (0.9) | (1.4) | (2.2) |
| E′ (MPa) | — | 88.3 | — | — | — |
| RH25 | | (0.6) | | | |
| E′ (MPa) | — | 50.6 | — | — | — |
| RH45 | | (0.6) | | | |

Examples 136-140 exhibited high refractive indices (greater than 1.51) and Abbe numbers (greater than 50) making these materials most suitable for ophthalmic devices such as intraocular lenses. For Example 137, samples equilibrated in relative humidity of 250% or 4500 exhibited lower tan δₘₐₓ and E′ as compared to the corresponding dry materials, suggesting that these physical properties are dependent on the water content and therefore on the post-lens fabrication conditions such as drying conditions and relative humidity. Equilibrating in deionized water before sterilization may minimize or eliminate any dependency on water content.

### Examples 141-146

Polymer disks were fabricated using the standard curing and demolding procedures (SCDEP) using the RMM listed in Table 25 and then extracted with acetonitrile (AAEP). Extracted disks were vacuum dried at 85° C. (<1 in Hg) to constant weight and then stored under a nitrogen gas atmosphere in a glove box at room temperature until the time of analysis. For each example, the refractive index, Abbe number, tan deltas, and storage moduli were determined on the extracted, dried disks (denoted as “dry” in Table 25 and equivalent to zero percent humidity). The average refractive indexes, Abbe numbers, water contents, tan deltas, and storage moduli are listed in Table 25. Standard deviations are listed in parentheses. Some disks were subsequently hydrated for 2 weeks in deionized water at 37° C. before the physical and mechanical properties were measured (denoted as “hydrated” in Table 25). Other disks were subsequently stored under controlled relative humidity (25% or 45% relative humidity) at ambient temperature for 24 hours before the physical and mechanical properties were measured to determine the impact of relative humidity on physical properties (denoted as “RH25 or RH45” in Table 25).

TABLE 25
Formulations and Physical Properties

| | Ex. 141 | Ex. 142 | Ex. 143 | Ex. 144 | Ex. 145 | Ex. 146 |
|---|---|---|---|---|---|---|
| Components | | | | | | |
| (weight %) | | | | | | |
| EGDCA | 71.42 | 71.42 | 71.42 | 71.42 | 65.5 | 65.5 |
| OPEA | 8.43 | 0 | 0 | 0 | 0 | 0 |
| MOPEA | 0 | 8.43 | 0 | 0 | 0 | 0 |
| OAOEA | 0 | 0 | 8.43 | 0 | 17.35 | 0 |
| DAOEA | 0 | 0 | 0 | 8.43 | 0 | 17.35 |
| HBA | 10 | 10 | 10 | 10 | 10 | 10 |
| NHA | 8.5 | 8.5 | 8.5 | 8.5 | 5.5 | 5.5 |
| TCDA | 1 | 1 | 1 | 1 | 1 | 1 |
| HEVC | 0.2 | 0.2 | 0.2 | 0.2 | 0.2 | 0.2 |
| Omnirad 819 | 0.45 | 0.45 | 0.45 | 0.45 | 0.45 | 0.45 |
| Σ Components | 100 | 100 | 100 | 100 | 100 | 100 |
| Properties | | | | | | |
| RI (25) | 1.519818 | 1.520473 | 1.518990 | 1.518865 | 1.516715 | 1.517197 |
| | (0.000078) | (0.000060) | (0.000119) | (0.000135) | (0.000190) | (0.000107) |
| Abbe # (25) | 50.46 | 50.15 | 50.83 | 50.98 | 52.98 | 53.23 |
| | (0.13) | (0.55) | (0.29) | (0.27) | (0.25) | (0.74) |
| WC (wt. %) | 1.28 | 1.49 | 1.23 | 1.36 | 1.49 | 1.46 |
| Hydrated | (0.14) | (0.24) | (0.08) | (0.26) | (0.05) | (0.08) |
| Tan δₘₐₓ (° C.) | 28 | 27.6 | 25.4 | 24.1 | 26.9 | 21.5 |
| Dry | | | | | | |
| Tan δₘₐₓ (° C.) | 23.6 | 25.3 | 23.9 | — | — | — |
| RH25 | | | | | | |
| Tan δₘₐₓ (° C.) | 25.2 | 23.8 | 22.6 | — | — | — |
| RH45 | | | | | | |
| E′ (MPa) | 118 | 97.4 | 48.9 | 32.9 | 74.8 | 31.1 |
| Dry | (0.2) | (0.2) | (0.1) | (0.9) | (2.3) | (0.9) |
| E′ (MPa) | 72.4 | 53.1 | 28.8 | — | — | — |
| RH25 | (1.8) | (0.6) | (0.7) | | | |
| E′ (MPa) | 24.8 | 26.5 | 17.0 | — | — | — |
| RH45 | (0.3) | (0.8) | (0.5) | | | |

Examples 141-146 exhibited high refractive indices (greater than 1.51) and Abbe numbers (greater than 50) making these materials most suitable for ophthalmic devices such as intraocular lenses. For examples 141-143, samples equilibrated in relative humidity of 25% or 45% exhibited lower tan δmax and E′ as compared to the corresponding dry materials, suggesting that these physical properties are dependent on the water content and therefore on the post-lens fabrication conditions such as drying conditions and relative humidity. Equilibrating in deionized water before sterilization may minimize or eliminate any dependency on water content.

### Examples 147-151

Polymer disks were fabricated using the standard curing and demolding procedures (SCDEP) using the RMM listed in Table 26 and then extracted with acetonitrile (AAEP). Extracted disks were vacuum dried at 85° C. (<1 in Hg) to constant weight and then stored under a nitrogen gas atmosphere in a glove box at room temperature until the time of analysis. The average refractive indexes and Abbe numbers are listed in Table 26. Standard deviations are listed in parentheses. Some disks were subsequently hydrated for 2 weeks in deionized water at 37° C. before their water contents were measured (denoted as “hydrated” in Table 26).

TABLE 26
Formulations and Physical Properties

| Components | | | | | |
|---|---|---|---|---|---|
| (weight %) | Ex. 147 | Ex. 148 | Ex. 149 | Ex. 150 | Ex. 151 |
| EGDCA | 61.35 | 61.35 | 61.85 | 62.1 | 59.85 |
| BCEA | 9 | 9 | 9 | 9 | 9 |
| DAOEA | 16 | 16 | 16 | 16 | 20 |
| HBA | 9 | 9 | 9 | 9 | 9 |
| NHA | 3 | 2.5 | 2 | 1.75 | 0 |
| TCDA | 1 | 1.5 | 1.5 | 1.5 | 1.5 |
| HEVC | 0.2 | 0.2 | 0.2 | 0.2 | 0.2 |
| Omnirad 819 | 0.45 | 0.45 | 0.45 | 0.45 | 0.45 |
| Σ Components | 100 | 100 | 100 | 100 | 100 |

| Properties | Ex. 147 | Ex. 148 | Ex. 149 | Ex. 150 | Ex. 151 |
|---|---|---|---|---|---|
| RI (25) | 1.514415 | 1.515053 | 1.515854 | 1.516210 | 1.514997 |
| | (0.000398) | (0.000038) | (0.000211) | (0.000186) | (0.000095) |
| Abbe # (25) | 51.61 | 51.13 | 51.90 | 53.11 | 50.85 |
| | (0.72) | (0.23) | (0.55) | (0.22 | (0.55) |
| WC (wt. %) | 1.33 | 1.28 | 1.19 | 1.24 | 1.56 |
| Hydrated | (0.21) | (0.22) | (0.05) | (0.07) | (0.26) |

Examples 147-151 exhibited high refractive indices (greater than 1.51) and Abbe numbers (greater than 50) making these materials most suitable for ophthalmic devices such as intraocular lenses. Examples 147-151 also exhibited low water contents (about 1.2-1.6 weight percent) which can provide dimensional stability for implantable ophthalmic devices.

### Examples 152-154

Polymer disks were fabricated using the standard curing and demolding procedures (SCDEP) using the RMM listed in Table 27 and then extracted with acetonitrile (AAEP). Extracted disks were vacuum dried at 85° C. (<1 in Hg) to constant weight and then stored under a nitrogen gas atmosphere in a glove box at room temperature until the time of analysis. For each example, the refractive index, Abbe number, tan deltas, and storage moduli were determined on the extracted, dried disks (denoted as “dry” in Table 27 and equivalent to zero percent humidity). Standard deviations are listed in parentheses. Some disks were subsequently hydrated for 2 weeks in deionized water at 37° C. before their water contents were measured (denoted as “hydrated” in Table 27).

TABLE 27
Formulations and Physical Properties

| Components | | | |
|---|---|---|---|
| (weight %) | Ex. 152 | Ex. 153 | Ex. 154 |
| EGDCA | 57.5 | 57.5 | 57.5 |
| BCEA | 25 | 25.75 | 27 |
| HBA | 10 | 10 | 10 |
| NHA | 5.35 | 4.6 | 3.35 |
| TCDA | 1.5 | 1.5 | 1.5 |
| HEVC | 0.2 | 0.2 | 0.2 |
| Omnirad 819 | 0.45 | 0.45 | 0.45 |
| Σ Components | 100 | 100 | 100 |

| Properties | Ex. 152 | Ex. 153 | Ex. 154 |
|---|---|---|---|
| RI (25) | 1.513836 | 1.514070 | 1.514193 |
| | (0.000172) | (0.000207) | (0.000078) |
| Abbe # (25) | 51.94 | 51.79 | 51.13 |
| | (0.11) | (0.22) | (0.15) |
| WC (wt. %) | 1.28 | 1.54 | 1.42 |
| Hydrated | (0.08) | (0.59) | (0.03) |
| Tan δₘₐₓ (° C.) | 26.1 | 27.0 | 28.1 |
| Dry | | | |
| E′ (MPa) | 51.9 | 65.5 | 104 |
| Dry | (1.5) | (1.8) | (2.0) |

Examples 152-154 exhibited high refractive indices (greater than 1.51) and Abbe numbers (greater than 50) making these materials most suitable for ophthalmic devices such as intraocular lenses. Examples 147-151 also exhibited low water contents (about 1.2-1.6 weight percent) which can provide dimensional stability for implantable ophthalmic devices.

### Examples 155-160 (Prophetic)

Polymer disks are fabricated by thermally curing the RMM listed in Table 28 between 60° C. and 80° C. for 2 to 24 hours and then demolded using the standard demolding procedure (SCDEP). The amount of AIBN is varied to control the polymerization rate and reaction time. The polymer disks are extracted with acetonitrile (AAEP). Extracted disks are vacuum dried at 85° C. (<1 in Hg) to constant weight and then stored under a nitrogen gas atmosphere in a glove box at room temperature until the time of analysis. For each example, the refractive index, Abbe number, tan deltas, and storage moduli are determined on the extracted, dried disks. Some disks are subsequently hydrated for 2 weeks in deionized water at 37° C. before the physical and mechanical properties were measured. Other disks are subsequently stored under controlled relative humidity (25% or 45% relative humidity) at ambient temperature for 24 hours before the physical and mechanical properties are measured to determine the impact of relative humidity on physical properties.

TABLE 28
Formulations

| Components | | | | | | |
|---|---|---|---|---|---|---|
| (weight %) | Ex. 155 | Ex. 156 | Ex. 157 | Ex. 158 | Ex. 159 | Ex. 160 |
| EGDCA | 71.42 | 71.42 | 71.42 | 65.5 | 65.5 | 55.5 |
| OPEA | 8.43 | 0 | 0 | 0 | 0 | 0 |
| MOPEA | 0 | 8.43 | 0 | 0 | 0 | 0 |
| OAOEA | 0 | 0 | 8.43 | 17.35 | 0 | 0 |
| DAOEA | 0 | 0 | 0 | 0 | 17.35 | 0 |
| BCEA | 0 | 0 | 0 | 0 | 0 | 27 |
| HBA | 10 | 10 | 10 | 10 | 10 | 10 |
| NHA | 8.5 | 8.5 | 8.5 | 5.5 | 5.5 | 5.35 |
| TCDA | 1 | 1 | 1 | 1 | 1 | 1.5 |
| HEVC | 0.2 | 0.2 | 0.2 | 0.2 | 0.2 | 0.2 |
| AIBN | 0.45 | 0.45 | 0.45 | 0.45 | 0.45 | 0.45 |
| Σ Components | 100 | 100 | 100 | 100 | 100 | 100 |

### Examples 161-162 (Silicone Hydrogel Contact Lenses)

Reactive monomer mixtures were formed by mixing the reactive components listed in Table 29 with the diluent D3O such that the weight percent of the reactive components was 68.65 weight percent and the weight percent of D3O was 31.35 weight percent. These formulations were filtered through a 3 pm filter using a stainless-steel syringe and degassed by applying vacuum (about 40 mm Hg). In a glove box with a nitrogen gas atmosphere and less than about 0.2 percent oxygen gas, about 75 μL of the reactive mixture were dosed using an Eppendorf pipet at room temperature into the FC made of 90:10 (w/w) Zeonor/TT blend. The BC made of 90:10 (w/w) Z:TT blend was then placed onto the FC. The target spherical power of the mold design was nominally minus one diopter. The molds were equilibrated for a minimum of twelve hours in the glove box prior to dosing. Pallets, each containing eight mold assemblies, were transferred into an adjacent glove box maintained at about 65° C., and the lenses were cured from the top and the bottom using 435 nm LED lights having an intensity of about 2 mW/cm² at the tray's location for total of 10 minutes. The lenses were manually demolded with most lenses adhering to the FC and released by suspending the lenses in about one liter of 70 percent IPA for about one hour and then extracted one time with fresh 70% (v/v) aqueous IPA for thirty minutes, hydrated with deionized water for sixty minutes followed by two deionized water change outs for thirty minutes, and then equilibrated with packing solution two times for thirty minutes. The lenses were stored in vials in packing solution. A person of ordinary skill recognizes that the exact lens release process can be varied depending on the lens formulation and mold materials, regarding the concentrations of the aqueous isopropanol solutions, the number of washings with each solvent, and the duration of each step. The purpose of the lens release process is to release all of the lenses without defects and transition from diluent swollen networks to the packaging solution swollen hydrogels. For each example, the physical and mechanical properties of the lenses were measured and reported in Table 29.

TABLE 29
Reactive Monomer Mixtures and Physical Properties

| | Ex. 161 | Ex. 162 |
|---|---|---|
| Component | Weight Percent | Weight Percent |
| mPDMS | 30.00 | 30.00 |
| SiMAA | 28.00 | 28.00 |
| MOPEA | 0.00 | 12.00 |
| OPEA | 12.00 | 0.00 |
| HEMA | 6.65 | 6.65 |
| PVP K90 | 7.00 | 7.00 |
| TEGDMA | 2.00 | 2.00 |
| Norbloc | 2.00 | 2.00 |
| RB247 | 0.01 | 0.01 |
| Omnirad 1870 | 0.34 | 0.34 |
| Σ Components | 100 | 100 |
| Diluent | D3O | D3O |

| Properties | Ex. 161 | Ex. 162 |
|---|---|---|
| WC (wt. %) | 23 | 35 |
| Haze (%) | 4.1 ± 0.4 | 10.4 ± 0.4 |
| EC Dk (barrers) | 94 | 104 |
| Sessile Drop (degrees) | FC 55 ± 10 | FC 46 ± 9 |
| (FC/BC) | BC 56 ± 2 | BC 59 ± 4 |
| Tensile Strength (psi) | 141 ± 32 | 96 ± 16 |
| Toughness (in-lbs/in³) | 194 ± 66 | 268 ± 30 |
| Elongation (%) | 257 ± 50 | 126 ± 27 |
| Modulus (psi) | 151 ± 11 | 83 ± 6 |

The silicone hydrogel contact lenses of Examples 161 and 162 exhibited a good balance of physical and mechanical properties, making them suitable for contact lens wear.

### Examples 163-164 (Hydrogel Contact Lens)

Reactive monomer mixtures were formed by mixing the reactive components listed in Table 30 with the diluent BAGE such that the weight percent of the reactive components was 68.65 weight percent and the weight percent of D3O was 31.35 weight percent. These formulations were filtered through a 3 μm filter using a stainless-steel syringe and degassed by applying vacuum (about 40 mm Hg). In a glove box with a nitrogen gas atmosphere and less than about 0.2 percent oxygen gas, about 75 μL of the reactive mixture were dosed using an Eppendorf pipet at room temperature into the FC made of 90:10 (w/w) Zeonor/TT blend. The BC made of 90:10 (w/w) Z:TT blend was then placed onto the FC. The target spherical power of the mold design was nominally minus one diopter. The molds were equilibrated for a minimum of twelve hours in the glove box prior to dosing. Pallets, each containing eight mold assemblies, were transferred into an adjacent glove box maintained at about 65° C., and the lenses were cured from the top and the bottom using 435 nm LED lights having an intensity of about 2 mW/cm² at the tray's location for total of 10 minutes. The resulting lenses were manually demolded and then soaked in 70% (v/v) aqueous IPA (about one hour), extracted one time with 70% (v/v) aqueous IPA for thirty minutes, hydrated with deionized water for sixty minutes, but the lenses turned hazy. As a result, the lenses were soaked overnight in 75% aqueous IPA followed by extractions with 50% (v/v) aqueous IPA for sixty minutes, 30% aqueous IPA for sixty minutes, 15% aqueous IPA for sixty minutes and then by four extractions with deionized water for thirty minutes each time. The lenses were stored in vials in packing solution. A person of ordinary skill recognizes that the exact lens release process can be varied depending on the lens formulation and mold materials, regarding the concentrations of the aqueous isopropanol solutions, the number of washings with each solvent, and the duration of each step. The purpose of the lens release process is to release all of the lenses without defects and transition from diluent swollen networks to the packaging solution swollen hydrogels. For each example, the physical and mechanical properties of the lenses were measured and reported in Table 30.

TABLE 30
Reactive Monomer Mixtures and Physical Properties

| | Ex. 163 | Ex. 164 |
|---|---|---|
| Component | Weight Percent | Weight Percent |
| OPEA | 20.95 | 20.45 |
| HEMA | 77.00 | 77.00 |
| MAA | 0.00 | 0.50 |
| EGDMA | 0.70 | 0.70 |
| Norbloc | 1.00 | 1.00 |
| RB247 | 0.01 | 0.01 |
| Omnirad 1870 | 0.34 | 0.34 |
| Σ Components | 100 | 100 |
| Diluent | BAGE | BAGE |

| Properties | Ex. 163 | Ex. 164 |
|---|---|---|
| WC (wt. %) | 26 | 30 |
| Haze (%) | 4.5 ± 0.8 | 6.9 ± 1.2 |
| Sessile Drop (degrees) | FC 76 ± 1 | FC 86 ± 3 |
| (FC/BC) | BC 83 ± 1 | BC 82 ± 5 |
| Tensile Strength (psi) | 98 ± 44 | 78 ± 30 |
| Toughness (in-lbs/in³) | 170 ± 121 | 239 ± 77 |
| Elongation (%) | 292 ± 106 | 110 ± 71 |
| Modulus (psi) | 86 ± 9 | 79 ± 11 |

The hydrogel contact lenses of Examples 163 and 164 exhibited a good balance of physical and mechanical properties, making them suitable for contact lens wear.

### Examples 165-168 (Silicone Hydrogel Contact Lenses)

Reactive monomer mixtures were prepared composed of 80 weight percent of the formulations listed in Table 31 and 20 weight percent of the diluent D3O. The reactive monomer mixtures were individually filtered through a 3 μm filter using a stainless-steel syringe under pressure.

The formulations were degassed at ambient temperature by applying vacuum (40 torr) for 20 minutes. Then, in a glove box with a nitrogen gas atmosphere and less than about 0.1-0.2 percent oxygen gas, about 75 μL of the reactive mixture were dosed using an Eppendorf pipet at room temperature into the FC made of 90:10 (w/w) Zeonor/TT blend. The BC made of 90:10 (w/w) Z:PP blend was then placed onto the FC. The molds were equilibrated for a minimum of twelve hours in the glove box prior to dosing. Pallets containing eight mold assemblies each were transferred into an adjacent glove box maintained at 60° C., and the lenses were photocured (Ex. 165 and Ex. 167) from the top and the bottom using 435 nm LED lights having an intensity of about 1.6 mW/cm² at the tray's location for two minutes and then having an intensity of about 3.3 mW/cm² at the tray's location for 8 minutes, or thermally cured (Ex. 166 and Ex. 168) at 60° C. for 15-17 hours in an oven with a nitrogen gas atmosphere and less than about 0.1-0.2 percent oxygen gas.

The lenses were manually demolded with most lenses adhering to the FC and released by suspending the lenses in about one liter of 70 percent IPA for about one hour, followed by soaking with fresh 50 percent IPA for 30 minutes; by soaking with fresh 25 percent IPA for 30 minutes; then by soaking two times with fresh DIW for 30 minutes; and finally by soaking in two times in packing solution for 30 minutes. The lenses were equilibrated and stored in borate buffered packaging solution. A person of ordinary skill recognizes that the exact lens release process can be varied depending on the lens formulation and mold materials, regarding the concentrations of the aqueous isopropanol solutions, the number of washings with each solvent, and the duration of each step. The purpose of the lens release process is to release all of the lenses without defects and transition from diluent swollen networks to the packaging solution swollen hydrogels. For each example, the physical and mechanical properties of the lenses were measured and reported in Table 31.

TABLE 31
Reactive Monomer Mixtures and Physical Properties

| | Ex. 165 | Ex. 166 | Ex. 167 | Ex. 168 |
|---|---|---|---|---|
| | Weight | Weight | Weight | Weight |
| Component | Percent | Percent | Percent | Percent |
| OH-mPDMS (n = 14) | 30 | 23.92 | 30 | 23.92 |
| tBu-SiMAA | 25 | 19.92 | 25 | 19.93 |
| BHPMA-PDMS | 4 | 3.2 | 4 | 3.19 |
| DMAOEMA | 22 | 17.54 | 22 | 17.54 |
| HEMA | 7.64 | 6.1 | 0 | 0 |
| GMMA | 0 | 0 | 7.64 | 6.1 |
| RB247 | 0.01 | 0.01 | 0.01 | 0.01 |
| PVP K90 | 9 | 7.18 | 9 | 7.18 |
| EGDMA | 0.25 | 0.2 | 0.25 | 0.2 |
| Norbloc | 2 | 1.6 | 2 | 1.59 |
| Omnirad 1870 | 0.1 | 0 | 0.1 | 0 |
| AIBN | 0 | 0.4 | 0 | 0.4 |
| Σ Components | 100 | 100 | 100 | 100 |

| Properties | Ex. 165 | | Ex. 166 | | Ex. 167 | | Ex. 168 | |
|---|---|---|---|---|---|---|---|---|
| WC (wt. %) | 31 | | 28 | | 32 | | 30 | |
| Haze (%) | 63.4 | (6.3) | 32.6 | (2) | 31.3 | (4.6) | 74.3 | (5.5) |
| EC Dk (barrers) | 109 | | 80 | | 92 | | 95 | |
| Sessile Drop (degrees) | FC 98 (7) | | FC 33 (1) | | FC 102 (4) | | FC 33 (2) | |
| (FC/BC) | BC 94 (13) | | BC 34 (3) | | BC 99 (3) | | BC 35 (4) | |
| Cahn DCA (Adv./Rec) | Adv 33 (26) | | Adv 41 (6) | | Adv 69 (12) | | Adv 51 (9) | |
| | Rec 35 (7) | | Rec 34 (8) | | Rec 43 (12) | | Rec 53 (5) | |
| Elongation (%) | 260 | (37) | 246 | (49) | 222 | (43) | 174 | (31) |
| Modulus (psi) | 408 | (47) | 479 | (39) | 298 | (37) | 390 | (26) |

The silicone hydrogel contact lenses of Examples 165-168 exhibited a good balance of physical and mechanical properties. A skilled person could optimize the formulation to reduce the haze values and moduli, for instance, by adjusting the concentrations of DMAOEMA, THEMA, and EGDMA and/or by using another cross-linking agent and/or by changing the diluent and/or its concentration.

### Example 169

A reactive monomer mixture was prepared composed of 80 weight percent of the formulation listed in Table 32 and 20 weight percent of the diluent D3O. The reactive monomer mixture was filtered through a 3 m filter using a stainless-steel syringe under pressure.

The resulting formulation was degassed at ambient temperature by applying vacuum (40 torr) for 20 minutes. Then, in a glove box with a nitrogen gas atmosphere and less than about 0.1-0.2 percent oxygen gas, about 75 μL of the reactive mixture were dosed using an Eppendorf pipet at room temperature into the FC made of Zeonor. The BC made of 90:10 (w/w) Z:PP blend was then placed onto the FC. The molds were equilibrated for a minimum of twelve hours in the glove box prior to dosing. Pallets each containing eight mold assemblies were transferred into a convection oven set at 75° C. in an adjacent glove box having a nitrogen gas atmosphere and less than about 0.1-0.2 percent oxygen gas, and the lenses were thermally cured for 60 minutes.

The lenses were manually demolded with most lenses adhering to the FC and released by suspending the lenses in about one liter of 70 percent IPA for about one hour, followed by soaking with fresh 50 percent IPA for 30 minutes; by soaking with fresh 25 percent IPA for 30 minutes; then by soaking two times with fresh DIW for 30 minutes; and finally by soaking in two times in packing solution for 30 minutes. The lenses were equilibrated and stored in borate buffered packaging solution. A person of ordinary skill recognizes that the exact lens release process can be varied depending on the lens formulation and mold materials, regarding the concentrations of the aqueous isopropanol solutions, the number of washings with each solvent, and the duration of each step. The purpose of the lens release process is to release all of the lenses without defects and transition from diluent swollen networks to the packaging solution swollen hydrogels. For each example, the physical and mechanical properties of the lenses were measured and reported in Table 32.

TABLE 32
Reactive Monomer Mixtures and Physical Properties

| | Ex. 169 |
|---|---|
| Component | Weight Percent |
| OH-mPDMS (n = 14) | 20 |
| OH-mPDMS (n = 4) | 20 |
| tBu-SiMAA | 19 |
| DMAOEMA | 22 |
| HEMA | 7.1 |
| RB247 | 0.01 |
| PVP K90 | 9 |
| EGDMA | 0.75 |
| Norbloc | 1.98 |
| AIBN | 0.16 |
| Σ Components | 100 |

| Properties | Ex. 169 | |
|---|---|---|
| WC (wt. %) | 30 | |
| Haze (%) | 40 | (0.8) |
| EC Dk (barrers) | 93 | |
| Sessile Drop (degrees) | FC 36 (6) | |
| (FC/BC) | BC 35 (1) | |
| Cahn DCA (Adv./Rec) | Adv 49 (2) | |
| | Rec 47 (2) | |
| Elongation (%) | 490 | (121) |
| Modulus (psi) | 368 | (36) |

The silicone hydrogel contact lenses of Example 169 exhibited a good balance of physical and mechanical properties. A skilled person could optimize the formulation to reduce the haze values and moduli, for instance, by adjusting the concentrations of DMAOEMA, HEMA, and EGDMA and/or by using another cross-linking agent and/or by changing the diluent and/or its concentration.

### Example 170 (Hompolymeric Network)

Polymer disks were fabricated using the standard curing, demolding, and extraction procedures (SCDEP) using a reactive monomer mixture composed of 98 weight percent DMAOEMA, 1.9 weight percent EGDMA, and 0.1 weight percent Omnirad 1870 with the following exceptions to the curing, extraction, and hydration conditions: (1) disks were photocured at 60° C. from the top and the bottom using 435 nm LED lights having an intensity of about 1.6 mW/cm² at the tray's location for two minutes and then having an intensity of about 3.3 mW/cm² at the tray's location for 8 minutes: (2) disks were extracted with DIW (25 mL per disk) three times for thirty minutes; and (3) disks were equilibrated in packing solution. The water content was determined using the contact lens test method and was found to be 58.5 weight percent. The polymeric networked swelled about 27 percent based on the difference between wet and dry lens diameters.

### Examples 171 (Prophetic Homopolymers)

18.4 grams (92.1 mmol) OPEA, 24.8 grams (92.1 mmol) DAOEA, or 20.0 grams (92.1 mmol) DHOEMA and 40 milligrams (0.2 mmol) AIBN are added into a 1-liter reactor and dissolved in about 250 mL of 50:50 (v/v) aqueous methanol. The solution is degassed by bubbling nitrogen gas through the system for about 15 minutes at room temperature. The reaction mixture is heated at 60-62° C. under a nitrogen gas atmosphere for about 12 hours and then cooled to room temperature. The solvent is evaporated under reduced pressure. Acetone is added to the residue. The resulting mixture is heated to 62° C. for 12 hours with constant stirring; thereafter, the mixture is cooled to room temperature. Upon standing for two hours at room temperature, the insoluble solids settle. The acetone is decanted off and discarded. The crude product is rinsed for two additional hours in acetone at room temperature with stirring. The acetone is decanted off and discarded. The homopolymer is then vacuum dried at 60-65° C. to constant weight and characterized by NMR spectroscopy (chemical composition) and gel permeation or size exclusion chromatography (molecular weight and molecular weight distribution).

### Example 172 (Prophetic Copolymer)

20.0 grams (92.1 mmol) DHOEMA, 18.4 grams (92.1 mmol) OPEA, and 40 milligrams (0.2 mmol) AIBN are added into a 1-liter reactor and dissolved in about 250 mL of 50:50 (v/v) aqueous methanol and another more suitable aqueous-organic solvent combination. The molar ratio of monomers may vary. The solution is degassed by bubbling nitrogen gas through the system for about 15 minutes at room temperature. The reaction mixture is heated at 60-62° C. under a nitrogen gas atmosphere for about 12 hours and then cooled to room temperature. The solvent is evaporated under reduced pressure. Acetone is added to the residue. The resulting mixture is heated to 62° C. for 12 hours with constant stirring; thereafter, the mixture is cooled to room temperature. Upon standing for two hours at room temperature, the insoluble solids settle. The acetone is decanted off and discarded. The crude product is rinsed for two additional hours in acetone at room temperature with stirring. The acetone is decanted off and discarded. The copolymer is then vacuum dried at 60-65° C. to constant weight and characterized by NMR spectroscopy (chemical composition) and gel permeation size exclusion chromatography (molecular weight and molecular weight distribution).

1. A composition made by free radical polymerization of a reactive monomer mixture comprising:
a) a compatibilizing monomer selected from the group consisting of a pendant carbamate monomer having a chemical structure of Formula I, a pendant amide monomer having a chemical structure of Formula II, and combinations thereof,
P_{g}-L-OCONR¹R² Formula I
P_{g}-L-CONR¹R² Formula II,
wherein P_{g} is a polymerizable group, L is a linking group, R¹ and R² are independently selected from H, alkyl, haloalkyl, alkoxyalkyl, hydroxyalkyl, amidoalkyl, cycloalkyl, cycloalkyl(alkyl), heterocycloalkyl, aryl, arylalkyl, heteroaryl, and heteroarylalkyl groups;
b) a cross-linking agent; and
c) an ethylene glycol dicyclopentenyl ether (meth)acrylate;
wherein the concentration of the ethylene glycol dicyclopentenyl ether (meth)acrylate in the reactive monomer mixture excluding any diluent is greater than or equal to 20 weight percent; and wherein the composition exhibits a refractive index of at least 1.45 and an Abbe number of at least 39.

2. The composition of claim 1, wherein the polymerizable group of the compatibilizing monomer is a (meth)acrylate and the linking group of the compatibilizing monomer is an unsubstituted alkylene group.

3. The composition of claim 2, wherein the compatibilizing monomer is selected from the group consisting of Formula III, Formula IV, and combinations thereof: 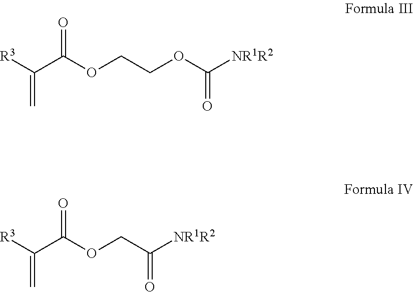
wherein R³ is H or methyl.

4. The composition of claim 3, wherein the compatibilizing monomer is selected from the group consisting of: 2-((methylcarbamoyl)oxy)ethyl (meth)acrylate, 2-((ethylcarbamoyl)oxy)ethyl (meth)acrylate, 2-((propylcarbamoyl)oxy)ethyl (meth)acrylate, 2-((butylcarbamoyl)oxy)ethyl (meth)acrylate, 2-((pentylcarbamoyl)oxy)ethyl (meth)acrylate,
2-((hexylcarbamoyl)oxy)ethyl (meth)acrylate, 2-((heptylcarbamoyl)oxy)ethyl (meth)acrylate, 2-((octylcarbamoyl)oxy)ethyl (meth)acrylate, 2-((nonylcarbamoyl)oxy)ethyl (meth)acrylate, 2-((decylcarbamoyl)oxy)ethyl (meth)acrylate, 2-((undecylcarbamoyl)oxy)ethyl (meth)acrylate, 2-((dodecylcarbamoyl)oxy)ethyl (meth)acrylate, 2-((tridecylcarbamoyl)oxy)ethyl (meth)acrylate, 2-((tetradecylcarbamoyl)oxy)ethyl (meth)acrylate, 2-((pentadecylcarbamoyl)oxy)ethyl (meth)acrylate, 2-((hexadecylcarbamoyl)oxy)ethyl (meth)acrylate, 2-((heptadecylcarbamoyl)oxy)ethyl (meth)acrylate, 2-oxo-2-(methylamino)ethyl (meth)acrylate, 2-oxo-2-(ethylamino)ethyl (meth)acrylate, 2-oxo-2-(propylamino)ethyl (meth)acrylate, 2-oxo-2-(butylamino)ethyl (meth)acrylate, 2-oxo-2-(pentylamino)ethyl (meth)acrylate, 2-oxo-2-(hexylamino)ethyl (meth)acrylate, 2-oxo-2-(heptylamino)ethyl (meth)acrylate, 2-oxo-2-(octylamino)ethyl (meth)acrylate, 2-oxo-2-(nonylamino)ethyl (meth)acrylate, 2-oxo-2-(decylamino)ethyl (meth)acrylate, 2-oxo-2-(undecylamino)ethyl (meth)acrylate, 2-oxo-2-(dodecylamino)ethyl (meth)acrylate, 2-oxo-2-(tridecylamino)ethyl (meth)acrylate, 2-oxo-2-(tetradecylamino)ethyl (meth)acrylate, 2-oxo-2-((3-methoxypropyl)amino)ethyl (meth)acrylate, 2-oxo-2-((3-ethoxypropyl)amino)ethyl (meth)acrylate, 2-oxo-2-((cyclohexylmethyl)amino)ethyl (meth)acrylate, 2-oxo-2-(benzylamino)ethyl (meth)acrylate, 2-oxo-2-(phenethylamino)ethyl (meth)acrylate, 2-oxo-2-((thiophen-2-ylmethyl)amino)ethyl (meth)acrylate, 2-oxo-2-((2,3-dihydroxypropyl)amino)ethyl (meth)acrylate, 2-(dimethylamino)-2-oxoethyl methacrylate, and combinations thereof.

5. (canceled)

6. (canceled)

7. The composition of claim 1, wherein the crosslinking agent is selected from the group consisting of tricyclo[5.2.1.0^{2,6}]decanedimethanol di(meth)acrylate, ethylene glycol di(meth)acrylate, diethylene glycol di(meth)acrylate, triethylene glycol di(meth)acrylate, tetraethylene glycol di(meth)acrylate, trimethylolpropane tri(meth)acrylate, 1,4-butanediol di(meth)acrylate, 1,5-pentanediol di(meth)acrylate, 1,6-hexanediol di(meth)acrylate, 1,7-heptanediol di(meth)acrylate, 1,8-octanediol di(meth)acrylate, 1,9-nonanediol di(meth)acrylate, 1,10-decanediol di(meth)acrylate, 1,11-undecanediol di(meth)acrylate, 1,12-dodecanediol di(meth)acrylate, 1,13-tridecanediol di(meth)acrylate, 1,14-tetradecanediol di(meth)acrylate, 1,15-pentadecanediol di(meth)acrylate, 1,16-hexadecanediol di(meth)acrylate, 1,17-heptadecanediol di(meth)acrylate, 1,18-octadecanediol di(meth)acrylate, glycerol tri(meth)acrylate, triallyl cyanurate, methylene bis(meth)acrylamide, poly(ethylene glycol) di(meth)acrylate, and any combination thereof.

8. (canceled)

9. (canceled)

10. The composition of claim 1, wherein the ethylene glycol dicyclopentenyl ether (meth)acrylate is ethylene glycol dicyclopentenyl ether acrylate.

11. The composition of claim 1, further comprising an aliphatic alkyl (meth)acrylate monomer, wherein the alkyl group contains between one and twenty carbon atoms.

12. (canceled)

13. The composition of claim 12, wherein the reactive monomer mixture comprises n-hexyl acrylate in an amount between about 0.01 and about 20 weight percent, between about 1 weight percent and 20 weight percent, between about 1 weight percent and about 15 weight percent, or between about 1 weight percent and about 10 weight percent.

14. The composition of claim 1, further comprising a hydroxyalkyl (meth)acrylate monomer, wherein the hydroxyalkyl group contains between one and twenty carbon atoms.

15. (canceled)

16. (canceled)

17. (canceled)

18. The composition of claim 17, wherein the free radical polymerization initiator is a photo-initiator.

19. (canceled)

20. (canceled)

21. The composition of claim 17, wherein the reactive monomer mixture comprises the free radical polymerization initiator in an amount between about 0.01 weight percent and about 5 weight percent, between about 0.1 weight percent and about 3 weight percent, between about 0.1 weight percent and about 2 weight percent, between about 0.1 weight percent and about 1 weight percent, or between about 0.2 weight percent and about 0.6 weight percent.

22. The composition of claim 1, wherein the reactive monomer mixture further comprises at least one UV absorbing compound.

23. (canceled)

24. The composition of claim 1, wherein the refractive monomer mixture further comprises at least one UV/HEV absorbing compound selected from the group consisting of 2-(2′-hydroxy-5-methacrylyloxyethylphenyl)-2H-benzotriazole, 2-(2-cyano-2-(9H-thioxanthen-9-ylidene)acetamido)ethyl methacrylate, 3-((9-(dicyanomethylene)-9H-xanthen-3-yl)oxy)propyl methacrylate, 2-(2-cyano-2-(9H-xanthen-9-ylidene)acetamido)ethyl methacrylate, 2-(2-cyano-2-(10-methylacridin-9(10H)-ylidene)acetamido)ethyl methacrylate, 3-(3-(tert-butyl)-5-(5-chloro-2H-benzo[d][1,2,3]triazol-2-yl)-4-hydroxyphenyl)propyl methacrylate, or any combination thereof.

25. (canceled)

26. (canceled)

27. The composition of claim 1, further comprising a hydrophilic component selected from the group consisting of poly(ethylene glycol) (meth)acrylate, poly(ethylene glycol) methyl ether (meth)acrylate, poly(ethylene glycol) phenyl ether (meth)acrylate, and combinations thereof.

28. (canceled)

29. The composition of claim 1, wherein the reactive monomer mixture comprises the compatibilizing monomer in an amount between about 0.01 weight percent and about 55 weight percent, between about 1 weight percent and about 40 weight percent, between about 5 weight percent and about 35 weight percent, between about 10 weight percent and about 30 weight percent, or between about 20 weight percent and about 30 weight percent.

30. The composition of claim 1, wherein the reactive monomer mixture comprises the cross-linking agent in an amount between about 0.1 weight percent and about 10 weight percent; between about 0.1 weight percent and about 5 weight percent; between about 0.5 weight percent and about 3 weight percent; or between about 1 weight percent and 3 weight percent.

31. The composition of claim 1, wherein the reactive monomer mixture comprises the ethylene glycol dicyclopentenyl ether (meth)acrylate in an amount between about 25 weight percent and about 95 weight percent, between about 30 weight percent and about 75 weight percent, between about 40 weight percent and about 65 weight percent, or between about 45 weight percent and about 60 weight percent.

32. The composition of claim 1, wherein the reactive monomer mixture further comprises at least one diluent.

33. The composition of claim 1, wherein the composition has a refractive index of at least 1.45 and an Abbe number of at least 45; wherein the composition has a refractive index of at least 1.48 and an Abbe number of at least 48; wherein the composition has a refractive index of at least 1.49 and an Abbe number of at least 49; wherein the composition has a refractive index of at least 1.50 and an Abbe number of at least 50; wherein the composition has a refractive index of at least 1.51 and an Abbe number of at least 51; or wherein the composition has a refractive index of at least 1.52 and an Abbe number of at least 52.

34. The composition of claim 1, wherein the composition exhibits a water content between about 0.01 weight percent and about 15 weight percent; between about 0.1 weight percent and about 10 weight percent; between about 0.5 weight percent and about 5 weight percent; between about 0.5 weight percent and about 3 weight percent; or between about 1 weight percent and about 2 weight percent.

35. The composition of claim 1, wherein the composition exhibits a storage modulus between about 1 megapascal and about 100 megapascals; between about 10 megapascal and about 90 megapascals; between about 20 megapascal and about 80 megapascals; between about 30 megapascal and about 80 megapascals; or between about 40 megapascal and about 80 megapascals.

36. An ophthalmic device comprising the composition of claim 1.

37. The ophthalmic device of claim 36 wherein the ophthalmic device comprises an intraocular lens, phakic intraocular lens, contact lens, corneal inlay, corneal outlay, or corneal insert.

38. (canceled)

39. The ophthalmic device of claim 36 wherein the intraocular lens is coated.

40. A method for making an ophthalmic device, the method comprising:
a. providing a composition made by free radical polymerization of a reactive monomer mixture of claim 1; and
b. forming an ophthalmic device.

41. A method for making an ophthalmic device, the method comprising:
a. preparing a blank from the composition made by free radical polymerization of a reactive monomer mixture of claim 1; and
b. machining an ophthalmic device from the blank.

42. A method for making an ophthalmic device, the method comprising:
a. molding the device from the composition made by free radical polymerization of a reactive monomer mixture of claim 1.

43-53. (canceled)

54. A composition made by free radical polymerization of a reactive monomer mixture comprising:
a) 2-((butylcarbamoyl)oxy)ethyl acrylate at 24-28 weight percent;
b) 4-hydroxybutyl acrylate at 10 weight percent;
c) tricyclo[5.2.1.0^{2,6}]decanedimethanol diacrylate at 1.5 weight percent;
d) 3-((9-(dicyanomethylene)-9H-xanthen-3-yl)oxy)propyl methacrylate at 0.2 weight percent;
e) bis(2,4,6-trimethylbenzoyl)-phenylphosphine oxide at 0.45 weight percent;
f) ethylene glycol dicyclopentenyl ether acrylate at 55-59 weight percent; and
g) n-hexyl acrylate at 3-6 weight percent;
wherein the concentration of ethylene glycol dicyclopentenyl ether acrylate and n-hexyl acrylate vary, but the components of the reactive monomer mixture add up to 100 weight percent; wherein the composition exhibits a refractive index of at least 1.50 and an Abbe number of at least 50; and wherein the storage modulus is between 1 megapascal and 100 megapascals.

55. A composition made by free radical polymerization of a reactive monomer mixture comprising:
a) 2-oxo-2-(decylamino)ethyl (meth)acrylate at 16-18 weight percent;
b) 4-hydroxybutyl acrylate at 10 weight percent;
c) tricyclo[5.2.1.0^{2,6}]decanedimethanol diacrylate at 1.5 weight percent;
d) 3-((9-(dicyanomethylene)-9H-xanthen-3-yl)oxy)propyl methacrylate at 0.2 weight percent;
e) bis(2,4,6-trimethylbenzoyl)-phenylphosphine oxide at 0.45 weight percent;
f) ethylene glycol dicyclopentenyl ether acrylate at 65-67 weight percent; and
g) n-hexyl acrylate at 5-7 weight percent;
wherein the concentration of 2-oxo-2-(decylamino)ethyl (meth)acrylate, ethylene glycol dicyclopentenyl ether acrylate, and n-hexyl acrylate vary, but the components of the reactive monomer mixture add up to 100 weight percent; wherein the composition exhibits a refractive index of at least 1.50 and an Abbe number of at least 50; and wherein the storage modulus is between 1 megapascal and 100 megapascals.

56. An ophthalmic device made from the compositions of claim 54.

57-68. (canceled)

69. An ophthalmic device made from the compositions of claim 55.
